SEQUENCE LISTING (260 sequences)

SEQ ID NO: 1 - protein, 300 aa, Saccharomyces cerevisiae
MVNNSQHSYIKDGWFREINDKSFPGQAFTMTVDSILYEARSEFQDILIFRNKVYGTVLVLDGIVQCTEFDEFAYQEMITHIAMFAHSNPKRVLIIGGGDGGVLREVAKHSCVEDITMVEIDSSVIELSRKFLPTLSNGAFDDERLDLKLCDGFKFLQDIGASDVHKKFDVIITDSSDPEGPAEAFFQERYFELLKDALNPNGVVIMQSSENFWLNLKYLHDLKNTAKKVFPNTEYCYTMVPTYTSGQLGLIVCSNNANIPLNIPQRKISEQEQGKLKYYNPQIHSSAFVLPTWADKVINE

SEQ ID NO: 2 - DNA, 904 bp, Saccharomyces cerevisiae
atggttaacaactctcaacatccatacatcaaggatggttggttcagagaaattaatgataagtcattcccaggtcaagcttttactatgacagttgattctatcttgtacgaagcaagatcagaatttcaagatatcttgatttttagaaataaggtttacggtactgttttggttttggatggtatcgttcaatgtacagaatttgatgaatttgcttaccaagaaatgatcactcatatcgctatgttcgcacattctaacccaaaaagagttttgatcattggtggtggtgacggtggtgttttgagagaagttgcaaagcattcatgtgttgaagatatcactatggttgaaatcgattcttcagttattgaattgtctagaaagttcttaccaacattgtcaaatggtgctttcgatgatgaaagattggatttgaagttgtgtgatggttttaaattcttgcaagatatcggtgcatctgatgttcataagaaattcgatgttatcatcactgattcttcagatccagaaggtccagctgaagctttctttcaagaaagatacttcgaattgttgaaggatgctttgaacccaaacggtgttgttattatgcaatcttcagaaaacttctggttgaatttgaagtatttgcatgatttgaaaaatacagctaagaaagttttcccaaacactgaatactgttacacaatggttccaacttacacatctggtcaattaggtttgatcgtttgttcaaacaacgctaacatcccattgaacatcccacaaagaaaaatttctgaacaagaacagggtaaattgaagtactacaacccacaaatccattcttcagcttttgttttgccaacatgggcagataaagttattaatgaataag

SEQ ID NO: 3 - protein, 359 aa, Arabidopsis thaliana
MEGDVGKGLVCQNTMDGKASNGNGLEKTVPSCCLKAMACVPEDDAKCHSTVVSGWFSEPHPRSGKKGGKAVYFNNPMWPGEAHSLKVEKVLFKDKSDFQEVLVFESATYGKVLVLDGIVQLTEKDECAYQEMIAHLPLCSISSPKNVLVVGGGDGGVLREISRHSSVEVIDICEIDKMVIDVSKKFFPELAVGFDDPRVQLHIGDAAEFLRKSPEGKYDAIIVDSSDPVGPALALVEKPFFETLARALKPGGVLCNMAESMWLHTHLIEDMISICRQTFKSVHYAWSSVPTYPSGVIGFVLCSTEGPAVDFKNPINPIEKLDGAMTHKRELKFYNSDMHRAAFALPTFLRREVASLLAS

SEQ ID NO: 4 - DNA, 1080 bp, Arabidopsis thaliana
atggaaggtgacgttggtaaaggtttggtttgtcaaaatactatggatggtaaagcttcaaatggtaatggtttggaaaagactgttccatcttgttgtttaaaagctatggcatgtgttccagaagatgatgcaaaatgtcattctactgttgtttcaggttggttttctgaaccacatccaagatcaggtaaaaagggtggtaaagctgtttacttcaacaacccaatgtggccaggtgaagcacattctttgaaggttgaaaaggttttgtttaaagacaagtcagattttcaagaagttttggttttcgaatctgctacttacggtaaagttttggttttggatggtatcgttcaattgacagaaaaggatgaatgtgcttaccaagaaatgattgcacatttgccattgtgttctatctcttcacctaaaaatgttttggttgttggtggtggtgacggtggtgttttgagagaaatctcaagacattcttcagttgaagttattgatatctgtgaaatcgataagatggttattgatgtttctaagaaatttttcccagaattagctgttggttttgatgatccaagagttcaattgcatattggtgacgctgcagaatttttgagaaagtcaccagagggtaaatacgatgcaatcatcgttgattcttcagatccagttggtccagctttggcattggttgaaaagccatttttcgaaacattggctagagcattaaaaccaggtggtgttttgtgtaatatggctgaatctatgtggttgcatactcatttgatcgaagatatgatctcaatctgtagacaaacttttaaatctgttcattacgcatggtcttcagttccaacttacccatcaggtgttattggtttcgttttgtgttctacagaaggtccagctgttgatttcaagaacccaattaatccaatcgaaaaattggatggtgcaatgactcataagagagaattgaagttctacaattctgatatgcatagagctgcatttgctttgccaacatttttaagaagagaagttgcttcattgttagcatcttaa

SEQ ID NO: 5 - protein, 339 aa, Arabidopsis thaliana
MGEAVEVMFGNGFPEIHKATSPTQTLHSNQQDCHWYEETIDDDLKWSFALNSVLHQGTSEYQDIALLDTKRFGKVLVIDGKMQSAERDEFIYHECLIHPALLFHPNPKTVFIMGGGEGSAAREILKHTTIEKVVMCDIDQEVVDFCRRFLTVNSDAFCNKKLELVIKDAKAELEKREEKFDIIVGDLADPVEGGPCYQLYTKSFYQNILKPKLSPNGIFVTQAGPAGIFTHKEVFTSIYNTMKQVFKYVKAYTAHVPSFADTWGWVMASDHEFDVEVDEMDRRIEERVNGELMYLNAPSFVSAATLNKTISLALEKETEVYSEENARFIHGHGVAYRHI

SEQ ID NO: 6 - DNA, 1020 bp, Arabidopsis thaliana
atgggtgaagcagtagaagtaatgttcggtaacggtttcccagaaatcttaaaagccacaagtccaactcaaaccttgcactccaatcaacaagattgtcattggtacgaagaaactatcgatgatgatttgaagtggtctttcgctttaaattctgttttgcatcaaggtacttctgaataccaagatatcgcattgttggatacaaagagattcggtaaagttttggttattgatggtaaaatgcaatcagctgaaagagatgagtttatatatcatgaatgtttgatccatccagcattgttgttccatccaaacccaaagactgtttttattatgggtggtggtgaaggttctgctgcaagagaaatcttgaagcatactacaatcgaaaaagttgttatgtgtgatatcgatcaagaagttgttgatttctgtagaagatttttgacagttaattcagatgctttctgtaataagaaattggaattagttattaaagatgctaaggcagaattggaaaagagagaagaaaagttcgatattattgttggtgacttggctgatccagttgaaggtggtccatgttatcaattgtacactaagtctttctaccaaaacattttgaaaccaaaattatcaccaaatggtatttttgttactcaagctggtccagcaggtatttttacacataaagaagtttttacttctatctataacacaatgaagcaagtttttaaatacgttaaagcttacactgcacatgttccatcttttgctgatacatggggttgggttatggcatcagatcatgaatttgatgttgaagttgatgaaatggatagaagaatcgaagaaagagttaacggtgaattgatgtacttaaatgctccatcttttgtttcagctgcaactttgaataagacaatctcattggcattggaaaaggaaacagaagtttactccgaagaaaatgctagattcatccacggtcacggtgttgcctacagacatatctaa

SEQ ID NO: 7 - protein, 370 aa, Senecio vernalis
MAESNKEAIDSARSNVFKESESLEGTCAKIGGYDFNNGIDHSKLLKSMVSTGFQASNLGDAMIITNQMLDWRLSHDEVPENCSEEEKKNRESVKCKIFLGFTSNLISSGVRETICYLTQHRMVDVLVTTTGGIEEDFIKCLASTYKGKFSLPGADLRSKGLNRIGNLIVPNDNYIKFEDWIIPIFDQMLIEQKTQNVLWTPSRMIARLGKEINNETSYLYWAYKNNIPVFCPSITDGSIGDMLYFHSVSNPGPGLVVDIVQDVIAMDNEAVHASPQKTGIIILGGGLPKHHICNANMMRNGADFAVFINTAQEYDGSDSGARPDEAVSWGKISSTGKAVKVHCDATIAFPLLVAETFAVKKEKASKVNGF

SEQ ID NO: 8 - DNA, 1113 bp, Senecio vernalis
atggctgaatctaataaggaagctatcgattctgcaagatcaaacgtttttaaagaatctgaatcattagaaggtacatgtgcaaagatcggtggttacgatttcaacaacggtatcgatcattcaaagttgttgaagtctatggtttcaacaggtttccaagcttctaatttgggtgacgcaatgatcatcactaaccaaatgttggattggagattatctcatgatgaagttccagaaaactgttcagaagaagaaaagaaaaatagagaatctgttaagtgtaagattttcttgggttttacatcaaatttgatctcttcaggtgttagagaaacaatctgttatttgactcaacatagaatggttgatgttttggttactacaactggtggtatcgaagaagatttcatcaagtgtttagcttctacttacaagggtaaattttcattgccaggtgcagatttgagatctaagggtttgaacagaattggtaatttgatcgttccaaacgataactacatcaagttcgaagattggattattccaatttttgatcaaatgttaattgaacaaaagactcaaaatgttttgtggactccatcaagaatgattgctagattgggtaaagaaattaataacgaaacatcttatttgtactgggcatacaagaacaacatcccagttttctgtccatctattactgatggttcaattggtgacatgttgtacttccattctgtttcaaacccaggtccaggtttggttgttgatatcgttcaagatgttatcgctatggataatgaagctgttcatgcatctccacaaaagactggtatcatcatcttgggtggtggtttaccaaagcatcatatctgtaacgctaacatgatgagaaacggtgctgatttcgcagtttttattaacacagcacaagaatacgatggttctgattcaggtgctagaccagatgaagcagtttcatggggtaaaatctcttcaactggtaaagctgttaaagttcattgtgatgctacaattgcatttccattgttagttgctgaaactttcgcagttaagaaagaaaaggcttctaaagttaatggtttttaa

SEQ ID NO: 9 - protein, 280 aa, Blastochloris viridis
MTDWPVYHRIDGPIVMIGFGSIGRGTLPLIERHFAFDRSKLVVIDPSDEARKLAEARGVRFIQQAVTRDNYRDLLVPLLTAGPGQGFCVNLSVDTSSLDIMELARENGALYIDTVVEPWLGFYFDPDLKPEARSNYALRETVLAARRNKPGGTTAVSCCGANPGMVSWFVKQALVNLAADLGVTREEPTTREEWARLAMDLGVKGIHIAERDTQRANFPKPFDVFVNTWSVEGFVSEGLQPAELGWGTFERWMPDNARGHDSGCGAGIYLLQPGANTRVR

SEQ ID NO: 10 - DNA, 1434 bp, Blastochloris viridis
atgacagattggccagtttaccatagaatcgatggtccaatcgttatgattggttttggttctattggtagaggtactttgccattgatcgaaagacatttcgcattcgatagatctaagttggttgttattgatccatcagatgaagctagaaaattggctgaagcaagaggtgttagattcattcaacaagcagttacaagagataactacagagatttgttggttccattgttaactgctggtccaggtcaaggtttctgtgttaatttgtctgttgatacatcttcattggatatcatggaattggctagagaaaatggtgcattgtatattgatactgttgttgaaccatggttgggtttctacttcgatccagatttgaagccagaagctagatcaaactacgcattgagagaaacagttttagctgcaagaagaaataagccaggtggtactacagctgtttcttgttgtggtgcaaatcctggtatggtttcatggttcgttaagcaagctttggttaatttggctgcagatttgggtgttactagagaagaaccaactacaagagaagaatgggctagattggcaatggatttgggtgttaagggtattcatatcgctgaaagagatacacaaagagcaaacttcccaaagccattcgatgttttcgttaacacttggtctgttgaaggttttgtttcagaaggtttgcaaccagctgaattaggttggggtacatttgaaagatggatgccagataatgctagaggtcatgattctggttgtggtgcaggtatatatttgttacaaccaggtgctaatactagagttagatcatggactccaacagctactgcacaatacggtttcttggttacacataacgaatctatctcaatcgcagatttcttgactgttagagatgctgcaggtcaagctgtttatagaccaacatgtcattatgcttaccatccatgtaacgatgcagttttgtctttgcatgaaatgtttggttctggtaaaagacaatcagattggttgatcttggatgaaactgaaatcgttgatggtatcgatgaattgggtgttttgttgtacggtcatggtaaaaatgcttattggtacggttctcaattgtcaatcgaagaaacaagaagaattgctccagatcaaaatgcaactggtttgcaagtttcttcagctgttttagctggtatggtttgggctttggaaaatccaaaagctggtattgttgaagcagatgatttggattacagaagatgtttggaagttcaaacaccatatttgggtccagttgttggtgtttacacagattggactccattggcaggtagaccaggtttatttccagaagatattgatgcttcagatccatggcaattcagaaatgttttggttagagattaa

SEQ ID NO: 11 - DNA, 1365 bp, Saccharomyces cerevisiae
atgtcagagccagaatttcaacaagcttacgaagaagttgtctcctctttggaagactctactcttttcgaacaacacccagaatacagaaaggttttgccaattgtttctgttccagaaagaatcatacaattcagagtcacctgggaaaatgacaagggtgaacaagaagttgctcaaggttacagagtgcaatataactccgccaagggtccatacaagggtggtctacgtttccatccttccgtgaacttgtctatcttgaaattcttgggtttcgaacaaatcttcaagaactccttgaccggcctagacatgggtggtggtaaaggtggtctatgtgtggacttgaagggaagatctaataacgaaatcagaagaatctgttatgctttcatgagagaattgagcagacacattggtcaagacactgacgtgccagctggtgatatcggtgttggtggtcgtgaaattggttacctgttcggtgcttacagatcatacaagaactcctgggaaggtgtcttaaccggtaagggtttgaactggggtggttctttgatcagaccagaagccactggttacggtttagtttactatactcaagctatgatcgactatgccacaaacggtaaggaatctttcgaaggtaagcgcgtcaccatctctggtagtggtaacgttgctcaatacgctgccttgaaggttattgagctaggtggtactgtcgtttccctatctgactccaagggttgtatcatctctgaaactggtatcacctccgaacaagtcgctgatatttccagtgctaaggtcaacttcaagtccttggaacaaatcgtcaacgaatactctactttctccgaaaacaaagtgcaatacattgctggtgctcgtccatggacccacgtccaaaaggtcgacattgctttgccatgtgccacccaaaatgaagtcagcggtgaagaagccaaggccttggttgctcaaggtgtcaagtttattgccgaaggttccaacatgggttccactccagaagctattgccgtctttgaaactgctcgttccaccgccactggaccaagcgaagctgtttggtacggtccaccaaaggctgctaacttgggtggtgttgctgtttctggtttagaaatggcacaaaactctcaaagaatcacatggactagcgaaagagttgaccaagagttgaagagaattatgatcaactgtttcaatgaatgtatcgactatgccaagaagtacactaaggacggtaaggtcttgccatctttggtcaaaggtgctaatatcgcaagtttcatcaaggtctctgatgctatgtttgaccaaggtgatgtattttaa

SEQ ID NO: 12 - DNA, 2709 bp, Saccharomyces cerevisiae
atggagcaaatcaattcgaacagtagaaaaaagaagcaacaattggaagtattcaaatattttgcaagtgtccttacaaaagaggacaagcctattagtatcagtaatggtatgttagatatgccgacagtgaactccagtaaactcacagcaggaaatgggaaacctgacacggagaagcttacaggagaactaattttaacatacgacgatttcattgaactgatatctagctcaaagactatttattcgaagtttacggaccattcgttcaatttgaaccagatacccaagaacgttttcgggtgtattttcttcgctattgatgaacaaaacaagggatatctgacgcttaatgattggttttattttaataatttattagaatatgataattatcatctcattattctatatgagttctttaggaaatttgatgtagagaatttgaaggcaaaacaaaaaaaagagcttggtagttcgtcgtttaatttaaaggctgcagatgatcgaattaagtcaattaattatggtaacagatttctaagctttgatgatcttcttttgaatctgaaccaattcaaagatactatccgcctgttgcacgaatctattgatgataattttgttaaagataacaaattactacttgattggaatgactttcgatttctgaaattttacaaatgttatcatgaaaatgaagagtatttgagtttaaactctctggtcacgattttacaaaatgatcttaagaatgaaaaaatatttataggttttgataggttggcacagatggactcacaagggcatcgtttagccctaagcaaaaatcaactcacctatcttctaaggttattttactctcacagggtgtctgcagatatattttcctccttgaatctatcaaacaccgaattactaaaagcggacaataattccattccgtacaatgtattcaaggatatattttatttatttcaaaattttgacctactgaaccaaatatttcacaagtatgttactgaaaataatttgaatgagcaggatattagggaacaaatagttactaaaaatgactttatgacagttttaaacgcccagtataacaaagtaaacaatatcattgagttctctccttcccaaatcaacctactattttctatcgtcgcaaattcaaaggaaaacagaagattaagaaagagaaatcaagatcgagatgacgagctattaaatgatcaccattatgattcagatattgatttttttatccataatgagtatttgcatggagtaagcagatccagaaaaaatttagaaagttttaatgactattatcatgatctctcggatggatttgaccaagactctggtgttaaaaaagcttcaaaagcgagtactggcttgtttgaatctgtatttggaggtaaaaaagataaagcaacgatgcgttctgacttaacaattgaagatttcatgaaaattttgaacccaaattacctgaacgacttagttcaccaaatggaattgcaaaaaaatcaaaatgagtcattgtatattaattactacttttatccaattttcgattcgttgtacaatttctccttgggttctattgcgggttgtattggtgcaactgtagtatacccaatagactttataaaaacaaggatgcaagcccaaagatctttagcccaatacaaaaactcaattgattgtttgttgaagattatatcccgcgaaggaataaaaggtctctactctggcttagggccacaattaataggagttgctcctgaaaaggcgataaaattgactgtcaatgattttatgagaaacaggttgactgataaaaacggcaagctaagcctttttcctgaaattatttctggcgcttcagctggtgcatgtcaagttatatttactaatccgttagagattgtaaaaattaggctacaggtccaatccgactatgttggtgaaaacatacaacaagccaatgaaactgccactcaaatagtcaaaaaattaggactgaggggcttgtacaatggtgtagccgcatgtttaatgagagatgttccattctctgctatttattttcccacttatgcacatttaaaaaaagatctctttgattttgatccaaatgataaaacaaagaggaatcgattaaaaacatgggagcttttaactgccggtgccattgctggtatgccagctgccttcttgactactccttttgatgttataaaaacaaggctccagatagatcctcgaaaaggtgagacaaagtataacggtatatttcatgctatccgaactatcttaaaggaagagagctttagaagctttttcaaaggtggtggagcccgtgtcctaagaagttctccccaatttgggttcactctggccgcctatgaattattcaagggctttattccctcccccgataacaaattaaaaagcagagagggtaggaagagattttgtatcgatgacgacgcaggcaatgaagagacagtagttcatagtaacggtgaactcccacagcaaaagttttactctgatgatagaaaacatgccaattattactataaaagctgtcaaattgcgaaaacattcattgatttggacaataacttttctaggtttgactcttcagtttataaaaactttcaagagcacctaagaagcattaacgggtga

SEQ ID NO: 13 - DNA, 879 bp, Saccharomyces cerevisiae
atggaggacagtaaaaagaaaggattaatagaaggcgctatactcgatataataaacggttccattgcaggcgcctgtggtaaggtgatcgagtttcctttcgatactgtgaaagtcaggttgcaaacacaagcatccaacgtgttcccaacaacatggtcttgtataaaatttacttaccaaaatgaaggaatagcacgagggttttttcaaggcattgcttcacctttagttggagcatgtctggagaacgcgacattatttgtgtcttataaccaatgttctaaatttttagaaaaacatacaaacgtttccccgttggggcaaatcctgatctctggtggagtagcgggttcatgtgctagtttagttttgacacccgtggagctggtgaagtgtaagttgcaggttgcgaacttacaagttgcatcagctaaaacgaaacatacaaaggtgttgcctacaataaaagcaattataactgagagaggattggcaggattgtggcaagggcaatcgggcacttttattcgagaaagcttcggtggtgttgcctggtttgcaacctacgaaatagttaagaagtcgttgaaagataggcactcccttgatgacccaaaaagagatgaaagtaagatatgggaactacttattagtggagggagcgctggattggcattcaacgccagtatttttcctgcggatactgtgaaatcagtaatgcaaactgaacatataagcctcaccaatgcggtgaagaagatatttggcaaatttggactaaagggtttttatcgaggactgggtataaccctttttagggcagtaccagcaaacgctgcagttttttacatctttgagactctttctgcactttaa

SEQ ID NO: 14 - DNA, 1332 bp, Escherichia coli
atggtaaaggaacgtaaaaccgagttggtcgagggattccgccattcggttccctatatcaatacccaccggggaaaaacgtttgtcatcatgctcggcggtgaagccattgagcatgagaatttctccagtatcgttaatgatatcgggttgttgcacagcctcggcatccgtctggtggtggtctatggcgcacgtccgcagatcgacgcaaatctggctgcgcatcaccacgaaccgctgtatcacaagaatatacgtgtgaccgacgccaaaacactggaactggtgaagcaggctgcgggaacattgcaactggatattactgctcgcctgtcgatgagtctcaataacacgccgctgcagggcgcgcatatcaacgtcgtcagtggcaattttattattgcccagccgctgggcgtcgatgacggcgtggattactgccatagcgggcgtatccggcggattgatgaagacgcgatccatcgtcaactggacagcggtgcaatagtgctaatggggccggtcgctgtttcagtcactggcgagagctttaacctgacctcggaagagattgccactcaactggccatcaaactgaaagctgaaaagatgattggtttttgctcttcccagggcgtcactaatgacgacggtgatattgtctccgaacttttccctaacgaagcgcaagcgcgggtagaagcccaggaagagaaaggcgattacaactccggtacggtgcgctttttgcgtggcgcagtgaaagcctgccgcagcggcgtgcgtcgctgtcatttaatcagttatcaggaagatggcgcgctgttgcaagagttgttctcacgcgacggtatcggtacgcagattgtgatggaaagcgccgagcagattcgtcgcgcaacaatcaacgatattggcggtattctggagttgattcgcccactggagcagcaaggtattctggtacgccgttctcgcgagcagctggagatggaaatcgacaaattcaccattattcagcgcgataacacgactattgcctgcgccgcgctctatccgttcccggaagagaagattggggaaatggcctgtgtggcagttcacccggattaccgcagttcatcaaggggtgaagttctgctggaacgcattgccgctcaggcgaagcagagcggcttaagcaaattgtttgtgctgaccacgcgcagtattcactggttccaggaacgtggatttaccccagtggatattgatttactgcccgagagcaaaaagcagttgtacaactaccagcgtaaatccaaagtgttgatggcggatttagggtaa

SEQ ID NO: 15 - DNA, 777 bp, Escherichia coli
atgatgaatccattaattatcaaactgggcggcgtactgctggatagtgaagaggcgctggaacgtctgtttagcgcactggtgaattatcgtgagtcacatcagcgtccgctggtgattgtgcacggcggcggttgcgtggtggatgagctgatgaaagggctgaatctgccggtgaaaaagaaaaacggcctgcgggtgacgcctgctgatcagatagacattatcaccggagcactggcgggaacggcaaataaaaccctgttggcatgggcgaagaaacatcagattgcggccgtaggtttgtttctcggtgacggcgacagcgtcaaagtgacccagcttgatgaagagttaggtcatgttggactggcgcagccaggttcgcctaagcttatcaactccttgctggagaacggttatctgccggtggtcagctccattggcgtaacagacgaagggcaactgatgaacgtcaatgccgaccaggcggcaacggcgctggcggcaacgctgggcgcggatctgattttgctctccgacgtcagcggcattctcgacggcaaagggcaacgcattgccgaaatgaccgccgcgaaagcagaacaactgattgagcagggcattattactgacggcatgatagtgaaagtgaacgcggcgctggatgcggcccgcacgctgggccgtccggtagatatcgcctcctggcgtcatgcggagcagcttccggcactgtttaacggtatgccgatgggtacgcggattttagcttaa

SEQ ID NO: 16 - DNA, 1074 bp, Corynebacterium glutamicum
atgataatgcacaatgtctatggtgttacaatgactattaaggtcgcaatcgcaggtgcctcaggttacgcaggtggtgaaatcttgagattgttattgggtcatccagcatatgcctctggtgaattagaaataggtgcattgaccgctgcatccactgccggtagtacattgggtgaattgatgccacatattcctcaattagctgatagagttatacaagacactacagctgaaacattggcaggtcatgatgttgtctttttaggtttgccacacggtttctcagcagaaatagccttacaattgggtcctgatgtcacagtaatcgattgtgccgctgactttagattacaaaatgcagccgactgggaaaaattctatggttccgaacatcaaggtacctggccatacggtattccagaaatgcctggtcacagagaagccttgagaggtgctaagagagttgcagtcccaggttgctttcctacaggtgctaccttagcattattgccagccgttcaagctggtttgatcgaacctgatgtatctgtagtttcaattaccggtgtttccggtgcaggtaaaaaggctagtgttgccttattgggttctgaaactatgggttcattgaaggcatacaacacctcaggtaaacatagacacactccagaaatcgctcaaaacttgggtgaagtttctgacaaaccagtaaaggtttcattcacacctgttttagctccattgcctagaggtattttaaccactgctacagcacctttgaaagaaggtgtcaccgccgaacaagccagagctgtttacgaagaattctacgctcaagaaactttcgtccatgtattaccagaaggtgcccaacctcaaacacaagctgttttgggttccaacatgtgtcacgttcaagtcgaaattgatgaagaagctggtaaagtattggttactagtgcaatcgacaatttgactaagggtacagcaggtgctgcagttcaatgcatgaacttatctgtcggttttgatgaagccgctggtttgccacaagtcggtgtagctccttaa

SEQ ID NO: 17 - DNA, 1176 bp, Corynebacterium glutamicum
atgtctacattggaaacctggcctcaagtcatcatcaacacatacggtactcctcctgtcgaattggtctctggtaaaggtgctacagtaaccgatgaccagggtaacgtttacatcgatttgttggctggtatagcagttaacgccttgggtcatgctcacccagcaataatcgaagctgtaactaaccaaataggtcaattgggtcatgtttctaacttatttgcatcaagacctgttgtcgaagttgccgaagaattaattaagagattctctttggatgacgcaacattagctgcacaaaccagagttttcttttgtaattcaggtgcagaagccaacgaagccgcttttaaaatcgctagattgacaggtagatccagaattttagcagccgttcatggtttccacggtagaaccatgggtagtttggcattaactggtcaaccagataagagagaagcatttttgccaatgccttccggtgttgaattctatccttacggtgacactgactatttgagaaaaatggtcgaaaccaatccaactgatgtagctgcaatctttttagaacctattcaaggtgaaacaggtgtagttccagcccctgaaggtttcttgaaggctgttagagaattgtgtgatgaatacggtatcttgatgatcactgacgaagtacaaacaggtgttggtagaaccggtgactttttcgcacatcaacacgatggtgtcgtaccagacgttgtcactatggctaaaggtttgggtggtggtttacctattggtgcctgcttggctacaggtagagccgctgaattaatgaccccaggtaaacatggtactacatttggtggtaaccctgttgcttgtgcagccgctaaagcagtcttgtcagtagttgatgacgcattttgcgccgaagttgctagaaagggtgaattattcaaggaattgttggctaaggttgatggtgtcgtagacgtcagaggtagaggtttgatgttaggtgttgtcttggaaagagatgtcgcaaagcaagccgtattggacggttttaaacacggtgttattttaaatgctccagcagataacatcattagattgactccacctttagtcataacagatgaagaaattgccgacgctgttaaagcaattgccgaaacaatagcttaa

SEQ ID NO: 18 - DNA, 1167 bp, Corynebacterium glutamicum
atggccgaaaaaggtataacagctccaaaaggtttcgttgcctctgctactacagccggtatcaaggcttcaggtaatccagatatggcattggttgtcaaccaaggtcctgaattttctgctgcagccgttttcactagaaatagagtctttgctgcacctgttaaagtctctagagaaaacgttgctgatggtcaaattagagctgtcttgtataatgctggtaatgcaaacgcctgtaacggtttacaaggtgaaaaggatgcaagagaatccgtaagtcatttggcccaaaatttgggtttagaagattccgacatcggtgtttgcagtacaggtttgattggtgaattgttgccaatggataagttgaacgctggtatcgaccaattgaccgccgaaggtgctttaggtgacaacggtgccgctgcagccaaagctatcatgaccactgataccgttgacaaggaaactgtagtttttgcagatggttggacagtaggtggtatgggtaaaggtgttggtatgatggcaccttcattggccaccatgttagtatgtttaacaaccgatgcctccgttactcaagaaatggctcaaattgctttggcaaatgccaccgctgtcactttcgacacattagatatagacggttctacatcaaccaacgatactgttttcttgttagcatctggtgcctcaggtatcactccaacacaagatgaattgaatgacgctgtttacgctgcatgctctgatattgccgctaaattacaagcagacgccgaaggtgttacaaagagagtagcagttaccgtcgtaggtactacaaataacgaacaagctattaatgcagccagaacagttgcaagagataacttgtttaaatgtgccatgttcggttctgacccaaattggggtagagtcttagctgcagttggtatggctgatgcagacatggaacctgaaaagatatccgtctttttcaacggtcaagctgtatgcttggatagtactggtgctcctggtgcaagagaagtcgacttgtctggtgctgatattgacgttagaatagatttgggtacttcaggtgaaggtcaagcaacagttagaaccactgatttgtcctttagttacgtcgaaattaattccgcttactcttcataa

SEQ ID NO: 19 - DNA, 1017 bp, Saccharomyces cerevisiae
atgtcaaccacagcatccacgccttcatctttacgtcatttgatttctataaaagatctttctgatgaagaattcagaatcttagtacaaagagctcaacatttcaagaatgtttttaaagcaaataaaacgaatgatttccaatccaaccatctgaaactattgggtagaactatagccttaatatttactaaaagatcaactagaacgagaatttcgaccgaaggtgcagccaccttctttggtgcccaaccgatgtttttaggtaaagaggatattcagcttggtgtcaatgaatcattttacgataccaccaaggttgtatcatctatggtttcatgtatttttgcccgtgtgaacaaacatgaagacatacttgctttttgcaaggattcctctgtaccgatcatcaactctctatgtgacaaattccaccctttgcaagcaatttgtgatcttttaacaataatcgaaaacttcaatatatctctagatgaagtaaataagggaatcaattcaaaattgaagatggcatggattggtgatgccaataatgtcataaatgatatgtgcatcgcatgtctgaaattcggtataagtgtcagtatttccactccccccggtattgaaatggattccgatattgtcgatgaagcaaagaaagttgctgagagaaacggtgcgacatttgaattaacacacgactctttaaaggcctccaccaatgccaatatattagtaaccgatactttcgtttccatgggtgaagaatttgcgaaacaggccaagctgaaacaattcaaaggttttcaaatcaatcaagaacttgtctctgtggctgatccaaactacaaatttatgcattgtctgccaagacatcaagaagaagttagtgatgatgtcttttatggagagcattccatagtctttgaagaagcagaaaacagattatatgcagctatgtctgccattgatatctttgttaataataaaggtaatttcaaggacttgaaataa

SEQ ID NO: 20 - DNA, 1275 bp, Saccharomyces cerevisiae
atgtccgaagctaccctctcctccaagcaaaccattgaatgggaaaacaaatactccgcccacaactaccaccccttgcccgtcgtttttcacaaggctaagggcgcacatgtgtgggacccggagggtaagctgtacctcgacttcctgagcgcttattctgccgtcaaccagggccattgccatcctcacatcatcaaggctttgacggagcaagcacaaacactaacattgtcctccagagcgttccacaacgatgtttacgcgcaattcgccaagttcgtgaccgaattcttcgggttcgaaaccgttttgcccatgaacaccggtgcagaagccgtggaaactgctttgaagttggccagaagatgggggtacatgaagaagaacatccctcaagataaagccatcattctgggtgccgagggtaacttccacgggagaaccttcggtgctatcagtttgagtaccgactacgaggactccaagttgcatttcgggcctttcgtgcctaacgttgccagtggtcactccgtgcacaagatcagatacggccacgcagaagatttcgtccctatcttggaatctcctgaaggtaagaacgttgccgccatcattctagagccaattcagggtgaagccggtatcgtcgtgccccccgcagactacttcccaaaggtctccgcattatgccgtaagcacaacgtcctattgatcgttgacgaaattcaaaccggtatcggtagaaccggtgagttgctttgctacgaccactacaaggcagaggccaagcctgatattgttttgttaggtaaggctctctcaggtggtgttcttcccgtctcatgtgttctgtcttctcacgacatcatgtcttgctttaccccaggatctcacggttctactttcggcggtaatcctttggcttcccgcgttgccatcgccgccctcgaggtcatccgcgacgagaagctgtgccaaagagccgcccaactgggtagctctttcatcgcccaattgaaagctctccaagccaaatctaacggtataatctctgaggtgcgtggtatgggactgcttaccgccatcgtaatcgacccatccaaggccaatggtaagaccgcttgggacttgtgtctattgatgaaggatcacggcctcttggctaagcccacccacgaccacatcatcagattggctcctcctttggtcatctccgaagaggacttgcaaaccggtgtcgaaaccattgccaagtgtatcgatctgttataa

SEQ ID NO: 21 - DNA, 1401 bp, Saccharomyces cerevisiae
atgtctagtactcaagtaggaaatgctctatctagttccactactactttagtggacttgtctaattctacggttacccaaaagaagcaatattataaagatggcgagacgctgcacaatcttttgcttgaactaaagaataaccaagatttggaacttttaccgcatgaacaagcgcatcctaaaatatttcaagcgctcaaggctcgtattggtagaattaataatgaaacgtgcgaccccggtgaggagaactcgtttttcatatgcgatttgggagaagtcaagagattattcaacaactgggtgaaggagcttcctagaattaagccattttatgccgtcaaatgtaatcctgataccaaggttttgtcattattagcagagttgggcgttaatttcgattgcgcttccaaagtggaaattgacagagtattatcgatgaacatctcgccggatagaattgtttacgctaatccttgtaaagtagcatctttcattagatatgcagcttcaaaaaatgtaatgaagtctacttttgacaatgtagaagaattgcataaaatcaaaaagtttcatcctgagtctcagttgttattaagaatcgctaccgatgactctaccgctcaatgtcgactttccaccaaatatggctgtgaaatggaaaacgtagacgttttattaaaggctataaaggaactaggtttaaacctggctggtgtttctttccacgtcggttcaggcgcttctgattttacaagcttatacaaagccgttagagatgcaagaacggtatttgacaaagctgctaacgaatacgggttgccccctttgaagattttggatgtaggtggtggatttcaatttgaatccttcaaagaatcaactgctgttttgcgtctagcgctagaggaatttttccctgtaggttgtggtgttgatataattgcagagcctggcagatactttgtagctacagcgttcactttggcatctcatgtgattgcgaagagaaaactgtctgagaatgaagcaatgatttacactaacgatggtgtatacgggaacatgaattgtattttattcgatcatcaagagccccatccaagaaccctttatcataatttggaatttcattacgacgattttgaatccactactgcggtcctcgactctatcaacaaaacaagatctgagtatccatataaagtttccatctggggacccacatgtgatggtttggattgtattgccaaagagtattacatgaagcatgatgttatagtcggtgattggttttattttcctgccctgggtgcctacacatcatcggcggctactcaattcaacggctttgagcagactgcggatatagtatacatagactctgaactcgattga

SEQ ID NO: 22 - DNA, 879 bp, Saccharomyces cerevisiae
atgtatgaagtaatacagaaaaggaaaacaaaaataataaacgttttacagagtcctgaactcatgaggctcatagaggacccatcaaatctgggtatttctttacattttccagtaagttcactgctaaaaagtaataagtgcacaccaatgcctaaactttctacgtatagtttggctagtgggggatttaaggattggtgcgcggacatccctctagacgttccaccagagattgatatcatcgatttttactgggatgttattttatgcatggaatctcaattcatattagattacaatgttccgtcaaaaaataaggggaacaatcagaagtctgttgctaagctgttgaaaaataagcttgtaaacgatatgaaaactacgttaaaaagactaatttataatgaaaataccaagcaatataaaaataataatagccacgatggttacaattggagaaaactaggctcgcagtatttcatactgtatcttcccctatttacgcaggaactgatttggtgtaaacttaatgaaaactatttccatgttgtattaccatctttactgaatagtaggaacgttcatgataaccacagtacctatataaataaagattggttacttgcccttttagagctaacttccaacctgaaccaaaacttcaaattcgaatacatgaaattgagattgtatattttaagagatgatttaattaataatggtttggatcttttgaaaaatcttaactgggtcggtgggaaactgattaaaaatgaagatagagaagtcttgttgaactcgaccgatttagctacggattctatttctcatttattaggtgatgaaaactttgttattttagagtttgaatgctaa

SEQ ID NO: 23 - DNA, 1191 bp, Saccharomyces cerevisiae
atgactgtcaccataaaagaattgactaaccacaactacattgaccacgaactatcagccactttagactcaacggatgcgttcgagggtcccgagaagttgctggaaatctggttcttccctcacaagaagtccatcacgaccgaaaagacattaagaaatattggcatggatagatggatcgagattttgaaattagtgaaatgcgaagttctttccatgaagaagactaaagaactggatgcctttttgttgagtgagtcttccctcttcgtcttcgatcacaaattgacgatgaagacgtgcggtactacaaccacattgttctgtctcgaaaagcttttccagatcgttgagcaagagttatcgtgggctttccgcacaacacaagggggcaagtacaaaccatttaaagtgttttattctagacgatgtttccttttcccctgtaagcaagccgctatccatcaaaactgggctgacgaagtcgactatttgaacaaatttttcgacaatggtaaaagttattccgtgggaagaaatgacaagagcaaccactggaacctgtacgtcaccgagacggaccgctccacacctaagggaaaggagtacatcgaggatgacgacgaaactttcgaagtactgatgacggagctggacccagaatgcgctagtaagtttgtttgcgggcctgaggcatccacaaccgctctcgtggagccaaacgaagataagggccacaacctcggctaccaaatgactaaaaatacaaggcttgacgaaatatatgtcaactcggcccaagactccgatttatcatttcaccacgatgcatttgcgttcacgccatgtggatactcatccaatatgattctcgctgaaaaatactattacaccctgcacgtgactccggaaaagggttggtcttacgcctctttcgaaagtaacatacccgtatttgacatttcccaagggaagcaagacaacttggacgttcttctacatattctgaacgtttttcaaccaagagagttctcgatgaccttttttaccaaaaattatcagaaccaatccttccaaaaactactaagcatcaacgagtcactgcccgactacatcaagttagacaaaattgtttatgatctggacgactaccaccttttctatatgaaattgcagaagaaaatatga

SEQ ID NO: 24 - DNA, 882 bp, Saccharomyces cerevisiae
atggcacaagaaatcactcacccaactattgtagacggctggttcagagaaatttctgataccatgtggccaggccaggccatgactttaaaagtggagaaagttttacaccatgagaagtcaaaatatcaagacgttttgatcttcaaatccactacatatggtaatgttctagttttagataatgtaattcaagccaccgaaagggatgaatttgcctaccaagaaatgattgcccatcttgccttgaattcccatccaaatcctaagaaggttcttgttattggtgggggtgatggtggtgttttgagagaggttgtcaagcatgattccgttgaggaagcctggttatgtgacattgatgaagctgttattagactatcaaaggagtacctaccagaaatggctgcctcttattctcacccaaaggttaagacccacattggtgatggtttccaatttttaagagattaccaaaacacatttgacgtaatcattactgactcttctgacccagaaggtccagctgaaacccttttccaaaaggaatatttccaattgttgaacagtgcgttgacagaaaagggtgtaatcactacacaagcagaaagtatgtggattcacttgccaatcattaaggacttaaagaaagcctgttctgaagttttcccagttgcagaatactctttcgttactattccaacttacccaactggtacgattggttttatggtttgctccaaagataaaacttgcaatgtcaagaagccactacgtgaaatctctgatgagaaggaggctgaattatacagatactataacaagaaaattcacgaagcttcctttgttctaccaacctgggcagccaaggaattaaattag

SEQ ID NO: 25 - DNA, 1527 bp, Saccharomyces cerevisiae
atgaatacagtttcaccagccaaaaaaaaggttattataattggtgccggtattgctgggcttaaagctgcatctacgctacaccaaaacggtattcaagattgtcttgttcttgaggccagagatcgggtcggtggtaggttgcaaactgtcacaggctatcaaggtcggaaatatgatataggtgctagctggcaccatgatacgttgacaaaccctttatttttggaagaggctcaactgagtttgaatgatgggagaacgaggtttgtttttgatgacgataattttatttatatcgacgaagaacgtggaagggtagaccatgacaaggaactgcttcttgaaattgtggacaatgaaatgagcaaattcgcagagttagaattccatcaacacttaggagtttcagattgctccttttttcaattagtaatgaaatacttactacaaagacgccaatttctcacaaatgaccaaataagatatttgccacaactctgtcgatatctggaattgtggcacggcttagattggaagcttttgagtgccaaggatacatacttcggtcaccaaggaaggaacgcctttgctttgaactatgattctgtggttcaaagaattgctcaaagctttcctcaaaattggttaaagctaagttgtgaagtgaaatcaattacacgagaaccttcaaaaaatgtgacagtgaactgtgaagatggtactgtgtacaatgctgattatgttattattacagtacctcaaagtgtattgaatttgtctgtacaacctgaaaaaaatttacggggaagaatagaatttcaaccacccttgaaaccagtgattcaagatgcttttgacaagatccattttggagcgctaggtaaagtaatttttgagtttgaagaatgttgttggtcgaacgaaagttcaaaaattgtaactttggctaactctaccaatgaatttgtcgaaatagtacgtaatgcggaaaatttagatgaattagactctatgctagaaagggaagattctcaaaagcatacgagtgttacttgttggagccagcctttatttttcgtaaatttgtcaaaaagcacaggagtagcaagctttatgatgttgatgcaggcaccgcttacaaatcacatagaatccattagagaagataaagagcgtctttttagttttttccaacctgtgctgaacaagattatgaagtgtctagattctgaggatgtcatcgacggaatgaggccgatagaaaacattgcaaacgctaataaaccagtcttaagaaacatcatcgttagcaactggacacgcgatccttactcacgcggtgcttattcggcctgttttccaggagatgatccagttgatatggttgttgcaatgtctaatggtcaagactcccgcataagatttgcaggcgaacatactatcatggacggcgccggctgtgcctatggtgcttgggaaagcggaagacgggaggcgactcgaatctctgacttactgaaatag

SEQ ID NO: 26 - DNA, 1014 bp, Saccharomyces cerevisiae
atgaacaggattaagaatacattttctgttgctaagagattaaaactaagcaaagttatgacgaactcagaattaccgagcatattcgaaggaactgttgatttagggattattggtggtacaggtttatataaccttgactgtctggagcccatcgctttgcttccacccatggtaacaccatggggtaccacatcgtctcctgtcacaatctctcagttcgtaggaactaacagccactttcacgttgcgttcatagccagacacggtattaaccacgaatacccacccactaaagtcccatttagagcaaacatggcggccttaaagaacttaaattgtaaagccgttctttcttttagtgccgtggggtctttacaaccccatataaagcctagagattttgtgttaccacagcaaataatcgacagaactaaaggcataagacattcttcatatttcaacgatgaaggcttggtaggtcacgttggtttcggacagccgttctctcaaaaattcgcagagtatatctatcaattcaagaacgagataacaaatcctgaatccgaagaaccgtgccatttgcattacgacaaggatatgaccgttgtgtgtatggaaggcccacaattctccacgcgcgctgaatccaagatgtacagaatgtttggtggccatgttattaacatgagtgttattccagaagccaaattggcgcgtgagtgtgagctgccttaccagatgatttgtatgtctaccgattacgacgcatggagagatgaggcagaacctgttaccgtagaaaccgttattggtaatttgacgaataatgggcgcaatgcaaatattttagcttctaagatcatcgtctcaatggccaaggaaatcccagagttcatgcatactggcgatgggctgcgcggttccatcaagaaatctatctctaccaaaccagaggctatgtccaaggaaaccttagaaagactaagatacttatttccaaactattggtaa

SEQ ID NO: 27 - DNA, 1131 bp, Saccharomyces cerevisiae
atgaccttggcacccctagacgcctccaaagttaagataactaccacacaacatgcatctaagccaaaaccgaacagtgagttagtgtttggcaagagcttcacggaccacatgttaactgcggaatggacagctgaaaaagggtggggtaccccagagattaaaccttatcaaaatctgtctttagacccttccgcggtggttttccattatgcttttgagctattcgaagggatgaaggcttacagaacggtggacaacaaaattacaatgtttcgtccagatatgaatatgaagcgcatgaataagtctgctcagagaatctgtttgccaacgttcgacccagaagagttgattaccctaattgggaaactgatccagcaagataagtgcttagttcctgaaggaaaaggttactctttatatatcaggcctacattaatcggcactacggccggtttaggggtttccacgcctgatagagccttgctatatgtcatttgctgccctgtgggtccttattacaaaactggatttaaggcggtcagactggaagccactgattatgccacaagagcttggccaggaggctgtggtgacaagaaactaggtgcaaactacgccccctgcgtcctgccacaattgcaagctgcttcaaggggttaccaacaaaatttatggctatttggtccaaataacaacattactgaagtcggcaccatgaatgcttttttcgtgtttaaagatagtaaaacgggcaagaaggaactagttactgctccactagacggtaccattttggaaggtgttactagggattccattttaaatcttgctaaagaaagactcgaaccaagtgaatggaccattagtgaacgctacttcactataggcgaagttactgagagatccaagaacggtgaactacttgaagcctttggttctggtactgctgcgattgtttctcccattaaggaaatcggctggaaaggcgaacaaattaatattccgttgttgcccggcgaacaaaccggtccattggccaaagaagttgcacaatggattaatggaatccaatatggcgagactgagcatggcaattggtcaagggttgttactgatttgaactga

SEQ ID NO: 28 - DNA, 564 bp, Saccharomyces cerevisiae
atgtctatagcaagttatgcccaagagttgaagttggctttacatcaatatccaaacttccctagtgaaggcattctcttcgaagatttcttacccattttcaggaacccaggtcttttccagaagttgatcgatgctttcaaactgcatttagaagaagcttttccagaagttaaaattgattatatcgtcgggttggaatcccgtgggttcttgttcggaccaactttagctttggccctaggtgttggtttcgttccagtcaggaaggcaggtaagctacctggcgaatgttttaaggctacgtacgaaaaggagtacggttctgatctttttgagatacagaaaaacgctattccagcaggttccaacgttatcattgttgatgacattattgccactggtggttctgctgctgcagccggcgaattagttgaacaactcgaagccaaccttttggaatataactttgttatggagttggatttcttgaaaggcaggagtaagttgaatgctccagtgttcactttactgaacgctcaaaaggaagcgttgaaaaaatga

SEQ ID NO: 29 - DNA, 1491 bp, Saccharomyces cerevisiae
atgtcaatgagtaatattgttgtttttggaggggactcgcaccccgagttagttactaagatctgtgaaaatttggacattcacccatcgaaagtagaattagggaagttttctaatggggaaacgaacattgctcttcgcgaatctgttcgtgaaaaggatgtatatatcatccagagtggttgtggccaggtgaacgacacgttcatgcagttgctgattttaatcagtgcctgcaagtccgcttctgcctcgagggttacagccgtaatgccatatctctgctactcgagacagccagatattccatatactgccaagggtgctcccataatttccaagcctaaagaaaactatacttttgaatcgcatccaggcacacccgtgtcatcttctttaatgacgcaaagaccaggtgctgagagctcgttgaagagtttggatagtgcaatacgatcaactatcaacttagaaaatcctcaacctatcagaacaccaaacagcagtgctacggcgaataacaatttcgacatcaagaagacgctttctttttcaagaattcctatgattcccggtggtaagttacaaaatacaagcaatagcacggacgctggtgaattgttcaacgctcaaaatgcaggctataagctatgggtagtacaagccggtactttgattgctcatttgttgagtgctgcaggtgctgaccatgtgatcacaatggatttgcacgatccacagttccctgggttttttgacattccagtggataatctctactgtaaacccattgcacaaaactacatccagcatcgcattccagattatcaggatgctgtgatcgtttctccagatgctggtggtgcaaagagagctacggctattgcagacgccttggaattgtccttcgccctaattcataaagaaagaagatctcagttattgaagggccctccagatgcgacgttaacctctggtggttcgttaccagtatctccaaggccattagttactactttggtttcctcccaaaatactacttcttcaggtgccactggggttgcggcccttgaaatgaagaaaacaacttcaacatcttccacctcgtcgcaatcttctaattcgtccaagttcgttcaaactaccatgcttgttggcgatgttagaaacaaggtgtgtattatagtcgacgacttggtggatacttcatacactattacaagagctgcgaaattgttgaaggatcaaggatctaccaaagtttatgccttaataacgcacggtgttttttccggtgatgcgctagaaagaatcggccaaagtagtatagataagttgatcatttctaacacggttcctcaagatagaacactacagtacctaggtaaggacagagtggatgttattgatgtctcctgcataatcggtgaagcaattagaagaatccataacggtgaatccatttctatgttgttcgagcatggatggtag

SEQ ID NO: 30 - DNA, 1197 bp, Leishmania infantum
gttgtttctagaaaaacaatgtctgttcactctatcttgttctcttctgaacacgttactgaaggtcacccagacaagttgtgtgaccaagtttctgacgctgttttggacgcttgtttggctggtgacccattctctaaggttgcttgtgaatcttgtgctaagactggtatggttatggttttcggtgaaatcactactaaggctgttttggactaccaaaagatcgttagaaacactatcaaggacatcggtttcgactctgctgacaagggtttggactacgaatcttgtaacgttttggttgctatcgaacaacaatctccagacatctgtcaaggtttgggtaacttcgactctgaagacttgggtgctggtgaccaaggtatgatgttcggttacgctactgacgaaactgaaactttgatgccattgacttacgaattggctagaggtttggctaagaagtactctgaattgagaagagacggttctttggaatgggctagaccagacgctaagactcaagttactgttgaatacgactacgacactagagaaggtaagcaagttttgactccaaagagagttgctgttgttttgatctctgctcaacacgacgaacacgttactaacgacaagatctctgttgacttgatggaaaaggttatcaaggctgttatcccagctaacatgttggacgctgaaactaagtactggttgaacccatctggtagattcgttagaggtggtccacacggtgacgctggtttgactggtagaaagatcatcgttgacacttacggtggttggggtgctcacggtggtggtgctttctctggtaaggacccatctaaggttgacagatctgctgcttacgctgctagatggatcgctaagtctatcgttgctggtggtttggctagaagatgtttggttcaattggcttacgctatcggtgttgctgaaccattgtctatgcacgttgaaacttacggtactggtaagtacgacgacgctaagttgttggaaatcgttaagcaaaacttcaagttgagaccatacgacatcatccaagaattgaacttgagaagaccaatctactacgaaacttctcgtttcggtcacttcggtagaaaggacgaattgggtactggtggtttcacttgggaagttccaaagaagatggttgaataa

SEQ ID NO: 31 - DNA, 1044 bp, Saccharomyces cerevisiae
atggtttctgtggagtttttacaggagttaccaaaatgtgagcatcacttgcatttggaaggtactctagaacctgacctattgttcccattagctaaaagaaacgatataattctacctgaaggttttcctaaatcggtcgaggaattaaacgaaaagtataagaagtttcgtgatctgcaggatttcttagattactattatattggtactaatgtcttgattagtgaacaagatttctttgatttggcgtgggcctattttaaaaaagttcacaaacaaggcttggtccatgctgaagtgttttacgaccctcagtcacatacatctaggggcatctccatagaaacagtcactaaaggtttccaaagagcttgtgacaaagccttctctgaatttggtattacatccaagctaattatgtgtctgttaagacacattgaaccagaggaatgtttgaaaactatcgaagaagctaccccatttattaaagatggtactatctctgccttaggattagattctgctgagaaaccatttcccccacatttatttgttgaatgttacggaaaggccgcctcattgaataaagatttaaaactaactgcacacgcaggtgaagaaggccccgctcaattcgtctcggatgctttagacttgttgcaagtaacaagaatcgatcacggtatcaacagtcaatacgacgaggagttattggataggttgtcgcgcgaccagaccatgctaactatttgtcctctctccaacgtgaagctacaagtagtccaatccgtttcagagttaccactacaaaagtttcttgacagagatgttccattttctttaaattctgatgaccccgcctattttggtggttatatcttagatgtctacactcaagtttcgaaagatttcccacactgggaccatgaaacatggggtcgtatcgctaagaacgccattaaaggttcatggtgtgacgataaaagaaagaacggtttgttaagtagagtggacgaagtagtcactaaatattcgcattag

SEQ ID NO: 32 - DNA, 1857 bp, Saccharomyces cerevisiae
atgccagagtatacgctactggctgataatataagggagaatatcgttcatttcgatccgaatggtttgtttgataacttgcacaccattgttcatgaagatgacagtcaagagaacgaggaggccgagcatttcaattatgatcaggtgttggataaatcgttattgtcaagaggttctattgtcggtctcggtttaggactaatgagtcccgttttaggaatgtgcactagtatggccattgggctaattaatggtggtccgttaactataatgctaggttttttaatcagtggagtgtgtatatggttttcgtcgctttctcttggtgagattgtttcaaaatttccgatggaactgcatgttgggagtgccatgttggccccggagaaattgaaattagtatgttcgtggtacactggctggttaatgctcatagggaattggactatgagtaccagtattacttttgcaggcgctcaacttaccatttctttgattctgatgacgaactccaacctaatatccgaggcacacttgattttttacacagtcattgtattttacttagttgtgactgttgtaggcctcgtgaatttgaaatttgcaagatttattgaaacaataaacaaagtctgtgtttattggatcatatatgccattatatttattgatattcttctactagtattccacaaaggtaaatttcgatctttgaagtacgcgctatttcactttgataataatctatcagggtataaaagcgcatttctttccttcatcattggattccaacagtctaatttcacgttacaaggtttcagtatgttacctgctttagctgacgaagtcaaagttcctgagaaggatattccacgtggtatgtcgaatgcggtattgttatccgcgttctctggagtcatttttcttataccaataatgttaatcctgccagataatgatttgctttttaccaatcataaggttctaccaatagtgaacatttttacaaaatcgactgattcggtggtcttgtctttttttttagtgctcctaattttaggaaacttactgttttccggaattggctcgattactacatcttctcgtgcggtatatagttttagtcgtgaccaggctataccatactacgataaatggacctacgtcgaaccggattctcagtcaaaagtccccaagaattctgttgtattgagtatgataatatcatactttttaggtctgctagctttgatttcaacggccgcatttaatgcttttataggcgctgcagtgctctgtctttgttctgcgactttcattccgttagtcttggtgctgtttacgagaagaagagctatccgaagcgcgccagtaaaaatcaggtataagtttggttggttcatcaacattgtttctattgtgtggctcttgttatctatggtttctgtttgcctaccaacgcaagtgcctgtaactttcaaaacaatgaattatgctttaatggtgtacgtattctgcattttagttatcactggtctttatttcaaatgggggaagtataattttagattacccttggcagatgacatcaaggctccaattcccagtgatgcggaagaaactgtttttgaactagaggatagcaatgttgaacatactctaaactcgggaaccacagtgaaagagtctgtagaaaataattctgaagaaggtttcatcaaggtgcatcctaaaagtagtacagaaaatccctttgaggaaaatgaggaaaacgtgataaccgattatggtgatgagcaccatacagcagaacaagaatttgatcttgccgatgatcgtagatatgatatatga

SEQ ID NO: 33 - DNA, 2088 bp, Saccharomyces cerevisiae
atgactgtcaccataaaagaattgactaaccacaactacattgaccacgaactatcagccactttagactcaacggatgcgttcgagggtcccgagaagttgctggaaatctggttcttccctcacaagaagtccatcacgaccgaaaagacattaagaaatattggcatggatagatggatcgagattttgaaattagtgaaatgcgaagttctttccatgaagaagactaaagaactggatgcctttttgttgagtgagtcttccctcttcgtcttcgatcacaaattgacgatgaagacgtgcggtactacaaccacattgttctgtctcgaaaagcttttccagatcgttgagcaagagttatcgtgggctttccgcacaacacaagggggcaagtacaaaccatttaaagtgttttattctagacgatgtttccttttcccctgtaagcaagccgctatccatcaaaactgggctgacgaagtcgactatttgaacaaatttttcgacaatggtaaaagttattccgtgggaagaaatgacaagagcaaccactggaacctgtacgtcaccgagacggaccgctccacacctaagggaaaggagtacatcgaggatgacgacgaaactttcgaagtactgatgacggagctggacccagaatgcgctagtaagtttgtttgcgggcctgaggcatccacaaccgctctcgtggagccaaacgaagataagggccacaacctcggctaccaaatgactaaaaatacaaggcttgacgaaatatatgtcaactcggcccaagactccgatttatcatttcaccacgatgcatttgcgttcacgccatgtggatactcatccaatatgattctcgctgaaaaatactattacaccctgcacgtgactccggaaaagggttggtcttacgcctctttcgaaagtaacatacccgtatttgacatttcccaagggaagcaagacaacttggacgttcttctacatattctgaacgtttttcaaccaagagagttctcgatgaccttttttaccaaaaattatcagaaccaatccttccaaaaactactaagcatcaacgagtcactgcccgactacatcaagttagacaaaattgtttatgatctggacgactaccaccttttctatatgaaattgcagaagaaaataggatctggttctggttctatggcacaagaaatcactcacccaactattgtagacggctggttcagagaaatttctgataccatgtggccaggccaggccatgactttaaaagtggagaaagttttacaccatgagaagtcaaaatatcaagacgttttgatcttcaaatccactacatatggtaatgttctagttttagataatgtaattcaagccaccgaaagggatgaatttgcctaccaagaaatgattgcccatcttgccttgaattcccatccaaatcctaagaaggttcttgttattggtgggggtgatggtggtgttttgagagaggttgtcaagcatgattccgttgaggaagcctggttatgtgacattgatgaagctgttattagactatcaaaggagtacctaccagaaatggctgcctcttattctcacccaaaggttaagacccacattggtgatggtttccaatttttaagagattaccaaaacacatttgacgtaatcattactgactcttctgacccagaaggtccagctgaaacccttttccaaaaggaatatttccaattgttgaacagtgcgttgacagaaaagggtgtaatcactacacaagcagaaagtatgtggattcacttgccaatcattaaggacttaaagaaagcctgttctgaagttttcccagttgcagaatactctttcgttactattccaacttacccaactggtacgattggttttatggtttgctccaaagataaaacttgcaatgtcaagaagccactacgtgaaatctctgatgagaaggaggctgaattatacagatactataacaagaaaattcacgaagcttcctttgttctaccaacctgggcagccaaggaattaaattag

SEQ ID NO: 34 - protein, 627 aa, Trypanosoma brucei
MTKSALADTKEEPHVPFGEIQGYTPCGVPAYSNGHDGFFSGERSIDGNLFCGFKYQCVEFARRWLYEAKGLVLPDVNWAAHIFDLTEVHDASTATPVPCVKVSNGTAAKPVADSLLIYAVNEDAPWGHVAVITEVGDKWVRIADQNHRFHKWKGTYSAELLLKHEGGVWTVEDHAAEGIFVPLGWVTFPSRPNRNPKEPLVLHESLYFKQPEKPFLRRVVFTPENRKTDWLDLTNEAEAEFYKTFGKEATRGGVYESCYYLMNRELYLNCVRYGTQLHSFFLEATKQVLESDDKLRRFRIPEEYWPRIRHSWKTQPHAITGRFDFVFDENTQEFKCFEYNADSASTLLECAVIQEKWANSVGLDDNATRSSGKFMPQTLVRAWEMTGLKGRVHFLVDDDGEERYTALYVMEKAREAGIDAKLCVMFDEFHFDEKGAVVDSDGIPATAVWKTWMWETAISDHQAAREQRGAEWKPTPKDKVRLCDILLGNNWDVRVFEPMWKLIPSNKAILPIIYNNHPDHPAILPASYELTDELRRTGYAKKPIVGRVGRNVTVTEPDGKVLAESDGNFSNRDMVYQQLFRIPKRGDYYAILGGWMLGDTYSGTGVREDKKLTTGLESPFGPVRIQM

SEQ ID NO: 35 - DNA, 1884 bp, Trypanosoma brucei
atgactaaatcagcattagcagatacaaaagaagaacctcacgtcccattcggtgaaatacaaggttataccccatgtggtgtcccagcttattcaaatggtcatgatggtttcttttctggtgaaagatcaatcgatggtaatttgttttgtggttttaaataccaatgtgttgaatttgctagaagatggttgtacgaagcaaaaggtttagttttgccagatgttaattgggctgcacatatcttcgatttgactgaagttcatgatgcttctactgcaacaccagttccatgtgttaaagtttcaaatggtacagctgcaaaaccagttgctgattctttgttgatctatgctgttaacgaagatgcaccatggggtcatgttgctgttattactgaagttggtgacaaatgggttagaatcgcagatcaaaaccatagattccataagtggaagggtacttactcagcagaattgttattgaaacatgaaggtggtgtttggacagttgaagatcatgctgcagaaggtatttttgttccattaggttgggttacatttccatcaagaccaaacagaaacccaaaggaaccattggttttgcatgaatctttgtacttcaagcaaccagaaaagccatttttgagaagagttgtttttacaccagaaaacagaaagactgattggttggatttgacaaacgaagctgaagcagaattttacaagactttcggtaaagaagctacaagaggtggtgtttacgaatcttgttactacttgatgaacagagaattgtatttgaattgtgttagatacggtactcaattgcattctttctttttggaagctacaaagcaagttttggaatctgatgataagttgagaagatttcgtattccagaagaatattggccaagaattagacattcatggaaaactcaaccacatgcaattacaggtagattcgatttcgttttcgatgaaaacactcaagagtttaaatgttttgaatacaatgctgattctgcatcaacattgttggaatgtgctgttattcaagaaaaatgggcaaattctgttggtttagatgataatgctactagatcttctggtaaattcatgccacaaactttggttagagcatgggaaatgacaggtttaaaaggtagagttcatttcttggttgatgatgatggtgaagaaagatacacagctttgtacgttatggaaaaagctagagaagcaggtatcgatgcaaagttgtgtgttatgttcgatgaatttcatttcgatgaaaaaggtgctgttgttgattcagatggtattccagctactgcagtttggaaaacatggatgtgggaaactgctatttctgatcatcaagctgcaagagaacaaagaggtgcagaatggaagccaactccaaaggataaagttagattgtgtgatatcttgttgggtaacaactgggatgttagagttttcgaaccaatgtggaagttgatcccatcaaataaggctatcttgccaatcatctataacaaccatccagatcatccagctattttgccagcatcttacgaattgactgatgaattgagaagaacaggttacgctaagaaaccaattgttggtagagttggtagaaatgttactgttacagaaccagatggtaaagttttggcagaatctgatggtaacttctcaaacagagatatggtttaccaacaattgtttagaataccaaaacgtggtgactattacgctattttaggtggttggatgttgggtgacacttactctggtacaggtgttagagaagataagaagttgactacaggtttagaatcccctttcggtccagttagaatccaaatgtga

SEQ ID NO: 36 - DNA, 27 bp, Artificial Sequence
  Primer
gtatgaagtaatacagaaaaggaaaac

SEQ ID NO: 37 - DNA, 46 bp, Artificial Sequence
  Primer
gacctgcagcgtacgaagcttcagcaatctctggtggaacgtctag

SEQ ID NO: 38 - DNA, 24 bp, Artificial Sequence
  Primer
ctgaagcttcgtacgctgcaggtc

SEQ ID NO: 39 - DNA, 24 bp, Artificial Sequence
  Primer
ggccactagtggatctgatatcac

SEQ ID NO: 40 - DNA, 52 bp, Artificial Sequence
  Primer
gtgatatcagatccactagtggcccctgaaccaaaacttcaaattcgaatac

SEQ ID NO: 41 - DNA, 25 bp, Artificial Sequence
  Primer
gcattcaaactctaaaataacaaag

SEQ ID NO: 42 - DNA, 48 bp, Artificial Sequence
  Primer
gtgatatcagatccactagtggccatagcttcaaaatgtttctactcc

SEQ ID NO: 43 - DNA, 28 bp, Artificial Sequence
  Primer
tttgtaattaaaacttagattagattgc

SEQ ID NO: 44 - DNA, 54 bp, Artificial Sequence
  Primer
gcaatctaatctaagttttaattacaaaatgtctagtactcaagtaggaaatgc

SEQ ID NO: 45 - DNA, 51 bp, Artificial Sequence
  Primer
gtgctagtgtctcccgtcttctgttcaatcgagttcagagtctatgtatac

SEQ ID NO: 46 - DNA, 24 bp, Artificial Sequence
  Primer
acagaagacgggagacactagcac

SEQ ID NO: 47 - DNA, 26 bp, Artificial Sequence
  Primer
attttcaacatcgtattttccgaagc

SEQ ID NO: 48 - DNA, 54 bp, Artificial Sequence
  Primer
gcttcggaaaatacgatgttgaaaatcctgaaccaaaacttcaaattcgaatac

SEQ ID NO: 49 - DNA, 50 bp, Artificial Sequence
  Primer
gtgatatcagatccactagtggcctgccgtaaaccactaaatcggaaccc

SEQ ID NO: 50 - DNA, 26 bp, Artificial Sequence
  Primer
tagggcccacaagcttacgcgtcgac

SEQ ID NO: 51 - DNA, 51 bp, Artificial Sequence
  Primer
gtcgacgcgtaagcttgtgggccctactaatttaattccttggctgcccag

SEQ ID NO: 52 - DNA, 52 bp, Artificial Sequence
  Primer
ctactttttacaacaaatataacaaaatggcacaagaaatcactcacccaac

SEQ ID NO: 53 - DNA, 26 bp, Artificial Sequence
  Primer
tttgttatatttgttgtaaaaagtag

SEQ ID NO: 54 - DNA, 26 bp, Artificial Sequence
  Primer
acgcacagatattataacatctgcac

SEQ ID NO: 55 - DNA, 50 bp, Artificial Sequence
  Primer
gtgcagatgttataatatctgtgcgtatagcttcaaaatgtttctactcc

SEQ ID NO: 56 - DNA, 51 bp, Artificial Sequence
  Primer
cttcggaaaatacgatgttgaaaatgttactccgcaacgcttttctgaacg

SEQ ID NO: 57 - DNA, 28 bp, Artificial Sequence
  Primer
taaagtaagagcgctacattggtctacc

SEQ ID NO: 58 - DNA, 56 bp, Artificial Sequence
  Primer
ggtagaccaatgtagcgctcttactttatcatattttcttctgcaatttcatatag

SEQ ID NO: 59 - DNA, 56 bp, Artificial Sequence
  Primer
gtttcgaataaacacacataaacaaacaaaatgactgtcaccataaaagaattgac

SEQ ID NO: 60 - DNA, 30 bp, Artificial Sequence
  Primer
tttgtttgtttatgtgtgtttattcgaaac

SEQ ID NO: 61 - DNA, 26 bp, Artificial Sequence
  Primer
tcgagtttatcattatcaatactgcc

SEQ ID NO: 62 - DNA, 54 bp, Artificial Sequence
  Primer
ggcagtattgataatgataaactcgacctgaaccaaaacttcaaattcgaatac

SEQ ID NO: 63 - DNA, 54 bp, Artificial Sequence
  Primer
ggcagtattgataatgataaactcgagtttaaagattacggatatttaacttac

SEQ ID NO: 64 - DNA, 34 bp, Artificial Sequence
  Primer
ttttagtttatgtatgtgttttttgtagttatag

SEQ ID NO: 65 - DNA, 61 bp, Artificial Sequence
  Primer
ctataactacaaaaaacacatacataaactaaaaatggttaataattcacagcatccttac

SEQ ID NO: 66 - DNA, 53 bp, Artificial Sequence
  Primer
gactaataattcttagttaaaagcacttcattcattaatgaccttgtctgccc

SEQ ID NO: 67 - DNA, 27 bp, Artificial Sequence
  Primer
agtgcttttaactaagaattattagtc

SEQ ID NO: 68 - DNA, 26 bp, Artificial Sequence
  Primer
aggtatcatctccatctcccatatgc

SEQ ID NO: 69 - DNA, 54 bp, Artificial Sequence
  Primer
gcatatgggagatggagatgatacctcctgaaccaaaacttcaaattcgaatac

SEQ ID NO: 70 - DNA, 45 bp, Artificial Sequence
  Primer
cgtcctctcgaaaggtggtttaaagattacggatatttaacttac

SEQ ID NO: 71 - DNA, 56 bp, Artificial Sequence
  Primer
ctacaaaaaacacatacataaactaaaaatgaacaggattaagaatacattttctg

SEQ ID NO: 72 - DNA, 53 bp, Artificial Sequence
  Primer
ggtcgacgcgtaagcttgtgggccctattaccaatagtttggaaataagtatc

SEQ ID NO: 73 - DNA, 38 bp, Artificial Sequence
  Primer
caggtggtcatggccctttgccgtaaaccactaaatcg

SEQ ID NO: 74 - DNA, 49 bp, Artificial Sequence
  Primer
ctacaaaaaacacatacataaactaaaaatgaccttggcacccctagac

SEQ ID NO: 75 - DNA, 49 bp, Artificial Sequence
  Primer
ggtcgacgcgtaagcttgtgggccctatcagttcaaatcagtaacaacc

SEQ ID NO: 76 - DNA, 60 bp, Artificial Sequence
  Primer
cattaaaaaactatatcaattaatttgaattaacttaccaatagtttggaaataagtatc

SEQ ID NO: 77 - DNA, 49 bp, Artificial Sequence
  Primer
catgactcgaggtcgacggtatctcagttcaaatcagtaacaacccttg

SEQ ID NO: 78 - DNA, 55 bp, Artificial Sequence
  Primer
gtaattatctactttttacaacaaatataacaaaatgaccttggcacccctagac

SEQ ID NO: 79 - DNA, 46 bp, Artificial Sequence
  Primer
caggtggtcatggccctttttgtaattaaaacttagattagattgc

SEQ ID NO: 80 - DNA, 58 bp, Artificial Sequence
  Primer
gcaatctaatctaagttttaattacaaaatgtccaagagcaaaactttcttatttacc

SEQ ID NO: 81 - DNA, 53 bp, Artificial Sequence
  Primer
ctaaatcattaaagtaacttaaggagttaaatttaaaattccaatttctttgg

SEQ ID NO: 82 - DNA, 32 bp, Artificial Sequence
  Primer
atttaactccttaagttactttaatgatttag

SEQ ID NO: 83 - DNA, 39 bp, Artificial Sequence
  Primer
caggtggtcatggcccttgcgaatttcttatgatttatg

SEQ ID NO: 84 - DNA, 21 bp, Artificial Sequence
  Primer
gcgaatttcttatgatttatg

SEQ ID NO: 85 - DNA, 48 bp, Artificial Sequence
  Primer
cataaatcataagaaattcgctcattttttcaacgcttccttttgagc

SEQ ID NO: 86 - DNA, 51 bp, Artificial Sequence
  Primer
cgaataaacacacataaacaaacaaaatgtctatagcaagttatgcccaag

SEQ ID NO: 87 - DNA, 47 bp, Artificial Sequence
  Primer
caggtggtcatggccctttttttgattaaaattaaaaaaactttttg

SEQ ID NO: 88 - DNA, 29 bp, Artificial Sequence
  Primer
tttttgattaaaattaaaaaaactttttg

SEQ ID NO: 89 - DNA, 58 bp, Artificial Sequence
  Primer
caaaaagtttttttaattttaatcaaaaaatgtcaatgagtaatattgttgtttttgg

SEQ ID NO: 90 - DNA, 51 bp, Artificial Sequence
  Primer
cgtcctctcgaaaggtggttaattcaaattaattgatatagttttttaatg

SEQ ID NO: 91 - DNA, 81 bp, Artificial Sequence
  Primer
aagggccatgaccacctgatgcaccaattaggtaggtctggctatgtctatacctctggcaattcgccctatagtgagtcg

SEQ ID NO: 92 - DNA, 87 bp, Artificial Sequence
  Primer
cacctttcgagaggacgatgcccgtgtctaaatgattcgaccagcctaagaatgttcaacgagctccagcttttgttccctttagtg

SEQ ID NO: 93 - DNA, 47 bp, Artificial Sequence
  Primer
gcatcgtcctctcgaaaggtgtcgagtttatcattatcaatactgcc

SEQ ID NO: 94 - DNA, 32 bp, Artificial Sequence; identical to SEQ ID NO: 82
  Primer

SEQ ID NO: 95 - DNA, 21 bp, Artificial Sequence
  Primer
gcgaatttcttatgatttatg

SEQ ID NO: 96 - DNA, 37 bp, Artificial Sequence
  Primer
gattaatataattatataaaaatattatcttcttttc

SEQ ID NO: 97 - DNA, 23 bp, Artificial Sequence
  Primer
ctggtttgttttacaaccaaaag

SEQ ID NO: 98 - DNA, 47 bp, Artificial Sequence
  Primer
aactctttcataaaatggtatctttaactttttatttaatcgtaatg

SEQ ID NO: 99 - DNA, 19 bp, Artificial Sequence
  Primer
aatgacaagtttcatcatc

SEQ ID NO: 100 - DNA, 54 bp, Artificial Sequence
  Primer
gcaatctaatctaagttttaattacaaagttactccgcaacgcttttctgaacg

SEQ ID NO: 101 - DNA, 56 bp, Artificial Sequence
  Primer
gcaatctaatctaagttttaattacaaaatgaacaggattaagaatacattttctg

SEQ ID NO: 102 - DNA, 47 bp, Artificial Sequence
  Primer
ctagtgtctcccgtcttctgtttaccaatagtttggaaataagtatc

SEQ ID NO: 103 - DNA, 55 bp, Artificial Sequence
  Primer
ctataactacaaaaaacacatacataaactaaaaatgaccttggcacccctagac

SEQ ID NO: 104 - DNA, 49 bp, Artificial Sequence
  Primer
gactaataattcttagttaaaagcacttcagttcaaatcagtaacaacc

SEQ ID NO: 105 - DNA, 50 bp, Artificial Sequence
  Primer
gtcgacgcgtaagcttgtgggccctatcatgatgctgtaatagcagaatc

SEQ ID NO: 106 - DNA, 51 bp, Artificial Sequence
  Primer
ctactttttacaacaaatataacaaaatggaaggtggtggtgctagaaatg

SEQ ID NO: 107 - DNA, 55 bp, Artificial Sequence
  Primer
ctataactacaaaaaacacatacataaactaaaaatgccagagtatacgctactg

SEQ ID NO: 108 - DNA, 52 bp, Artificial Sequence
  Primer
gactaataattcttagttaaaagcacttcatatatcatatctacgatcatcg

SEQ ID NO: 109 - DNA, 22 bp, Artificial Sequence
  Primer
gtgaaggaacaactcgtgtctc

SEQ ID NO: 110 - DNA, 22 bp, Artificial Sequence
  Primer
gaggatacgtacatatgcaagc

SEQ ID NO: 111 - DNA, 25 bp, Artificial Sequence
  Primer
tgccgtaaaccactaaatcggaacc

SEQ ID NO: 112 - DNA, 70 bp, Artificial Sequence
  Primer
tcagacttcttaactcctgtaaaaacaaaaaaaaaaaaaggcatagcaataagctggagctcatagcttc

SEQ ID NO: 113 - DNA, 81 bp, Artificial Sequence
  Primer
gtgcctattgatgatctggcggaatgtctgccgtgccatagccatgccttcacatatagtccgcaaattaaagccttcgag

SEQ ID NO: 114 - DNA, 81 bp, Artificial Sequence
  Primer
actatatgtgaaggcatggctatggcacggcagacattccgccagatcatcaataggcaccttcgtacgctgcaggtcgac

SEQ ID NO: 115 - DNA, 120 bp, Artificial Sequence
  Primer
tgcgcatgtttcggcgttcgaaacttctccgcagtgaaagataaatgatcactcatcaaattccgtacatgttttagagctagaaatagcaagttaaaataaggctagtccgttatcaac

SEQ ID NO: 116 - DNA, 120 bp, Artificial Sequence
  Primer
gttgataacggactagccttattttaacttgctatttctagctctaaaacatgtacggaatttgatgagtgatcatttatctttcactgcggagaagtttcgaacgccgaaacatgcgca

SEQ ID NO: 117 - DNA, 120 bp, Artificial Sequence
  Primer
ataaaatatcaaaacgccgatgagacaggcaggataaagtgacagattcagttatacatttttattagcattgatattattattttaaaaagtctatttacttgtatatttatccgaata

SEQ ID NO: 118 - DNA, 120 bp, Artificial Sequence
  Primer
tattcggataaatatacaagtaaatagactttttaaaataataatatcaatgctaataaaaatgtataactgaatctgtcactttatcctgcctgtctcatcggcgttttgatattttat

SEQ ID NO: 119 - DNA, 20 bp, Artificial Sequence
  Primer
ttttgcaacatccgggcatg

SEQ ID NO: 120 - DNA, 20 bp, Artificial Sequence
  Primer
cggctagctggtatggatcg

SEQ ID NO: 121 - DNA, 24 bp, Artificial Sequence
  Primer
atagcttcaaaatgtttctactcc

SEQ ID NO: 122 - DNA, 20 bp, Artificial Sequence
  Primer
tgcgacagaagaaagggaag

SEQ ID NO: 123 - DNA, 21 bp, Artificial Sequence
  Primer
cgtctatgaggagactgttag

SEQ ID NO: 124 - DNA, 43 bp, Artificial Sequence
  Primer
ggagtagaaacattttgaagctatacgtgaccacttcgagagc

SEQ ID NO: 125 - DNA, 52 bp, Artificial Sequence
  Primer
gcaatctaatctaagttttaattacaaaatgactgtcaccataaaagaattg

SEQ ID NO: 126 - DNA, 52 bp, Artificial Sequence
  Primer
gtgctagtgtctcccgtcttctgttcatattttcttctgcaatttcatatag

SEQ ID NO: 127 - DNA, 45 bp, Artificial Sequence
  Primer
gcttcggaaaatacgatgttgaaaatcctgcataatcggcctcac

SEQ ID NO: 128 - DNA, 20 bp, Artificial Sequence
  Primer
ctcgccaaggcattaccatc

SEQ ID NO: 129 - DNA, 19 bp, Artificial Sequence
  Primer
gagaacgagaggacccaac

SEQ ID NO: 130 - DNA, 44 bp, Artificial Sequence
  Primer
ggttccgatttagtggtttacggcaacgtgaccacttcgagagc

SEQ ID NO: 131 - DNA, 54 bp, Artificial Sequence
  Primer
gtcgacgcgtaagcttgtgggccctatcatattttcttctgcaatttcatatag

SEQ ID NO: 132 - DNA, 50 bp, Artificial Sequence
  Primer
ctactttttacaacaaatataacaaaatgactgtcaccataaaagaattg

SEQ ID NO: 133 - DNA, 52 bp, Artificial Sequence
  Primer
ggtagaccaatgtagcgctcttactttatcattttttcaacgcttccttttg

SEQ ID NO: 134 - DNA, 55 bp, Artificial Sequence
  Primer
gtttcgaataaacacacataaacaaacaaaatgtctatagcaagttatgcccaag

SEQ ID NO: 135 - DNA, 45 bp, Artificial Sequence
  Primer
ggcagtattgataatgataaactcgacctgcataatcggcctcac

SEQ ID NO: 136 - DNA, 49 bp, Artificial Sequence
  Primer
gactaataattcttagttaaaagcactctaccatccatgctcgaacaac

SEQ ID NO: 137 - DNA, 49 bp, Artificial Sequence; identical to SEQ ID NO: 136
  Primer

SEQ ID NO: 138 - DNA, 45 bp, Artificial Sequence
  Primer
gcatatgggagatggagatgatacctcctgcataatcggcctcac

SEQ ID NO: 139 - DNA, 36 bp, Artificial Sequence
  Primer
gggtaccggccgcaaattaaagccttcgagcgtccc

SEQ ID NO: 140 - DNA, 22 bp, Artificial Sequence
  Primer
gtgttcattgtacgtcctagac

SEQ ID NO: 141 - DNA, 19 bp, Artificial Sequence
  Primer
gtgcccaaagctaagagtc

SEQ ID NO: 142 - DNA, 18 bp, Artificial Sequence
  Primer
ctgctcttgaatggcgac

SEQ ID NO: 143 - DNA, 38 bp, Artificial Sequence
  Primer
gtcgccattcaagagcagcatcgtcctctcgaaaggtg

SEQ ID NO: 144 - DNA, 36 bp, Artificial Sequence
  Primer
cgaatcttcccatgcctgcaggtggtcatggccctt

SEQ ID NO: 145 - DNA, 18 bp, Artificial Sequence
  Primer
caggcatgggaagattcg

SEQ ID NO: 146 - DNA, 21 bp, Artificial Sequence
  Primer
ctggtgaggatttacggtatg

SEQ ID NO: 147 - DNA, 20 bp, Artificial Sequence
  Primer
gtgcgttatcgggttcttac

SEQ ID NO: 148 - DNA, 22 bp, Artificial Sequence
  Primer
caggttagttacttgctctatg

SEQ ID NO: 149 - DNA, 41 bp, Artificial Sequence
  Primer
cagtattgataatgataaactcgaaatcagacgcacgcttg

SEQ ID NO: 150 - DNA, 40 bp, Artificial Sequence
  Primer
ctttaatttgcggccggtacccttacgtggattgagccag

SEQ ID NO: 151 - DNA, 24 bp, Artificial Sequence
  Primer
gattgtcataataggagctatttg

SEQ ID NO: 152 - DNA, 25 bp, Artificial Sequence
  Primer
ccatagtattactattggtgttcat

SEQ ID NO: 153 - DNA, 22 bp, Artificial Sequence
  Primer
gttatcggttgtgatattgttc

SEQ ID NO: 154 - DNA, 22 bp, Artificial Sequence
  Primer
ttaagctattgtttcggcaatt

SEQ ID NO: 155 - DNA, 29 bp, Artificial Sequence
  Primer
cgtgcgauctctataaaaaatgtgcgaac

SEQ ID NO: 156 - DNA, 27 bp, Artificial Sequence
  Primer
atgacagautggtgttgtggttctgtg

SEQ ID NO: 157 - DNA, 21 bp, Artificial Sequence
  Primer
gagatctttgtgttcggttac

SEQ ID NO: 158 - DNA, 18 bp, Artificial Sequence
  Primer
agtctcgtatgtcggctc

SEQ ID NO: 159 - DNA, 18 bp, Artificial Sequence
  Primer
tgtgtccgcgtttctaag

SEQ ID NO: 160 - DNA, 21 bp, Artificial Sequence
  Primer
gaggtggttattgatcaccag

SEQ ID NO: 161 - DNA, 18 bp, Artificial Sequence
  Primer
acgaatcgttaggcacag

SEQ ID NO: 162 - DNA, 17 bp, Artificial Sequence
  Primer
gtgcaataccaaaatcg

SEQ ID NO: 163 - DNA, 28 bp, Artificial Sequence
  Primer
gcagttgtttggattaaaaagctgtacg

SEQ ID NO: 164 - DNA, 25 bp, Artificial Sequence
  Primer
ccttgtgtcatcatttactccaggc

SEQ ID NO: 165 - DNA, 26 bp, Artificial Sequence
  Primer
gtagagtcttagctgcagttggtatg

SEQ ID NO: 166 - DNA, 24 bp, Artificial Sequence
  Primer
cagggcattattactgacggcatg

SEQ ID NO: 167 - DNA, 26 bp, Artificial Sequence
  Primer
ctacagcacctttgaaagaaggtgtc

SEQ ID NO: 168 - DNA, 23 bp, Artificial Sequence
  Primer
gttgatggtgtcgtagacgtcag

SEQ ID NO: 169 - DNA, 20 bp, Artificial Sequence
  Primer
ggaacgtggatttaccccag

SEQ ID NO: 170 - DNA, 26 bp, Artificial Sequence
  Primer
gttatcggttgtgatattgttcctgc

SEQ ID NO: 171 - DNA, 20 bp, Artificial Sequence
  Primer
caaagcgatgggctccagac

SEQ ID NO: 172 - DNA, 23 bp, Artificial Sequence
  Primer
cattccgcagttaacatgtggtc

SEQ ID NO: 173 - DNA, 28 bp, Artificial Sequence
  Primer
gtgttcattgtacgtcctagactcaaac

SEQ ID NO: 174 - DNA, 21 bp, Artificial Sequence
  Primer
cgtgcgttatcgggttcttac

SEQ ID NO: 175 - DNA, 59 bp, Artificial Sequence
  Primer
ggaacactggggcaataggctgtcgccattcaagagcagcatcgtcctctcgaaaggtg

SEQ ID NO: 176 - DNA, 60 bp, Artificial Sequence
  Primer
ctattgtaattcaaaaaaaaaaagcgaatcttcccatgcctgcaggtggtcatggccctt

SEQ ID NO: 177 - DNA, 24 bp, Artificial Sequence
  Primer
cttgcataaattggtcaatgcaag

SEQ ID NO: 178 - DNA, 25 bp, Artificial Sequence
  Primer
cgatgacctcccattgatatttaag

SEQ ID NO: 179 - DNA, 24 bp, Artificial Sequence
  Primer
catcgtcaatttgtgatcgaagac

SEQ ID NO: 180 - DNA, 19 bp, Artificial Sequence
  Primer
cattcgccaggtagcttac

SEQ ID NO: 181 - DNA, 21 bp, Artificial Sequence
  Primer
tgcattttgagcgttgaacaa

SEQ ID NO: 182 - DNA, 20 bp, Artificial Sequence
  Primer
gtgccctgttctctgtagtt

SEQ ID NO: 183 - DNA, 60 bp, Artificial Sequence
  Primer
atcgggccctccttactgctctccttccgtgtaacgcgtttgccgtaaaccactaaatcg

SEQ ID NO: 184 - DNA, 57 bp, Artificial Sequence
  Primer
cgttaagaaaaatttcgagagagtcgccgatagtagattttcaacatcgtattttcc

SEQ ID NO: 185 - DNA, 57 bp, Artificial Sequence
  Primer
cctccttactgctctccttccgtgtaacgcgttatagcttcaaaatgtttctactcc

SEQ ID NO: 186 - DNA, 30 bp, Artificial Sequence
  Primer
ttatcgagctaactattttcgacacacatg

SEQ ID NO: 187 - DNA, 22 bp, Artificial Sequence
  Primer
cgtcgcccagtaagtgagacta

SEQ ID NO: 188 - DNA, 59 bp, Artificial Sequence
  Primer
gaaagcatagcaatctaatctaagttttaattacaaaatgtcaatgagtaatattgttg

SEQ ID NO: 189 - DNA, 51 bp, Artificial Sequence
  Primer
aagttgtgtgctagtgtctcccgtcttctgtctaccatccatgctcgaaca

SEQ ID NO: 190 - DNA, 60 bp, Artificial Sequence
  Primer
ctcgcctagtaaataaacgataaacaaatttgaagtagtagatacacgtatctcgacatg

SEQ ID NO: 191 - DNA, 59 bp, Artificial Sequence
  Primer
gaatgcaacaccgtagcatgaatcttgagattgcatctgataatgggttagtagtttat

SEQ ID NO: 192 - DNA, 70 bp, Artificial Sequence
  Primer
tctccgcagtgaaagataaatgatcaatttacgaaaaataaaggcgttttagagctagaaatagcaagtt

SEQ ID NO: 193 - DNA, 70 bp, Artificial Sequence
  Primer
aacttgctatttctagctctaaaacgcctttatttttcgtaaattgatcatttatctttcactgcggaga

SEQ ID NO: 194 - DNA, 60 bp, Artificial Sequence
  Primer
aataaaggcaaaaacagtggtcgtgtgagaaatctattttttcgaaattacttacacttt

SEQ ID NO: 195 - DNA, 60 bp, Artificial Sequence
  Primer
aaagtgtaagtaatttcgaaaaaatagatttctcacacgaccactgtttttgcctttatt

SEQ ID NO: 196 - DNA, 20 bp, Artificial Sequence
  Primer
ggtcacccacccatatacgg

SEQ ID NO: 197 - DNA, 20 bp, Artificial Sequence
  Primer
tgtcctccggataactgcac

SEQ ID NO: 198 - DNA, 120 bp, Artificial Sequence
  Primer
tgcgcatgtttcggcgttcgaaacttctccgcagtgaaagataaatgatcaatttacgaaaaataaaggcgttttagagctagaaatagcaagttaaaataaggctagtccgttatcaac

SEQ ID NO: 199 - DNA, 120 bp, Artificial Sequence
  Primer
gttgataacggactagccttattttaacttgctatttctagctctaaaacgcctttatttttcgtaaattgatcatttatctttcactgcggagaagtttcgaacgccgaaacatgcgca

SEQ ID NO: 200 - DNA, 120 bp, Artificial Sequence
  Primer
tctttttttgttcccaacaagaagtgagttaataaaggcaaaaacagtggtcgtgtgagaaatctattttttcgaaattacttacacttttgacggctagaaaaggatatacatacatat

SEQ ID NO: 201 - DNA, 120 bp, Artificial Sequence
  Primer
atatgtatgtatatccttttctagccgtcaaaagtgtaagtaatttcgaaaaaatagatttctcacacgaccactgtttttgcctttattaactcacttcttgttgggaacaaaaaaaga

SEQ ID NO: 202 - DNA, 120 bp, Artificial Sequence
  Primer
tgcgcatgtttcggcgttcgaaacttctccgcagtgaaagataaatgatcatcttcaaatccactacatagttttagagctagaaatagcaagttaaaataaggctagtccgttatcaac

SEQ ID NO: 203 - DNA, 120 bp, Artificial Sequence
  Primer
gttgataacggactagccttattttaacttgctatttctagctctaaaactatgtagtggatttgaagatgatcatttatctttcactgcggagaagtttcgaacgccgaaacatgcgca

SEQ ID NO: 204 - DNA, 120 bp, Artificial Sequence
  Primer
ttgtacgcttcacatagtagttcagtcaagaagagcaaacactaataagcaataaatctaggagaatatacatatatatgcatatgtttgtttagctaaataattttattgagctttgct

SEQ ID NO: 205 - DNA, 120 bp, Artificial Sequence
  Primer
agcaaagctcaataaaattatttagctaaacaaacatatgcatatatatgtatattctcctagatttattgcttattagtgtttgctcttcttgactgaactactatgtgaagcgtacaa

SEQ ID NO: 206 - DNA, 21 bp, Artificial Sequence
  Primer
acaccaatattctgcacctgc

SEQ ID NO: 207 - DNA, 20 bp, Artificial Sequence
  Primer
tgctggagaagatcgtacgc

SEQ ID NO: 208 - DNA, 120 bp, Artificial Sequence
  Primer
tgcgcatgtttcggcgttcgaaacttctccgcagtgaaagataaatgatcttagtagtttttggaaggatgttttagagctagaaatagcaagttaaaataaggctagtccgttatcaac

SEQ ID NO: 209 - DNA, 120 bp, Artificial Sequence
  Primer
gttgataacggactagccttattttaacttgctatttctagctctaaaacatccttccaaaaactactaagatcatttatctttcactgcggagaagtttcgaacgccgaaacatgcgca

SEQ ID NO: 210 - DNA, 120 bp, Artificial Sequence
  Primer
ttcttttttatattttttaggttttcatatagtgtcttacgcaaataggcggaccatagaaaagccgccatttgtgtctcctcatacttacatagaatagccctcttctattatccttcg

SEQ ID NO: 211 - DNA, 120 bp, Artificial Sequence
  Primer
cgaaggataatagaagagggctattctatgtaagtatgaggagacacaaatggcggcttttctatggtccgcctatttgcgtaagacactatatgaaaacctaaaaaatataaaaaagaa

SEQ ID NO: 212 - DNA, 20 bp, Artificial Sequence
  Primer
tacagctcgctccttgcatc

SEQ ID NO: 213 - DNA, 20 bp, Artificial Sequence
  Primer
gcttgcttggagggcttttc

SEQ ID NO: 214 - DNA, 120 bp, Artificial Sequence
  Primer
tgcgcatgtttcggcgttcgaaacttctccgcagtgaaagataaatgatcaagaaccctttatcataattgttttagagctagaaatagcaagttaaaataaggctagtccgttatcaac

SEQ ID NO: 215 - DNA, 120 bp, Artificial Sequence
  Primer
gttgataacggactagccttattttaacttgctatttctagctctaaaacaattatgataaagggttcttgatcatttatctttcactgcggagaagtttcgaacgccgaaacatgcgca

SEQ ID NO: 216 - DNA, 120 bp, Artificial Sequence
  Primer
ttttttgattgttctacaactttttcatagtaatcaaaacctttgaatttcaaacttactaggatatatttaaccacgactttcgcaagagagacggagggggtgggaaaaggctgaatg

SEQ ID NO: 217 - DNA, 120 bp, Artificial Sequence
  Primer
cattcagccttttcccaccccctccgtctctcttgcgaaagtcgtggttaaatatatcctagtaagtttgaaattcaaaggttttgattactatgaaaaagttgtagaacaatcaaaaaa

SEQ ID NO: 218 - DNA, 20 bp, Artificial Sequence
  Primer
tcatccaggtttcagcacgg

SEQ ID NO: 219 - DNA, 20 bp, Artificial Sequence
  Primer
agctcgaacaaggtgtcagg

SEQ ID NO: 220 - DNA, 60 bp, Artificial Sequence
  Primer
gattacttaccaatgtgccataaactccgtgcaccaatagcttcaaaatgtttctactcc

SEQ ID NO: 221 - DNA, 60 bp, Artificial Sequence
  Primer
cttgggttgtgggcaattgggtgtactatgaagcattttcaacatcgtattttccgaagc

SEQ ID NO: 222 - DNA, 47 bp, Artificial Sequence
  Primer
gcaatctaatctaagttttaattacaaaatggcacaagaaatcactc

SEQ ID NO: 223 - DNA, 45 bp, Artificial Sequence
  Primer
gtgctagtgtctcccgtcttctgtctaatttaattccttggctgc

SEQ ID NO: 224 - DNA, 25 bp, Artificial Sequence
  Primer
gaccatcactaaagcttctctctta

SEQ ID NO: 225 - DNA, 25 bp, Artificial Sequence
  Primer
ttgagcaattcatcgacaacaagag

SEQ ID NO: 226 - DNA, 43 bp, Artificial Sequence
  Primer
agaaccagaaccagatcctattttcttctgcaatttcatatag

SEQ ID NO: 227 - DNA, 37 bp, Artificial Sequence
  Primer
ggatctggttctggttctatggcacaagaaatcactc

SEQ ID NO: 228 - DNA, 36 bp, Artificial Sequence
  Primer
agaaccagaaccagatccatttaattccttggctgc

SEQ ID NO: 229 - DNA, 42 bp, Artificial Sequence
  Primer
ggatctggttctggttctatgactgtcaccataaaagaattg

SEQ ID NO: 230 - DNA, 60 bp, Artificial Sequence
  Primer
gattacttaccaatgtgccataaactccgtgcaccatgccgtaaaccactaaatcggaac

SEQ ID NO: 231 - DNA, 120 bp, Artificial Sequence
  Primer
tgcgcatgtttcggcgttcgaaacttctccgcagtgaaagataaatgatcttcttagattactattatatgttttagagctagaaatagcaagttaaaataaggctagtccgttatcaac

SEQ ID NO: 232 - DNA, 120 bp, Artificial Sequence
  Primer
gttgataacggactagccttattttaacttgctatttctagctctaaaacatataatagtaatctaagaagatcatttatctttcactgcggagaagtttcgaacgccgaaacatgcgca

SEQ ID NO: 233 - DNA, 120 bp, Artificial Sequence
  Primer
atagttattttgaaataataactaccattagaactaacaaaagaaaagaaaaaaaaaatataccatttgcaagacattgtataatatttttgttgaaagtctttttcgattcataagcgc

SEQ ID NO: 234 - DNA, 120 bp, Artificial Sequence
  Primer
gcgcttatgaatcgaaaaagactttcaacaaaaatattatacaatgtcttgcaaatggtatattttttttttcttttcttttgttagttctaatggtagttattatttcaaaataactat

SEQ ID NO: 235 - DNA, 20 bp, Artificial Sequence
  Primer
ctcatcgcatgccaacgaag

SEQ ID NO: 236 - DNA, 20 bp, Artificial Sequence
  Primer
gcagcaaagccaacccttac

SEQ ID NO: 237 - DNA, 49 bp, Artificial Sequence
  Primer
gcaatctaatctaagttttaattacaaaatgtctgttcactctatcttg

SEQ ID NO: 238 - DNA, 45 bp, Artificial Sequence
  Primer
gtgctagtgtctcccgtcttctgtttattcaaccatcttctttgg

SEQ ID NO: 239 - DNA, 45 bp, Artificial Sequence
  Primer
caatgtagcgctcttactttattatttcaagtccttgaaattacc

SEQ ID NO: 240 - DNA, 22 bp, Artificial Sequence
  Primer
ctggagctcagtttatcattat

SEQ ID NO: 241 - DNA, 21 bp, Artificial Sequence
  Primer
actatagggcgaattgggtac

SEQ ID NO: 242 - DNA, 40 bp, Artificial Sequence
  Primer
ataatgataaactgagctccagagtctcgtatgtcggctc

SEQ ID NO: 243 - DNA, 39 bp, Artificial Sequence
  Primer
gtacccaattcgccctatagttgtgtccgcgtttctaag

SEQ ID NO: 244 - DNA, 52 bp, Artificial Sequence
  Primer
ggtagaccaatgtagcgctcttactttattattcaaccatcttctttggaac

SEQ ID NO: 245 - DNA, 52 bp, Artificial Sequence
  Primer
gtttcgaataaacacacataaacaaacaaaatgtctgttcactctatcttgt

SEQ ID NO: 246 - DNA, 57 bp, Artificial Sequence
  Primer
ctataactacaaaaaacacatacataaactaaaaatgccgtttggaatagacaacac

SEQ ID NO: 247 - DNA, 50 bp, Artificial Sequence
  Primer
gactaataattcttagttaaaagcactttaccagacatcttcttggtatc

SEQ ID NO: 248 - DNA, 19 bp, Artificial Sequence
  Primer
tggtcacacaacttgtctg

SEQ ID NO: 249 - DNA, 20 bp, Artificial Sequence
  Primer
ggtactggtggtttcacttg

SEQ ID NO: 250 - DNA, 21 bp, Artificial Sequence
  Primer
gtagtgatcattggcttaacg

SEQ ID NO: 251 - DNA, 46 bp, Artificial Sequence
  Primer
gttccgatttagtggtttacggcagtgacaataaattcaaaccggt

SEQ ID NO: 252 - DNA, 46 bp, Artificial Sequence
  Primer
gcttcggaaaatacgatgttgaaaatcaactcagaagtttgacagc

SEQ ID NO: 253 - DNA, 21 bp, Artificial Sequence
  Primer
tcgttagattctgtatcccta

SEQ ID NO: 254 - DNA, 46 bp, Artificial Sequence
  Primer
ggagtagaaacattttgaagctatgtgacaataaattcaaaccggt

SEQ ID NO: 255 - DNA, 22 bp, Artificial Sequence
  Primer
atttgtgatcgaagacgaagag

SEQ ID NO: 256 - DNA, 18 bp, Artificial Sequence
  Primer
tcaagaagccactacgtg

SEQ ID NO: 257 - DNA, 24 bp, Artificial Sequence
  Primer
actagaacattaccatatgtagtg

SEQ ID NO: 258 - DNA, 20 bp, Artificial Sequence
  Primer
caacttggacgttcttctac

SEQ ID NO: 259 - protein, 619 aa, Escherichia coli
MSKGTTSLDAPFGTLLGYAPGGVAIYSSDYSSLDPQEYEDDAVFRSYIDDEYMGHKWQCVEFARRFLFLNYGVVFTDVGMAWEIFSLRFLREVVNDNILPLQAFPNGSPRAPVAGALLIWDKGGEFKDTGHVAIITQLHGNKVRIAEQNVIHTPLPQGQQWTRELEMVVENGGYTLKDTFDDTTILGWMIQTEDTEYSLPQPEIAGELLKISGARLENKGQFDGKWLDEKDPLQNAYVQANGQVINQDPYHYYTITESAEQELIKATNELHLMYLHATDKVLKDDNLLALFDIPKILWPRLRLSWQRRRHHMITGRMDFCMDERGLKVYEYNADSASCHTEAGLILERWAEQGYKGNGFNPAEGLINELAGAWKHSRARPFVHIMQDKDIEENYHAQFMEQALQQAGFETRILRGLDELGWDAAGQLIDGEGRLVNCVWKTWAWETAFDQIREVSDREFAAVPIRTGHPQNEVRLIDVLLRPEVLVFEPLWTVIPGNKAILPILWSLFPHHRYLLDTDFTVNDELVKTGYAVKPIAGRCGSNIDLVSHHEEVLDQTSGKFAEQKNIYQQLWCLPKVDGKYIQVCTFTVGGNYGGTCLRGDESLVIKKESDIEPLIVVKE

SEQ ID NO: 260 - DNA, 1860 bp, Escherichia coli
atgagtaaagggaccaccagtttggacgccccttttggaacattgttaggatacgcccctgggggggtagcaatttatagcagtgactattcctcactggacccacaagaatatgaagatgatgccgtcttccgtagttatatagatgatgaatacatgggccataaatggcaatgcgttgagtttgctaggcgtttcttattcttgaactatggtgtcgtatttactgacgtgggaatggcttgggagatattctctctaagatttctgcgtgaggtcgtcaatgataacatcctgccgttacaagcatttccaaatggttcacccagagcccctgtagctggtgctctactaatatgggataaaggaggtgaatttaaggatactgggcacgtagcgataattacgcaattacacggcaacaaagtaagaattgcagaacaaaatgtcatccatacaccactacctcaaggacaacagtggactagagagctagaaatggttgtggaaaacggtgggtatacgttaaaagatacgttcgacgataccaccattcttggttggatgatccagacggaagacactgagtactctctgccacagcctgagattgccggcgaattattgaaaatcagcggagcacgtttggaaaacaaagggcaattcgacgggaagtggctggatgagaaagatcctttacagaacgcctacgtacaagccaacgggcaggttatcaaccaagacccgtatcattattacacgattacagaaagtgccgagcaggagctaatcaaggccacaaacgagttacaccttatgtacttgcatgccacggacaaggtcttgaaagacgacaatcttttagccctatttgacattcctaagatcctatggcctaggctacgtttatcctggcagcgtaggcgtcaccacatgatcacgggacgtatggacttctgcatggatgaacgtgggctgaaggtttatgaatacaacgccgactctgccagttgccatacagaagctggccttatcctggagagatgggctgagcaggggtacaaaggaaacggattcaacccagccgaaggattaatcaacgaactggccggtgcatggaagcatagcagggctcgtccattcgtccatatcatgcaggacaaggatatcgaggaaaattaccatgcgcagtttatggaacaagcactacaacaagctgggtttgagacccgtatactaaggggccttgatgagcttggctgggacgctgctggccagttgattgatggagaaggacgtcttgtgaattgcgtttggaaaacgtgggcatgggagacagccttcgatcagataagagaagtatcagatagagaatttgctgctgtaccgattagaacaggccacccgcaaaacgaagtaagactgatagacgtattgttaagacctgaggtgctggtttttgaacctctttggacagtaatacctggaaacaaagcaatacttccgatcttatggtcactattcccgcaccacaggtatttgttggacaccgattttactgtcaacgatgaactggtcaagacggggtacgcggtcaaacccatagctggccgttgtggcagtaatatagatttggtatctcatcacgaggaggtcttggatcaaactagtgggaaatttgccgaacagaaaaatatttatcagcagctgtggtgcttgcctaaagttgatgggaaatatattcaagtctgtacttttacagtgggaggtaattacggaggcacgtgtttgagaggtgacgaatcacttgtaataaagaaagagtctgatattgagcccttaattgtagtgaaggaatag

(19) United States
(12) Patent Application Publication
(10) Pub. No.: US 2024/0294927 A1
(43) Pub. Date: Sep. 5, 2024

(54) POLYAMINE CONJUGATE PRODUCING YEASTS

(71) Applicant: CHRYSEA LIMITED, Dublin (IE)

(72) Inventors: Jiufu Qin, Kongens Lyngby (DK); Jens Nielsen, Hellerup (DK)

(21) Appl. No.: 17/768,618

(22) PCT Filed: Oct. 27, 2020
(86) PCT No.: PCT/EP2020/080140

§ 371 Date: Apr. 13, 2022

(30) Foreign Application Priority Data
Oct. 28, 2019 (SE) 1951232-6

(51) Int. Cl. C12N 15/52; C12N 1/16; C12N 9/00; C12N 9/10; C12P 21/02

(52) U.S. Cl. CPC C12N 15/52; C12N 1/165; C12N 9/1085; C12N 9/93; C12P 21/02; C12Y 205/01022; C12Y 205/01044; C12Y 205/01079; C12Y 603/01008; C12Y 603/01009

(57) ABSTRACT

The invention relates to production of polyamine conjugates in yeast cells that are capable of producing at least one polyamine. The yeast cells also comprise a polyamine:glutathione ligase encoding gene and at least one polyamine synthase encoding gene but lacks a polyamine oxidase encoding gene or comprises a disrupted polyamine oxidase encoding gene. The yeast cells are capable of producing various polyamine-glutathione conjugates.

### TECHNICAL FIELD

The present invention generally relates to genetically engineered yeasts, and in particular to such yeasts capable of producing polyamine conjugates.

### BACKGROUND

Small molecules with novel mechanisms of action are needed to help address today's most challenging biomedical and agriculture problems. During decades, however, more than 135 million of small molecules generated by synthetic chemistry aiming to identify lead compounds for pharmaceutical and agrochemical development, phased out in spite of passing the initial screening. It has become clear that improving the complexity and diversity of the small molecules is necessary. Actually, diverse compounds, namely nature products and their derivatives with complex structures are readily available in nature, which have played important roles in the study and treatment of disease for thousands of years. In particular, polyamine conjugates, a diverse and quantitatively major group of secondary metabolites of plants show extensive structural diversity and complexity. Thereby multiple studies have reported on the beneficial properties of these compounds. For instance, polyamine-glutathione conjugates, also known as trypanothione, have been proposed as specific molecular probes in medicinal chemistry and for the development of glutathione reductase inhibitors.

Unfortunately, due to the structural complexity and low abundance in nature, it is hard to obtain polyamine analogs from either traditional synthetic chemistry or extraction from the natural sources. Microbial-based production in fast-growing, genetically tractable species has been pursued as an alternative to the traditional supply chains for natural products and their derivatives. In particular, the baker's yeast Saccharomyces cerevisiae has served as a cell factory for producing many different fuels, chemicals, food ingredients, and pharmaceuticals, in particular for its production of natural products.

Consequently, it is desirable to develop new methods for the discovery, and production of polyamine conjugates.

### SUMMARY

It is a general objective to provide yeast cells capable of producing polyamine conjugates.

This and other objectives are met by the embodiments.

The present invention is defined in the independent claims. Further embodiments of the invention are defined in the dependent claims.

The invention relates to a yeast cell capable of producing glutathione-polyamine conjugates. The yeast cell is capable of producing at least one polyamine. The yeast cell also comprises a polyamine:glutathione ligase encoding gene and at least one polyamine synthase encoding gene but lacks a polyamine oxidase encoding gene or comprises a disrupted polyamine oxidase encoding gene.

The invention also relates to a method of producing glutathione-polyamine conjugates. The method comprises culturing a yeast cell according to the invention in a culture medium and in culture conditions suitable for production of the glutathione-polyamine conjugates by the yeast cell. The method also comprises collecting the glutathione-polyamine conjugates from the culture medium and/or from the yeast cell.

The present invention provides an efficient means for the production of various glutathione-polyamine conjugates, including mono- and/or multi-substituted polyamines, such as trypanothione. The invention can therefore be used as a cost efficient alternative to prior art methods involving traditional synthetic chemistry or extraction from natural sources to obtain polyamine conjugates.

### BRIEF DESCRIPTION OF THE DRAWINGS

The embodiments, together with further objects and advantages thereof, may best be understood by making reference to the following description taken together with the accompanying drawings, in which:

FIGS. 1a to 1f illustrate engineering yeast metabolism for spermidine and higher-polyamine over-synthesis.

FIGS. 2a and 2b illustrate trypanothione-(SH)₂ production in yeast.

FIGS. 3a and 3b illustrate the engineered pathways for the biosynthesis of complex phenolamides and trypanothione-(SH)₂ in yeast.

### DETAILED DESCRIPTION

To enable efficient access to the diversity of polyamine conjugates, we engineered yeast metabolism to over-produce a category of complex polyamines, e.g., spermidine, homo-spermidine, thermospermine, and spermine. The versatility of this yeast platform is demonstrated by biosynthesis of diverse polyamine conjugates with tailoring pathways. Specially, aided by computational simulations, we systematically refactored yeast central carbon and nitrogen metabolism, methionine salvage pathway, salvage pathways of adenine, polyamine transport machinery, and polyamine degradation pathway, thereby enabling yeasts capable of producing >400 mg/l spermidine in deep-well scale fermentation. Furthermore, by plugging in tailoring pathways and creating synthetic consortium, we demonstrated the de novo biosynthesis of polyamine conjugates including trypanothione in yeast.

The present invention now will be described hereinafter with reference to the accompanying drawings and examples, in which embodiments of the invention are shown. This description is not intended to be a detailed catalogue of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined herein, scientific and technical terms used herein will have the meanings that are commonly understood by those of ordinary skill in the art.

Generally, nomenclatures used in connection with techniques of biochemistry, enzymology, molecular and cellular biology, microbiology, genetics and protein and nucleic acid chemistry and hybridization, described herein, are those well-known and commonly used in the art.

Conventional methods and techniques mentioned herein are explained in more detail, for example, in Molecular Cloning, a laboratory manual [second edition] Sambrook et al. Cold Spring Harbor Laboratory, 1989, for example in Sections 1.21 “Extraction And Purification Of Plasmid DNA”, 1.53 “Strategies For Cloning In Plasmid Vectors”, 1.85 “Identification Of Bacterial Colonies That Contain Recombinant Plasmids”, 6 “Gel Electrophoresis Of DNA”, 14 “In vitro Amplification Of DNA By The Polymerase Chain Reaction”, and 17 “Expression Of Cloned Genes In Escherichia coli” thereof.

Enzyme Commission (EC) numbers (also called “classes” herein), referred to throughout this specification, are according to the Nomenclature Committee of the International Union of Biochemistry and Molecular Biology (NC-IUBMB) in its resource “Enzyme Nomenclature” (1992, including Supplements 6-17) available, for example, as “Enzyme nomenclature 1992: recommendations of the Nomenclature Committee of the International Union of Biochemistry and Molecular Biology on the nomenclature and classification of enzymes”, Webb, E. C. (1992), San Diego: Published for the International Union of Biochemistry and Molecular Biology by Academic Press (ISBN 0-12-227164-5). This is a numerical classification scheme based on the chemical reactions catalyzed by each enzyme class.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

As used herein, the transitional phrase “consisting” essentially of means that the scope of a claim is to be interpreted to encompass the specified materials or steps recited in the claim and those that do not materially affect the basic and novel characteristic(s) of the claimed invention. Thus, the term “consisting” essentially of when used in a claim of this invention is not intended to be interpreted to be equivalent to “comprising.”

To facilitate understanding of the invention, a number of terms are defined below.

As used herein, the term “polyamine” refers to an organic compound having two or more primary amino groups. Examples for polyamines include putrescine (Put), spermidine (Spd), spermine (Spm), thermospermine (Tspm), sym-homospermidine (Hspd), 1,2-diaminopropane, cadaverine, agmatine, sym-norspermidine and norspermine.

As used herein, the terms “polyamine conjugate” or “glutathione-polyamine conjugate” refers to a conjugate between at least one glutathione (GSH) molecule and a polyamine. The glutathione-polyamine conjugate preferably comprises an amide bound formed between the carboxyl group of GSH and an amine group of the polyamine. Non-limiting, but illustrative, examples of glutathione-polyamine conjugates include trypanothione (N¹,N¹⁰-bis(glutathionyl) spermidine), N¹-glutathionyl spermidine, N¹⁰-glutathionyl spermidine, N¹,N¹⁰-bis(glutathionyl) spermine, N¹, N⁵,N¹⁰-tri(glutathionyl) spermine, N¹,N⁵,N¹⁰,N¹⁴-tetra(glutathionyl) spermine. Hence, the glutathione-polyamine conjugate could be a conjugate between one polyamine, such as spermidine or spermine, and one, two or more, such as three or four, glutathione molecules.

Also as used herein, the terms “nucleotide sequence”, “nucleic acid”, “nucleic acid molecule”, “oligonucleotide” and “polynucleotide” refer to RNA or DNA, including cDNA, a DNA fragment or portion, genomic DNA, synthetic DNA, plasmid DNA, RNA, and anti-sense RNA, any of which can be single stranded or double stranded, linear or branched, or a hybrid thereof. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. As used herein the term “recombinant” when used means that a particular nucleic acid (DNA or RNA) is the product of various combinations of cloning, restriction, and/or ligation steps resulting in a construct having a structural coding or non-coding sequence distinguishable from endogenous nucleic acids found in natural systems.

As used herein, the term “gene” refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions, e.g., introns, regulatory elements, promoters, enhancers, termination sequences and/or 5′ and 3′ untranslated regions. A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

A “disrupted gene” as defined herein involves any mutation or modification to a gene resulting in a partial or fully non-functional gene and gene product. Such a mutation or modification includes, but is not limited to, a missense mutation, a nonsense mutation, a deletion, a substitution, an insertion, addition of a targeting sequence and the like. Furthermore, a disruption of a gene can be achieved also, or alternatively, by mutation or modification of control elements controlling the transcription of the gene, such as mutation or modification in a promoter, terminator and/or enhancement elements. In such a case, such a mutation or modification results in partially or fully loss of transcription of the gene, i.e., a lower or reduced transcription as compared to native and non-modified control elements. As a result a reduced, if any, amount of the gene product will be available following transcription and translation. Furthermore, disruption of a gene could also entail adding or removing a localization signal from the gene, resulting in decreased presence of the gene product in its native subcellular compartment.

The objective of gene disruption is to reduce the available amount of the gene product, including fully preventing any production of the gene product, or to express a gene product that lacks or having lower enzymatic activity as compared to the native or wild type gene product.

As used herein the term “deletion” or “knock-out” refers to a gene that is inoperative or knocked out.

The term “attenuated activity” when related to an enzyme refers to a decrease in the activity of the enzyme in its native compartment compared to a control or wildtype state. Manipulations that result in attenuated activity of an enzyme include, but are not limited to, a missense mutation, a nonsense mutation, a deletion, a substitution, an insertion, addition of a targeting sequence, removal of a targeting sequence, or the like. A cell that contains modifications that result in attenuated enzyme activity will have a lower activity of the enzyme compared to a cell that does not contain such modifications. Attenuated activity of an enzyme may be achieved by encoding a nonfunctional gene product, e.g., a polypeptide having essentially no activity, e.g., less than about 10% or even 5% as compared to the activity of the wild type polypeptide.

A codon optimized version of a gene refers to an exogenous gene introduced into a cell and where the codons of the gene have been optimized with regard to the particular cell. Generally, not all tRNAs are expressed equally or at the same level across species. Codon optimization of a gene sequence thereby involves changing codons to match the most prevalent tRNAs, i.e., to change a codon recognized by a low prevalent tRNA with a synonymous codon recognized by a tRNA that is comparatively more prevalent in the given cell. This way the mRNA from the codon optimized gene will be more efficiently translated. The codon and the synonymous codon preferably encode the same amino acid.

As used herein, the terms “peptide”, “polypeptide”, and “protein” are used interchangeably to indicate a polymer of amino acid residues. The terms “peptide”, “polypeptide” and “protein” also includes modifications including, but not limited to, lipid attachment, glycosylation, glycosylation, sulfation, hydroxylation, γ-carboxylation of L-glutamic acid residues and ADP-ribosylation.

As used herein, the term “enzyme” is defined as a protein which catalyzes a chemical or a biochemical reaction in a cell. Usually, according to the present invention, the nucleotide sequence encoding an enzyme is operably linked to a nucleotide sequence (promoter) that causes sufficient expression of the corresponding gene in the cell to confer to the cell the ability to produce spermidine.

As used herein, the term “open reading frame (ORF)” refers to a region of RNA or DNA encoding a polypeptide, a peptide, or a protein.

As used herein, the term “genome” encompasses both the plasmids and chromosomes in a host cell. For instance, encoding nucleic acids of the present disclosure which are introduced into host cells can be portion of the genome whether they are chromosomally integrated or plasmids-localized.

As used herein, the term “promoter” refers to a nucleic acid sequence which has functions to control the transcription of one or more genes, which is located upstream with respect to the direction of transcription of the transcription initiation site of the gene. Suitable promoters in this context include both constitutive and inducible natural promoters as well as engineered promoters, which are well known to the person skilled in the art.

Suitable promoters for use in yeast cells include, but are not limited to, the promoters of PDC, GPD1, TEF1, PGK1 and TDH. Other suitable promoters include the promoters of GAL1, GAL2, GAL10, GAL7, CUP1, HIS3, CYC1, ADH1, PGL, GAPDH, ADC1, URA3, TRP1, LEU2, TPI, AOX1 and ENOI.

As used herein, the term “terminator” refers to a “transcription termination signal” if not otherwise noted. Terminators are sequences that hinder or stop transcription of a polymerase.

As used herein, “recombinant eukaryotic cells” according to the present disclose is defined as cells which contain additional copies or copy of an endogenous nucleic acid sequence or are transformed or genetically modified with polypeptide or a nucleotide sequence that does not naturally occur in the eukaryotic cells. The wildtype eukaryotic cells are defined as the parental cells of the recombinant eukaryotic cells, as used herein.

As used herein, the terms “increase”. “increased”, “increasing”, “enhance”, “enhanced”, “enhancing”, and “enhancement” (and grammatical variations thereof) indicate an elevation of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more, or any range therein, as compared to a control.

As used herein, the terms “reduce”, “reduced”, “reduction”, “diminish”, “suppress”, and “decrease” and similar terms mean a decrease of at least about, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more, or any range therein, as compared to a control.

A reduced expression of a gene as used herein involves a genetic modification that reduces the transcription of the gene, reduces the translation of the mRNA transcribed from the gene and/or reduces post-translational processing of the protein translated from the mRNA. Such genetic modification includes insertion(s), deletion(s), replacement(s) or mutation(s) applied to the control sequence, such as a promoter and enhancer, of the gene. For instance, the promoter of the gene could be replaced by a less active or inducible promoter to thereby result in a reduced transcription of the gene. Also a knock-out of the promoter would result in reduced, typically zero, expression of the gene.

As used herein, the term “portion” or “fragment” of a nucleotide sequence of the invention will be understood to mean a nucleotide sequence of reduced length relative to a reference nucleic acid or nucleotide sequence and comprising, consisting essentially of and/or consisting of a nucleotide sequence of contiguous nucleotides identical or almost identical, e.g., 80%, 81%, 82%, 83%, 84%, 85%, 86%, 87%, 88%, 89%, 90%, 91%, 92%, 93%, 94%, 95%, 96%, 98%, 99% identical, to the reference nucleic acid or nucleotide sequence. Such a nucleic acid fragment or portion according to the invention may be, where appropriate, included in a larger polynucleotide of which it is a constituent.

Different nucleic acids or proteins having homology are referred to herein as “homologues”. The term homologue includes homologous sequences from the same and other species and orthologous sequences from the same and other species. “Homology” refers to the level of similarity between two or more nucleic acid and/or amino acid sequences in terms of percent of positional identity, i.e., sequence similarity or identity. Homology also refers to the concept of similar functional properties among different nucleic acids or proteins. Thus, the compositions and methods of the invention further comprise homologues to the nucleotide sequences and polypeptide sequences of this invention. “Orthologous”, as used herein, refers to homologous nucleotide sequences and/or amino acid sequences in different species that arose from a common ancestral gene during speciation. A homologue of a nucleotide sequence of this invention has a substantial sequence identity, e.g., at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, and/or 100%, to said nucleotide sequence.

The term “overexpress”, or “overexpression” as used herein refers to higher levels of activity of a gene, e.g., transcription of the gene; higher levels of translation of mRNA into protein; and/or higher levels of production of a gene product, e.g., polypeptide, than would be in the cell in its native or control, e.g., not transformed with the particular heterologous or recombinant polypeptides being overexpressed, state. A typical example of an overexpressed gene is a gene under transcription control of another promoter as compared to the native promoter of the gene. Also, or alternatively, other changes in the control elements of a gene, such as enhancers, could be used to overexpress the particular gene. Furthermore, modifications that affect, i.e., increase, the translation of the mRNA transcribed from the gene could, alternatively or in addition, be used to achieve an overexpressed gene as used herein. These terms can also refer to an increase in the number of copies of a gene and/or an increase in the amount of mRNA and/or gene product in the cell. It is further possible to achieve overexpression by including genes from different species encoding the same or homologous gene product, such as enzyme. Overexpression can result in levels that are 25%, 50%, 75%, 100%, 200%, 300%, 400%, 500%, 750%, 1000%, 1500%, 2000% or higher in the cell, or any range therein, as compared to control levels.

As used herein, the terms “exogenous” or “heterologous” when used with respect to a nucleic acid (RNA or DNA), protein or gene refer to a nucleic acid, protein or gene which occurs non-naturally as part of the cell, organism, genome, RNA or DNA sequence, into which it is introduced, including non-naturally occurring multiple copies of a naturally occurring nucleotide sequence. Such an exogenous gene could be a gene from another species or strain, a modified, mutated or evolved version of a gene naturally occurring in the host cell or a chimeric version of a gene naturally occurring in the host cell or fusion genes. In these former cases, the modification, mutation or evolution causes a change in the nucleotide sequence of the gene to thereby obtain a modified, mutated or evolved gene with another nucleotide sequence as compared to the gene naturally occurring in the host cell. Evolved gene refers to genes encoding evolved genes and obtained by genetic modification, such as mutation or exposure to an evolutionary pressure, to derive a new gene with a different nucleotide sequence as compared to the wild type or native gene. A chimeric gene is formed through the combination of portions of one or more coding sequences to produce a new gene. These modifications are distinct from a fusion gene, which merges whole gene sequences into a single reading frame and often retain their original functions.

An “endogenous”, “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the organism.

As used herein, the term “modified”, when it is used with respect to an organism, refers to a host organism that has been modified to produce polyamine conjugates, as compared with an otherwise identical host organism that has not been so modified. In principle, such “modification” in accordance with the present disclosure may comprise any physiological, genetic, chemical, or other modification that appropriately alters production of polyamine conjugates in a host organism as compared with an otherwise identical organism which is not subject to the modification. In most of the embodiments, however, the modification will comprise a genetic modification. In certain embodiments, as described herein, the modification comprises introducing genes into a host cell. Genetic modifications which boost the activity of a polypeptide include, but are not limited to: introducing one or more copies of a gene encoding the polypeptide (which may distinguish from any gene already present in the host cell encoding a polypeptide having the same activity); altering a gene present in the cell to increase transcription or translation of the gene (e.g., altering, adding additional sequence to, replacement of one or more nucleotides, deleting sequence from, or swapping for example, regulatory, a promoter or other sequence); and altering the sequence (e.g., non-coding or coding) of a gene encoding the polypeptide to boost activity (e.g., by increasing enzyme activity, decrease feedback inhibition, targeting a specific subcellular location, boost mRNA stability, boost protein stability). Genetic modifications that reduce activity of a polypeptide include, but are not limited to: deleting a portion or all of a gene encoding the polypeptide; inserting a nucleic acid sequence which disrupts a gene encoding the polypeptide; changing a gene present in the cell to reduce transcription or translation of the gene or stability of the mRNA or polypeptide encoded by the gene (for example, by adding additional sequence to, altering, deleting sequence from, replacement of one or more nucleotides, or swapping for example, replacement of one or more nucleotides, a promoter, regulatory or other sequence). The term “overproducing” is used herein in reference to the production of a product in a host cell and indicates that the host cell is producing more of product by virtue of the introduction of nucleic acid sequences which encode different polypeptides involved in the host cell's metabolic pathways or as a result of other modifications as compared with the unmodified host cell or wild-type cell.

As used herein the term “vector” is defined as a linear or circular DNA molecule comprising a polynucleotide encoding a polypeptide of the invention, and which is operably linked to additional nucleotides that ensure its expression.

“Introducing” in the context of a yeast cell means contacting a nucleic acid molecule with the cell in such a manner that the nucleic acid molecule gains access to the interior of the cell. Accordingly, polynucleotides and/or nucleic acid molecules can be introduced yeast cells in a single transformation event, in separate transformation events. Thus, the term “transformation” as used herein refers to the introduction of a heterologous nucleic acid into a cell. Transformation of a yeast cell can be stable or transient.

“Transient transformation” in the context of a polynucleotide means that a polynucleotide is introduced into the cell and does not integrate into the genome of the cell.

By “stably introducing” or “stably introduced” in the context of a polynucleotide introduced into a cell, it is intended that the introduced polynucleotide is stably incorporated into the genome of the cell, and thus the cell is stably transformed with the polynucleotide. “Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. Stable transformation as used herein can also refer to a nucleic acid molecule that is maintained extrachromosomally, for example, as a minichromosome.

Transient transformation may be detected by, for example, an enzyme-linked immunosorbent assay (ELISA) or Western blot, which can detect the presence of a peptide or polypeptide encoded by one or more nucleic acid molecules introduced into an organism. Stable transformation of a cell can be detected by, for example, a Southern blot hybridization assay of genomic DNA of the cell with nucleic acid sequences which specifically hybridize with a nucleotide sequence of a nucleic acid molecule introduced into an organism (e.g., a yeast). Stable transformation of a cell can be detected by, for example, a Northern blot hybridization assay of RNA of the cell with nucleic acid sequences which specifically hybridize with a nucleotide sequence of a nucleic acid molecule introduced into a yeast or other organism. Stable transformation of a cell can also be detected by, e.g., a polymerase chain reaction (PCR) or other amplification reaction as are well known in the art, employing specific primer sequences that hybridize with target sequence(s) of a nucleic acid molecule, resulting in amplification of the target sequence(s), which can be detected according to standard methods Transformation can also be detected by direct sequencing and/or hybridization protocols well known in the art.

Embodiments of the present invention also encompass variants of the polypeptides as defined herein. As used herein, a “variant” means a polypeptide in which the amino acid sequence differs from the base sequence from which it is derived in that one or more amino acids within the sequence are substituted for other amino acids. For example, a variant of SEQ ID NO: 1 may have an amino acid sequence at least about 50% identical to SEQ ID NO: 1, for example, at least about 80%, 85%, 90%, 91%, 92%, 93%, 94%, 95%, 96%, 97%, 98%, 99% or about 100% identical. The variants and/or fragments are functional variants/fragments in that the variant sequence has similar or identical functional enzyme activity characteristics to the enzyme having the non-variant amino acid sequence specified herein (and this is the meaning of the term “functional variant” as used throughout this specification).

A “functional variant” or “functional fragment” of any of the presented amino acid sequences, therefore, is any amino acid sequence which remains within the same enzyme category (i.e., has the same EC number) as the non-variant sequences. Methods of determining whether an enzyme falls within a particular category are well known to the skilled person, who can determine the enzyme category without use of inventive skill. Suitable methods may, for example, be obtained from the International Union of Biochemistry and Molecular Biology.

Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows: Class Amino Acid Examples
Nonpolar: A, V, L, I, P, M, F, W
Uncharged polar: G, S, T, C, Y, N, Q
Acidic: D, E
Basic: K, R, H.

As it is well known to those skilled in the art, altering the primary structure of a polypeptide by a conservative substitution may not significantly alter the activity of that polypeptide because the side-chain of the amino acid which is inserted into the sequence may be able to form similar bonds and contacts as the side chain of the amino acid which has been substituted out. This is so even when the substitution is in a region which is critical in determining the polypeptide's conformation.

In embodiments of the present invention, non-conservative substitutions are possible provided that these do not interrupt the enzyme activities of the polypeptides, as defined elsewhere herein. The substituted versions of the enzymes must retain characteristics such that they remain in the same enzyme class as the non-substituted enzyme, as determined using the NC-IUBMB nomenclature discussed above.

Broadly speaking, fewer non-conservative substitutions than conservative substitutions will be possible without altering the biological activity of the polypeptides. Determination of the effect of any substitution (and, indeed, of any amino acid deletion or insertion) is wholly within the routine capabilities of the skilled person, who can readily determine whether a variant polypeptide retains the enzyme activity according to aspects of the invention. For example, when determining whether a variant of the polypeptide falls within the scope of the invention (i.e., is a “functional variant or fragment” as defined above), the skilled person will determine whether the variant or fragment retains the substrate converting enzyme activity as defined with reference to the NC-IUBMB nomenclature mentioned elsewhere herein. All such variants are within the scope of the invention.

Using the standard genetic code, further nucleic acid sequences encoding the polypeptides may readily be conceived and manufactured by the skilled person, in addition to those disclosed herein. The nucleic acid sequence may be DNA or RNA, and where it is a DNA molecule, it may for example comprise a cDNA or genomic DNA. The nucleic acid may be contained within an expression vector, as described elsewhere herein.

Embodiments of the invention, therefore, encompass variant nucleic acid sequences encoding the polypeptides contemplated by embodiments of the invention. The term “variant” in relation to a nucleic acid sequence means any substitution of, variation of, modification of, replacement of, deletion of, or addition of one or more nucleotide(s) from or to a polynucleotide sequence, providing the resultant polypeptide sequence encoded by the polynucleotide exhibits at least the same or similar enzymatic properties as the polypeptide encoded by the basic sequence. The term includes allelic variants and also includes a polynucleotide (a “probe sequence”) which substantially hybridizes to the polynucleotide sequence of embodiments of the present invention. Such hybridization may occur at or between low and high stringency conditions. In general terms, low stringency conditions can be defined as hybridization in which the washing step takes place in a 0.330-0.825 M NaCl buffer solution at a temperature of about 40-48° C. below the calculated or actual melting temperature (Tm) of the probe sequence (for example, about ambient laboratory temperature to about 55° C.), while high stringency conditions involve a wash in a 0.0165-0.0330 M N buffer solution at a temperature of about 5-10° C. below the calculated or actual Tm of the probe sequence (for example, about 65° C.). The buffer solution may, for example, be a saline-sodium citrate (SSC) buffer (0.15M NaCl and 0.015M tri-sodium citrate), with the low stringency wash taking place in 3×SSC buffer and the high stringency wash taking place in 0.1×SSC buffer. Steps involved in hybridization of nucleic acid sequences have been described for example in Molecular Cloning, a laboratory manual [second edition] Sambrook et al. Cold Spring Harbor Laboratory, 1989, for example in Section 11 “Synthetic Oligonucleotide Probes” thereof.

Preferably, nucleic acid sequence variants have about 80% or more of the nucleotides in common with the nucleic acid sequence of embodiments of the present invention, more preferably at least 85%, or even 90%, 91%, 92%, 93%, 94%, 95%, 96%, 97%, 98% or 99% or greater sequence identity.

Variant nucleic acids of the invention may be codon-optimized for expression in a particular host cell.

As used herein, “sequence identity” refers to sequence similarity between two nucleotide sequences or two peptide or protein sequences. The similarity is determined by sequence alignment to determine the structural and/or functional relationships between the sequences.

Sequence identity between amino acid sequences can be determined by comparing an alignment of the sequences using the Needleman-Wunsch Global Sequence Alignment Tool available from the National Center for Biotechnology Information (NCBI), Bethesda, Md., USA, for example via http://blast.ncbi.nlm.nih.gov/Blast.cgi, using default parameter settings (for protein alignment, Gap costs Existence: 11 Extension: 1). Sequence comparisons and percentage identities mentioned in this specification have been determined using this software. When comparing the level of sequence identity to, for example, SEQ ID NO: 1, this, preferably should be done relative to the whole length of SEQ ID NO: 1 (i.e., a global alignment method is used), to avoid short regions of high identity overlap resulting in a high overall assessment of identity. For example, a short polypeptide fragment having, for example, five amino acids might have a 100% identical sequence to a five amino acid region within the whole of SEQ ID NO: 1, but this does not provide a 100% amino acid identity unless the fragment forms part of a longer sequence which also has identical amino acids at other positions equivalent to positions in SEQ ID NO: 1. When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences, to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties. As mentioned above, the percentage sequence identity may be determined using the Needleman-Wunsch Global Sequence Alignment tool, using default parameter settings. The Needleman-Wunsch algorithm was published in J. Mol. Biol. (1970) vol. 48: 443-453.

An aspect of the invention relates to a yeast cell capable of producing glutathione-polyamine conjugates. The yeast cell is capable of producing at least one polyamine and the yeast cell comprises a polyamine:glutathione ligase encoding gene and at least one polyamine synthase encoding gene but lacks a polyamine oxidase encoding gene or comprises a disrupted polyamine oxidase encoding gene.

In an embodiment, the yeast cell is engineered for overexpression of the polyamine: glutathione ligase.

The overexpression of the polyamine:glutathione ligase is, in an embodiment, achieved by putting the polyamine:glutathione ligase encoding gene under transcriptional control of a promoter that is highly active in the yeast cell. Suitable promoters for use in yeast cells include, but are not limited to, the promoters of PDC, GPD, GPD1, TEF1, PGK1, TDH and TDH3. Other suitable promoters include the promoters of GAL1, GAL2, GAL10, GAL7, CUP1, HIS3, CYC1, ADH1, PGL, GAPDH, ADC1, URA3, TRP1, LEU2, TPI, AOX1 and ENOI.

The yeast cell can comprise one or multiple, i.e., at least two, copies of the polyamine: glutathione ligase encoding gene to thereby increase the copy number of the mRNA for the polyamine: glutathione ligase and thereby the amount of polyamine:glutathione ligase produced by the yeast cell. In such a case, the multiple copies of the polyamine:glutathione ligase encoding gene could be under transcription control of one promoter, or each polyamine:glutathione ligase encoding gene could be under transcription control of a respective promoter. In the latter case, a same type of promoter could be used to control transcription of the respective polyamine:glutathione ligase encoding genes or different types of promoters could be used.

In an embodiment, the glutathione-polyamine conjugates are selected from the group consisting of trypanothione (N¹, N¹⁰-bis(glutathionyl) spermidine), N¹-glutathionyl spermidine, N¹⁰-glutathionyl spermidine, N¹,N¹⁰-bis(glutathionyl) spermine, N¹,N⁵, N¹⁰-tri(glutathionyl) spermine, N¹,N⁵,N¹⁰,N¹⁴-tetra(glutathionyl) spermine. Hence, the conjugate could be a conjugate between one polyamine, such as spermidine or spermine, and one, two or more, such as three or four, glutathione molecules.

In an embodiment, the polyamine:glutathione ligase encoding gene is selected from the group consisting of trypanothione synthase (EC 6.3.1.9), glutathionylspermidine synthase (EC 6.3.1.8) and a combination thereof.

In an embodiment, the trypanothione synthase (TryS) encoding gene is selected from the group consisting of Trypanosoma brucei brucei trypanothione synthase (TbbTryS) and a nucleotide sequence encoding a trypanothione synthase having at least 80% sequence identity with trypanothione synthase TbbTrS. In an embodiment, the nucleotide sequence encodes a trypanothione synthase having at least 85%, or even 90%, 91%, 92%, 93%, 94%, 95%, 96%, 97%, 98% or 99% sequence identity with Trypanosoma brucei brucei TrrS. In an embodiment, this trypanothione synthase having at least 80% sequence identity is capable of catalyzing conversion of glutathione and a polyamine into a glutathionylpolyamine and/or conversion of glutathione and a glutathionylpolyamine into a bis(glutathionyl) polyamine, preferably capable of catalyzing conversion of glutathione and spermidine into glutathionylspermidine and conversion of glutathione and glutathionylspermidine into N¹,N¹⁰-bis(glutathionyl) spermidine. The enzymatic efficacy of the trypanothione synthase having at least 80% sequence identity may be lower, substantially equal to or higher than the corresponding enzymatic efficacy of TbbTrrS, preferably at least substantially equal to or higher enzymatic efficacy.

The amino acid sequence for TbbTryS is shown in SEQ ID NO: 34 and the nucleotide sequence for Tbb TrrS is shown in SEQ ID NO: 35.

In an embodiment, the glutathionylspermidine synthase (GSS) encoding gene is selected from the group consisting of Escherichia coli glutathionylspermidine synthase (EcGSS) and a nucleotide sequence encoding a glutathionylspermidine synthase having at least 80% sequence identity with glutathionylspermidine synthase EcGSS. In an embodiment, the nucleotide sequence encodes a glutathionylspermidine synthase having at least 85%, or even 90%, 91%, 92%, 93%, 94%, 95%, 96%, 97%, 98% or 99% sequence identity with Escherichia coli EcGSS. In an embodiment, this glutathionylspermidine synthase having at least 80% sequence identity is capable of catalyzing conversion of glutathione and a polyamine into a glutathionylpolyamine, preferably capable of catalyzing conversion of glutathione and spermidine into glutathionylspermidine. The enzymatic efficacy of the glutathionylspermidine synthase synthase having at least 80% sequence identity may be lower, substantially equal to or higher than the corresponding enzymatic efficacy of EcGSS, preferably at least substantially equal to or higher enzymatic efficacy.

The amino acid sequence for EcGSS is shown in SEQ ID NO: 259 and the nucleotide sequence for EcGSS is shown in SEQ ID NO: 260.

In an embodiment, the at least one polyamine is selected from the group consisting of spermine, thermospermine, sym-homospermidine, 1,3-diaminopropane, putrescine, cadaverine, agmatine, spermidine, sym-norspermidine, norspermine and a combination thereof.

The yeast cell of the invention lacks a polyamine oxidase (EC 1.5.3.17) encoding gene or comprises a disrupted polyamine oxidase encoding gene. The yeast cell also comprises at least one polyamine synthase encoding gene.

The at least one polyamine synthase expressed by the yeast cell catalyzes the production of at least one polyamine in the yeast cell. Polyamine oxidase is an enzyme that catalyzes the conversion of spermine back to spermidine. Hence, the yeast cell lacks any polyamine oxidase encoding gene or comprises a disrupted polyamine oxidase encoding gene. This means that the yeast cell preferably lacks any polyamine oxidase or, if such a polyamine oxidase is expressed in the yeast cell, the polyamine oxidase is preferably enzymatically inactive or at least has significantly lower enzymatic efficiency as compared to the native polyamine oxidase.

In an embodiment, the yeast cell is engineered for overexpression of the at least one polyamine synthase.

The overexpression of the at least one polyamine synthase is, in an embodiment, achieved by putting the at least one at least one polyamine synthase encoding gene under transcriptional control of a promoter that is highly active in the yeast cell. Suitable promoters for use in yeast cells include, but are not limited to, the promoters of PDC, GPD, GPD1, TEF1, PGK1, TDH and TDH3. Other suitable promoters include the promoters of GAL1, GAL2, GAL10, GAL7, CUP1, HIS3, CYC1, ADH1, PGL, GAPDH, ADC1, URA3, TRP1, LEU2, TPI, AOX1 and ENOI.

The yeast cell can comprise one or multiple copies of the polyamine synthase encoding gene to thereby increase the copy number of the mRNA for the polyamine synthase and thereby the amount of polyamine synthase produced by the yeast cell. In such a case, the multiple copies of the polyamine synthase encoding gene could be under transcription control of one promoter, or each polyamine synthase encoding gene could be under transcription control of a respective promoter. In the latter case, a same type of promoter could be used to control transcription of the respective polyamine synthase encoding genes or different types of promoters could be used.

In an embodiment, the polyamine synthase encoding gene is selected from the group consisting of a spermine synthase (EC 2.5.1.22) encoding gene, a thermospermine synthase (EC 2.5.1.79) encoding gene and a homospermidine synthase (EC 2.5.1.44 or EC 2.5.1.45) encoding gene.

In an embodiment, the spermine synthase encoding gene is selected from the group consisting of Saccharomyces cerevisiae spermine synthase, preferably ScSPE4, Arabidopsis thaliana spermine synthase (AtSPMS) and a nucleotide sequence encoding a spermine synthase having at least 80% sequence identity with spermine synthase ScSPE4 or spermine synthase AtSPMS. In an embodiment, the nucleotide sequence encodes a spermine synthase having at least 85%, or even 90%, 91%, 92%, 93%, 94%, 95%, 96%, 97%, 98% or 99% sequence identity with ScSPE4 or AtSPMS. In an embodiment, this spermine synthase having at least 80% sequence identity is capable of catalyzing conversion of spermidine into spermine. The enzymatic efficacy of the spermine synthase having at least 80% sequence identity may be lower, substantially equal to or higher than the corresponding enzymatic efficacy of ScSPE4 or AtSPES, preferably at least substantially equal to or higher enzymatic efficacy.

The amino acid sequence for ScSPE4 is shown in SEQ ID NO: 1 and the nucleotide sequence for ScSPE4 is shown in SEQ ID NO: 2. The corresponding amino acid sequence for AtSPMS is shown in SEQ ID NO: 3 and the nucleotide sequence of AtSPMS is shown in SEQ ID NO: 4.

In an embodiment, the thermospermine synthase encoding gene is selected from the group consisting of Arabidopsis thaliana thermospermine synthase, preferably AtACL5, and a nucleotide sequence encoding a thermospermine synthase having at least 80% sequence identity with thermospermine synthase AtACL5. In an embodiment, the nucleotide sequence encodes a thermospermine synthase having at least 85%, or even 90%, 91%, 92%, 93%, 94%, 95%, 96%, 97%, 98% or 99% sequence identity with AtACL5. In an embodiment, this thermospermine synthase having at least 80% sequence identity is capable of catalyzing conversion of spermidine into thermospermine. The enzymatic efficacy of the thermospermine synthase having at least 80% sequence identity may be lower, substantially equal to or higher than the corresponding enzymatic efficacy of AtACL5, preferably at least substantially equal to or higher enzymatic efficacy.

The amino acid sequence for AtACL5 is shown in SEQ ID NO: 5 and the nucleotide sequence for AtACL5 is shown in SEQ ID NO: 6.

In an embodiment, the homospermidine synthase (HSS) encoding gene is selected from the group consisting of Senecio vernalis homospermidine synthase (SvHSS), Blastochloris viridis homospermidine synthase (BvHSS) and a nucleotide sequence encoding a homospermidine synthase having at least 80% sequence identity with homospermidine synthase SvHSS or homospermidine synthase BvHSS. In an embodiment, the nucleotide sequence encodes a homospermidine synthase having at least 85%, or even 90%, 91%, 92%, 93%, 94%, 95%, 96%, 97%, 98% or 99% sequence identity with SvHSS or BvHSS. In an embodiment, this homospermidine synthase having at least 80% sequence identity is capable of catalyzing conversion of putrescine into sym-homospermidine, or putrescine or spermidine into sym-homospermidine. The enzymatic efficacy of the homospermidine synthase having at least 80% sequence identity may be lower, substantially equal to or higher than the corresponding enzymatic efficacy of SvHSS or BvHSS, preferably at least substantially equal to or higher enzymatic efficacy.

The amino acid sequence for SvHSS is shown in SEQ ID NO: 7 and the nucleotide sequence for SvHSS is shown in SEQ ID NO: 8. The corresponding amino acid sequence for BvHSS is shown in SEQ ID NO: 9 and the nucleotide sequence for BvHSS is shown in SEQ ID NO: 10.

In an embodiment, the yeast cell selected from a group consisting of Saccharomyces, Kluyveromyces, Zygosaccharomyces, Candida, Hanseniaspora, Pichia, Hansenula, Schizosaccharomyces, Trigonopsis, Brettanomyces, Debaromyces, Nadsonia, Lipomyces, Cryptococcus, Aureobasidium, Trichosporon, Rhodotorula, Yarrowia, Rhodosporidium, Phaffia, Schwanniomyces, Aspergillus and Ashbya. In a particular embodiment, the yeast cell is selected from the group consisting of Saccharomyces cerevisiae, Saccharomyces boulardii, Zygosaccharomyces bailii, Kluyveromyces lactis, Rhodosporidium toruloides, Yarrowia lipolytica, Schizosaccharomyces pombe, Pichia pastoris, Hansenula anomala, Candida sphaerica, or Schizosaccharomyces malidevorans. Saccharomyces cerevisiae is a preferred yeast species.

In an embodiment, the yeast cell is a Saccharomyces cerevisiae cell and the polyamine oxidase is FMS1. Hence, in an embodiment, the S. cerevisiae cell lacks FMS1 or comprises a disrupted FMS1.

Another aspect of the invention relates to a yeast cell capable of producing glutathione-polyamine conjugates. The yeast cell is capable of producing at least one polyamine and the yeast cell comprises a polyamine:glutathione ligase encoding gene.

Various embodiments of the yeast cell as described in the foregoings can also be applied to this aspect of the invention.

A further aspect of the invention relates to a method of producing glutathione-polyamine conjugates. The method comprising culturing a yeast cell according to the present invention in a culture medium and in culture conditions suitable for production of the glutathione-polyamine conjugates by the yeast cell. The method also comprises collecting the glutathione-polyamine conjugates from the culture medium and/or from the yeast cell.

The culture medium in this aspect of the invention can be any culture medium, in which the yeast cell can be cultured to produce glutathione-polyamine conjugates. The culturing can be in the form of, for instance, batch, fed-batch or perfusion culturing or fermentation, bioreactor fermentation, etc.

### EXAMPLES

### Example 1: Improvement of Spermidine Production by Systematic Rewiring of Native Metabolism in Yeast

In this Example 1, we systematically refactored metabolism in a yeast strain, including central carbon and nitrogen metabolism, methionine salvage pathway, salvage pathways of adenine, polyamine transport machinery, and polyamine consumption/degradation pathway. In addition, we also introduced extra potential positive genetic targets. This yeast strain built with a new modular genetic design. Specially, the de novo Spd biosynthetic pathway split into multiple genetic modules that contain the coding sequences for numerous biosynthetic enzymes to divert greater carbon flux from sugar carbon source to Spd.

A precursor overproduction module (I) designed to increase accumulation of L-ornithine (Orn) including the overexpression of eight proteins: NADP⁽⁺⁾-dependent glutamate dehydrogenase from S. cerevisiae (GDH1) [SEQ ID NO: 11], mitochondrial aspartate and glutamate carrier protein from S. cerevisiae (AGC1) [SEQ ID NO: 12], mitochondrial L-ornithine carrier protein from S. cerevisiae (ORT1) [SEQ ID NO: 13], glutamate N-acetyltransferase from E. coli (EcargA) [SEQ ID NO: 14], acetylglutamate kinase from E. coli (EcargB) [SEQ ID NO: 15], N-acetyl-gamma-glutamyl-phosphate reductase from Corynebacterium glutamicum (CgargC) [SEQ ID NO: 16], acetylornithine aminotransferase from C. glutamicum (CgargD) [SEQ ID NO: 17], and ornithine acetyltransferase from C. glutamicum (CgargJ) [SEQ ID NO: 18]. Moreover, attenuation or removal of two proteins was also included in this module (I): attenuation of yeast native ornithine carbamoyltransferase (ARG3) [SEQ ID NO: 19] by swapping its native promoter P_{ARG3} with weaker promoter P_{KEX2}, and removing the activity of L-ornithine transaminase (CAR2) [SEQ ID NO: 20] by knockout of CAR2.

A putrescine (Put) module (II) designed to overproduce Put from L-ornithine included two genetic modifications; the overexpression of ornithine decarboxylase from S. cerevisiae (SPE1) [SEQ ID NO: 21] and the deletion of native ornithine decarboxylase antizyme (OAZ1) [SEQ ID NO: 22].

A spermidine biosynthesis module (III) was designed for overproduction of spermidine (Spd) from putrescine and was characterized by overexpression of two proteins from S. cerevisiae: adenosylmethionine decarboxylase (AdoMetDC; SPE2) [SEQ ID NO: 23] and spermidine synthase (SpdSyn; SPE3) [SEQ ID NO: 24]. This module also included the deletion of two native proteins to avoid spermidine consumption or degradation: deletion of SPE4 [SEQ ID NO: 2] encoding spermine synthase and FMS1 [SEQ ID NO: 25] encoding a non-specific polyamine oxidase.

An S-adenosyl-L-methionine (AdoMet) module (IV) was designed to increase the accessibility of co-factor AdoMet. These modifications included overexpression of numerus proteins: 5′-methylthioadenosine phosphorylase (MEU1) [SEQ ID NO: 26] from S. cerevisiae, branched-chain amino acid aminotransferase (BAT2) [SEQ ID NO: 27] from S. cerevisiae, adenine phosphoribosyltransferase (APT1) [SEQ ID NO: 28] from S. cerevisiae, ribose-phosphate pyrophosphokinases (PRS5) [SEQ ID NO: 29] from S. cerevisiae, and S-adenosylmethionine synthase from Leishmania infantum (LiMAT) [SEQ ID NO: 30]. This module also included deletion of adenine deaminase activity (AAH1) [SEQ ID NO: 31].

A polyamine efflux module (V) was designed to relieve the cytotoxicity to the cell or the inhibition to the polyamine biosynthesis. This module included the overexpression of yeast native polyamine transporter encoded by TPO5 [SEQ ID NO: 32].

Finally yet importantly, an extra spermidine biosynthesis module (VI) was designed for overproduction of spermidine from putrescine and AdoMet. This module included overexpression of an AdoMetDC-SpdSyn fusion protein encoded by SPE2-SPE3 [SEQ ID NO: 33].

The overexpression of genes in this Example 1 was obtained by chromosomal integrations to the regions from which we expected no growth defect and active expression as integration loci via the CRISPR/cas9 system or the traditional genetic makers based methods. The implementation of CRISPR/cas9 based genome editing followed the protocol developed by Mans et al. 2015. In particular, S. cerevisiae strain CEN.PK113-11C harboring plasmid pL-CAS9-HIS with HIS3 marker, enabling the constitutive expression of Cas9, was the start strain for all genetic manipulations. To enable the efficient genome editing in the selected loci, multiple guide RNA (gRNA) plasmids were constructed. Genetic modules, including various combinations of genetic parts, i.e., promoters, terminators, ORFs and homologous arms constructed as integration cassettes according to overlapping extension PCR (OE-PCR) procedure. The following gene and promoter combinations where used in this Example 1: TPI1p-ORT1-pYX212t; tHXT7p-AGC1-CYC1t; TEF1p-GDH1-DIT1t; PGK1p-SPE3-pYX212t; TEF1p-SPE1-PRM9t; TDH3p-SPE2-DIT1t; TDH3p-CgargJ-TDH2t; PGK1p-EcargB-ADH1t; TEF1p-CgargC-FBA1t; tHXT7p-CgargD-TPI1t; TPI1p-EcargA-CYC1t; TPI1p-MEU1p-FBA1t; PGK1p-BAT2-CYC1t; TDH3p-APT1-DIT1t; TEF1p-PRS5-PRM9t; TEF1p-LiMAT-PRM9; TDH3p-TPO5-CYC1t; TEF1p-SPE2-SPE3-PRM9t.

All the native genetic parts, i.e., native promoters, terminators, ORFs and homologous arms, were PCR amplified using CEN.PK113-11C genomic DNA as a template. For optimized heterologous genes, synthetic fragments or plasmids (obtained from GenScript) used for PCR amplification. High-fidelity Phusion DNA polymerase utilized throughout the entire molecular cloning procedure. Cassettes or plasmids introduced into the yeast by the standard LiAc/SS DNA/PEG transformation method. Strains containing URA3-based plasmids or cassettes selected on synthetic complete media without uracil medium (SC-URA), which consisted of 6.7 g/l yeast nitrogen base (YNB) without amino acids, 0.77 g/l complete supplement mixture without uracil (CSM-URA), 20 g/l glucose and 20 g/l agar. The URA3 maker was removed and selected against on 5-fluoroorotic acid (5′-FOA) plates. Moreover, CRISPR/cas9 based system was also used to practice the deletions of AAH1, SPE4 and FMS1. The other gene knockout experiments were conducted by the traditional methods. All the primers used herein are listed in Table 1, all plasmids are listed in Table 2, and all strains are listed in Table 3.

TABLE 1
primers

| | | SEQ |
|---|---|---|
| | | ID |
| Oligo Name | Sequence 5′ to 3′ | NO: |
| 001_OAZ1- | GTATGAAGTAATACAGAAAAGGAAAAC | 36 |
| L-F1 | | |
| OAZ1-L-R1 | GACCTGCAGCGTACGAAGCTTCAGCAATCTCT | 37 |
| (Kan) | GGTGGAACGTCTAG | |
| KanMX-F1 | CTGAAGCTTCGTACGCTGCAGGTC | 38 |
| KanMX-R1 | GGCCACTAGTGGATCTGATATCAC | 39 |
| OAZ1-R- | GTGATATCAGATCCACTAGTGGCCCCTGAACC | 40 |
| F1(kan) | AAAACTTCAAATTCGAATAC | |
| OAZ1-R-R1 | GCATTCAAACTCTAAAATAACAAAG | 41 |
| TEF1P- | GTGATATCAGATCCACTAGTGGCCATAGCTTC | 42 |
| F1(kan) | AAAATGTTTCTACTCC | |
| 008_TEF1p- | TTTGTAATTAAAACTTAGATTAGATTGC | 43 |
| R1 | | |
| PR9_SPE1- | GCAATCTAATCTAAGTTTTAATTACAAAATGT | 44 |
| F1(TEF) | CTAGTACTCAAGTAGGAAATGC | |
| PR10_SPE1- | GTGCTAGTGTCTCCCGTCTTCTGTTCAATCGA | 45 |
| R1(PRM9) | GTTCAGAGTCTATGTATAC | |
| PR_11_PR | ACAGAAGACGGGAGACACTAGCAC | 46 |
| M9t-F1 | | |
| 12_PRM9t- | ATTTTCAACATCGTATTTTCCGAAGC | 47 |
| R1 | | |
| OAZ1-R- | GCTTCGGAAAATACGATGTTGAAAATCCTGAA | 48 |
| F2(PRM9) | CCAAAACTTCAAATTCGAATAC | |
| pyx212t- | GTGATATCAGATCCACTAGTGGCCTGCCGTAA | 49 |
| R1(kan) | ACCACTAAATCGGAACCC | |
| 15_pyx212t- | TAGGGCCCACAAGCTTACGCGTCGAC | 50 |
| F1 | | |
| SPE3- | GTCGACGCGTAAGCTTGTGGGCCCTACTAATT | 51 |
| R1(pyx212t) | TAATTCCTTGGCTGCCCAG | |
| SPE3- | CTACTTTTTACAACAAATATAACAAAATGGCA | 52 |
| F1(PGK) | CAAGAAATCACTCACCCAAC | |
| 18_PGK1p- | TTTGTTATATTTGTTGTAAAAAGTAG | 53 |
| R1 | | |
| PGK1p-F1 | ACGCACAGATATTATAACATCTGCAC | 54 |
| TEF1p- | GTGCAGATGTTATAATATCTGTGCGTATAGCT | 55 |
| F2(PGK1p) | TCAAAATGTTTCTACTCC | |
| DIT1t- | CTTCGGAAAATACGATGTTGAAAATGTTACTC | 56 |
| R1(PRM9t) | CGCAACGCTTTTCTGAACG | |
| PR22_DIT1t- | TAAAGTAAGAGCGCTACATTGGTCTACC | 57 |
| F1 | | |
| SPE2- | GGTAGACCAATGTAGCGCTCTTACTTTATCAT | 58 |
| R1(DIT1t) | ATTTTCTTCTGCAATTTCATATAG | |
| SPE2- | GTTTCGAATAAACACACATAAACAAACAAAAT | 59 |
| F1(TDH3p) | GACTGTCACCATAAAAGAATTGAC | |
| PR25_TDH3 | TTTGTTTGTTTATGTGTGTTTATTCGAAAC | 60 |
| p-R1 | | |
| 26_TDH3p- | TCGAGTTTATCATTATCAATACTGCC | 61 |
| F1 | | |
| OAZ1-R- | GGCAGTATTGATAATGATAAACTCGACCTGAA | 62 |
| F3(TDH3p) | CCAAAACTTCAAATTCGAATAC | |
| TPlp- | GGCAGTATTGATAATGATAAACTCGAGTTTAA | 63 |
| F1(TDH3p) | AGATTACGGATATTTAACTTAC | |
| PR29_TPIp- | TTTTAGTTTATGTATGTGTTTTTTGTAGTTAT | 64 |
| R1 | AG | |
| SPE4- | CTATAACTACAAAAAACACATACATAAACTAA | 65 |
| F1(TPlp) | AAATGGTTAATAATTCACAGCATCCTTAC | |
| SPE4- | GACTAATAATTCTTAGTTAAAAGCACTTCATT | 66 |
| R1(ENO2t) | CATTAATGACCTTGTCTGCCC | |
| ENO2t-F1 | AGTGCTTTTAACTAAGAATTATTAGTC | 67 |
| ENO2t-R1 | AGGTATCATCTCCATCTCCCATATGC | 68 |
| OAZ1-R- | GCATATGGGAGATGGAGATGATACCTCCTGAA | 69 |
| F4(ENO2t) | CCAAAACTTCAAATTCGAATAC | |
| (ZWM)TPlp- | CGTCCTCTCGAAAGGTGGTTTAAAGATTACGG | 70 |
| F-156 | ATATTTAACTTAC | |
| (TPlp)MEU1- | CTACAAAAAACACATACATAAACTAAAAATGA | 71 |
| F-157 | ACAGGATTAAGAATACATTTTCTG | |
| MEU1- | GGTCGACGCGTAAGCTTGTGGGCCCTATTACC | 72 |
| R(Pyx212t)- | AATAGTTTGGAAATAAGTATC | |
| 158 | | |
| Pyx212t- | CAGGTGGTCATGGCCCTTTGCCGTAAACCACT | 73 |
| R(ZWM)- | AAATCG | |
| 159 | | |
| (TPlp)BAT2- | CTACAAAAAACACATACATAAACTAAAAATGA | 74 |
| F-160 | CCTTGGCACCCCTAGAC | |
| BAT2- | GGTCGACGCGTAAGCTTGTGGGCCCTATCAGT | 75 |
| R(Pyx212t)- | TCAAATCAGTAACAACC | |
| 161 | | |
| MEU1- | CATTAAAAAACTATATCAATTAATTTGAATTA | 76 |
| R(FBA1t)- | ACTTACCAATAGTTTGGAAATAAGTATC | |
| 162 | | |
| BAT2- | CATGACTCGAGGTCGACGGTATCTCAGTTCAA | 77 |
| R(CYC1t)- | ATCAGTAACAACCCTTG | |
| 163 | | |
| BAT2- | GTAATTATCTACTTTTTACAACAAATATAACA | 78 |
| F(PGK1p)- | AAATGACCTTGGCACCCCTAGAC | |
| 164 | | |
| TEF1p- | CAGGTGGTCATGGCCCTTTTTGTAATTAAAAC | 79 |
| R(ZWM)- | TTAGATTAGATTGC | |
| 165 | | |
| (TEF1p)SA | GCAATCTAATCTAAGTTTTAATTACAAAATGT | 80 |
| M2-F-166 | CCAAGAGCAAAACTTTCTTATTTACC | |
| SAM2- | CTAAATCATTAAAGTAACTTAAGGAGTTAAAT | 81 |
| R(TDH2t)- | TTAAAATTCCAATTTCTTTGG | |
| 167 | | |
| TDH2t-F- | ATTTAACTCCTTAAGTTACTTTAATGATTTAG | 82 |
| 168 | | |
| ADH1t- | CAGGTGGTCATGGCCCTTGCGAATTTCTTATG | 83 |
| F(ZWM)-169 | ATTTATG | |
| ADH1t-F- | GCGAATTTCTTATGATTTATG | 84 |
| 170 | | |
| APT1- | CATAAATCATAAGAAATTCGCTCATTTTTTCA | 85 |
| R(ADH1t)- | ACGCTTCCTTTTGAGC | |
| 171 | | |
| APT1- | CGAATAAACACACATAAACAAACAAAATGTCT | 86 |
| F(TDH3p)- | ATAGCAAGTTATGCCCAAG | |
| 172 | | |
| tHXT7p- | CAGGTGGTCATGGCCCTTTTTTTGATTAAAAT | 87 |
| R(ZWM)- | TAAAAAAACTTTTTG | |
| 173 | | |
| tHXT7p-R- | TTTTTGATTAAAATTAAAAAAACTTTTTG | 88 |
| 174 | | |
| PRS5- | CAAAAAGTTTTTTTAATTTTAATCAAAAAATG | 89 |
| F(tHXT7p)- | TCAATGAGTAATATTGTTGTTTTTGG | |
| 175 | | |
| (ZWM)FBA1 | CGTCCTCTCGAAAGGTGGTTAATTCAAATTAA | 90 |
| t(F)-176 | TTGATATAGTTTTTTAATG | |
| p423GPD1- | AAGGGCCATGACCACCTGATGCACCAATTAGG | 91 |
| F1-177 | TAGGTCTGGCTATGTCTATACCTCTGGCAATT | |
| | CGCCCTATAGTGAGTCG | |
| p423GPD1- | CACCTTTCGAGAGGACGATGCCCGTGTCTAAA | 92 |
| R1-178 | TGATTCGACCAGCCTAAGAATGTTCAACGAGC | |
| | TCCAGCTTTTGTTCCCTTTAGTG | |
| (ZWM)TDH3 | GCATCGTCCTCTCGAAAGGTGTCGAGTTTATC | 93 |
| p-179 | ATTATCAATACTGCC | |
| TDH2t-F1- | ATTTAACTCCTTAAGTTACTTTAATGATTTAG | 94 |
| 182 | | |
| ADH1t-F1- | GCGAATTTCTTATGATTTATG | 95 |
| 183 | | |
| TPI1t-F1- | GATTAATATAATTATATAAAAATATTATCTTC | 96 |
| 188 | TTTTC | |
| PAA1-L-F- | CTGGTTTGTTTTACAACCAAAAG | 97 |
| 211 | | |
| PAA1-L- | CAACTCTTTCATAAAATGGTATCTTTAACTTT | 98 |
| R(LINKER)- | TTATTTAATCGTAATG | |
| 212 | | |
| Verify- | CAATGACAAGTTTCATCATC | 99 |
| PAA1-F-216 | | |
| 277- | GCAATCTAATCTAAGTTTTAATTACAAAGTTA | 100 |
| (TEF1p)DIT | CTCCGCAACGCTTTTCTGAACG | |
| 1t-R | | |
| 278- | GCAATCTAATCTAAGTTTTAATTACAAAATGA | 101 |
| (TEF1p)- | ACAGGATTAAGAATACATTTTCTG | |
| MEU1-F | | |
| 279-MEU1- | CTAGTGTCTCCCGTCTTCTGTTTACCAATAGT | 102 |
| R(PRM9t) | TTGGAAATAAGTATC | |
| 280-(TPlp)- | CTATAACTACAAAAAACACATACATAAACTAA | 103 |
| BAT2-F | AAATGACCTTGGCACCCCTAGAC | |
| 281-BAT2- | GACTAATAATTCTTAGTTAAAAGCACTTCAGT | 104 |
| R(ENO2t) | TCAAATCAGTAACAACC | |
| 282TaSPDS- | GTCGACGCGTAAGCTTGTGGGCCCTATCATGA | 105 |
| R1(pyx212t) | TGCTGTAATAGCAGAATC | |
| 283(PGK)Ta | CTACTTTTTACAACAAATATAACAAAATGGAA | 106 |
| SPDS-F1 | GGTGGTGGTGCTAGAAATG | |
| 290TPO5- | CTATAACTACAAAAAACACATACATAAACTAA | 107 |
| F1(TPlp) | AAATGCCAGAGTATACGCTACTG | |
| 291TPO5- | GACTAATAATTCTTAGTTAAAAGCACTTCATA | 108 |
| R1(ENO2t) | TATCATATCTACGATCATCG | |
| 2920AZ1- | GTGAAGGAACAACTCGTGTCTC | 109 |
| F(Veri) | | |
| 2930AZ1- | GAGGATACGTACATATGCAAGC | 110 |
| R(Veri) | | |
| 294_ | TGCCGTAAACCACTAAATCGGAACC | 111 |
| pYX212t-R | | |
| 295-CAN1- | TCAGACTTCTTAACTCCTGTAAAAACAAAAAA | 112 |
| FW | AAAAAAAGGCATAGCAATAAGCTGGAGCTCAT | |
| | AGCTTC | |
| 296-CYC1t- | GTGCCTATTGATGATCTGGCGGAATGTCTGCC | 113 |
| tagA-RV | GTGCCATAGCCATGCCTTCACATATAGTCCGC | |
| | AAATTAAAGCCTTCGAG | |
| 297-tagA- | ACTATATGTGAAGGCATGGCTATGGCACGGCA | 114 |
| PUG | GACATTCCGCCAGATCATCAATAGGCACCTTC | |
| | GTACGCTGCAGGTCGAC | |
| 315_SPE4_ | TGCGCATGTTTCGGCGTTCGAAACTTCTCCGC | 115 |
| targetRNA | AGTGAAAGATAAATGATCACTCATCAAATTCC | |
| FW | GTACATGTTTTAGAGCTAGAAATAGCAAGTTA | |
| | AAATAAGGCTAGTCCGTTATCAAC | |
| 316_SPE4_ | GTTGATAACGGACTAGCCTTATTTTAACTTGC | 116 |
| targetRNA | TATTTCTAGCTCTAAAACATGTACGGAATTTG | |
| RV | ATGAGTGATCATTTATCTTTCACTGCGGAGAA | |
| | GTTTCGAACGCCGAAACATGCGCA | |
| 317_SPE4_ | ATAAAATATCAAAACGCCGATGAGACAGGCAG | 117 |
| repair | GATAAAGTGACAGATTCAGTTATACATTTTTA | |
| oligo fw | TTAGCATTGATATTATTATTTTAAAAAGTCTA | |
| | TTTACTTGTATATTTATCCGAATA | |
| 318_SPE4_ | TATTCGGATAAATATACAAGTAAATAGACTTT | 118 |
| repair | TTAAAATAATAATATCAATGCTAATAAAAATG | |
| oligo rv | TATAACTGAATCTGTCACTTTATCCTGCCTGT | |
| | CTCATCGGCGTTTTGATATTTTAT | |
| 319_SPE4_ | TTTTGCAACATCCGGGCATG | 119 |
| dg fw | | |
| 320_SPE4_ | CGGCTAGCTGGTATGGATCG | 120 |
| dg rv | | |
| 321_TEF1p_ | ATAGCTTCAAAATGTTTCTACTCC | 121 |
| FW | | |
| 322_X-2- | TGCGACAGAAGAAAGGGAAG | 122 |
| Diag_FW | | |
| 323_X_2_ | CGTCTATGAGGAGACTGTTAG | 123 |
| UP_FW | | |
| 324_X_2_ | GGAGTAGAAACATTTTGAAGCTATACGTGACC | 124 |
| UP_RV | ACTTCGAGAGC | |
| (TEF1p) | | |
| 325_SPE2_ | GCAATCTAATCTAAGTTTTAATTACAAAATGA | 125 |
| FW(TEF1p) | CTGTCACCATAAAAGAATTG | |
| 326_SPE2_ | GTGCTAGTGTCTCCCGTCTTCTGTTCATATTT | 126 |
| RV(PRM9t) | TCTTCTGCAATTTCATATAG | |
| 327_X_2_ | GCTTCGGAAAATACGATGTTGAAAATCCTGCA | 127 |
| DW_FW | TAATCGGCCTCAC | |
| (PRM9t) | | |
| 328_X_2_ | CTCGCCAAGGCATTACCATC | 128 |
| DW_RV | | |
| 329_X-2- | GAGAACGAGAGGACCCAAC | 129 |
| Diag_RV | | |
| 330_ | GGTTCCGATTTAGTGGTTTACGGCAACGTGAC | 130 |
| X2_UP_RV | CACTTCGAGAGC | |
| (pYX212t) | | |
| 331_SPE2_ | GTCGACGCGTAAGCTTGTGGGCCCTATCATAT | 131 |
| RV(PYX212) | TTTCTTCTGCAATTTCATATAG | |
| 332_SPE2_ | CTACTTTTTACAACAAATATAACAAAATGACT | 132 |
| FW(PGK1p) | GTCACCATAAAAGAATTG | |
| 333_APT1_ | GGTAGACCAATGTAGCGCTCTTACTTTATCAT | 133 |
| RV(DIT1t) | TTTTTCAACGCTTCCTTTTG | |
| 334_APT1_ | GTTTCGAATAAACACACATAAACAAACAAAAT | 134 |
| FW(TDH3p) | GTCTATAGCAAGTTATGCCCAAG | |
| 335_X_2_ | GGCAGTATTGATAATGATAAACTCGACCTGCA | 135 |
| DW_FW | TAATCGGCCTCAC | |
| (TDH3p) | | |
| 336_PRS5_ | CTATAACTACAAAAAACACATACATAAACTAA | 136 |
| FW(TP11p) | AAATGTCAATGAGTAATATTGTTG | |
| 337_PRS5_ | GACTAATAATTCTTAGTTAAAAGCACTCTACC | 137 |
| RV(ENO2t) | ATCCATGCTCGAACAAC | |
| 338_X_2_ | GCATATGGGAGATGGAGATGATACCTCCTGCA | 138 |
| DW_FW | TAATCGGCCTCAC | |
| (ENO2t) | | |
| 359-CYC1t- | GGGTACCGGCCGCAAATTAAAGCCTTCGAGCG | 139 |
| RV | TCCC | |
| 392-X-4- | GTGTTCATTGTACGTCCTAGAC | 140 |
| up-dg-fw | | |
| 393-X-4- | GTGCCCAAAGCTAAGAGTC | 141 |
| up-fw | | |
| 394-X-4- | CTGCTCTTGAATGGCGAC | 142 |
| up-rv | | |
| 395-(X-4- | GTCGCCATTCAAGAGCAGCATCGTCCTCTCGA | 143 |
| up)YP8-fw | AAGGTG | |
| 396-(X-4- | CGAATCTTCCCATGCCTGCAGGTGGTCATGGC | 144 |
| down)YP8- | CCTT | |
| rv | | |
| 397-X-4- | CAGGCATGGGAAGATTCG | 145 |
| down-fw | | |
| 398-X-4- | CTGGTGAGGATTTACGGTATG | 146 |
| down-rv | | |
| 399-X-4- | GTGCGTTATCGGGTTCTTAC | 147 |
| down-dg-rv | | |
| 400-XI-3- | CAGGTTAGTTACTTGCTCTATG | 148 |
| up-fw | | |
| 401-XI-3- | CAGTATTGATAATGATAAACTCGAAATCAGAC | 149 |
| up-rv | GCACGCTTG | |
| (TDH3p) | | |
| 402- | CTTTAATTTGCGGCCGGTACCCTTACGTGGAT | 150 |
| (CYC1t)XI- | TGAGCCAG | |
| 3-dow-fw | | |
| 403-XI-3- | GATTGTCATAATAGGAGCTATTTG | 151 |
| dow-rv | | |
| 404-XI-3- | CCATAGTATTACTATTGGTGTTCAT | 152 |
| up-fw(dg) | | |
| 405-XI-3- | GTTATCGGTTGTGATATTGTTC | 153 |
| down-rv(dg) | | |
| 406- | TTAAGCTATTGTTTCGGCAATT | 154 |
| CgargD-rv | | |
| 425-CAR2p- | CGTGCGAUCTCTATAAAAAATGTGCGAAC | 155 |
| fw | | |
| 426-CAR2p- | ATGACAGAUTGGTGTTGTGGTTCTGTG | 156 |
| rv | | |
| 427-X-3-up- | GAGATCTTTGTGTTCGGTTAC | 157 |
| fw | | |
| 428-X-3-up- | AGTCTCGTATGTCGGCTC | 158 |
| rv | | |
| 431-X-3- | TGTGTCCGCGTTTCTAAG | 159 |
| down-fw | | |
| 432-X-3- | GAGGTGGTTATTGATCACCAG | 160 |
| down-rv | | |
| 433-(dg)X- | ACGAATCGTTAGGCACAG | 161 |
| 3-up-fw | | |
| 434-(dg)X- | GTGCAATACCAAAATCG | 162 |
| 3-down-rv | | |
| 435-GO4- | GCAGTTGTTTGGATTAAAAAGCTGTACG | 163 |
| dg1-fw | | |
| 436-GO4- | CCTTGTGTCATCATTTACTCCAGGC | 164 |
| dg1-rv | | |
| 437-GO4- | GTAGAGTCTTAGCTGCAGTTGGTATG | 165 |
| dg2-fw | | |
| 438-GO4- | CAGGGCATTATTACTGACGGCATG | 166 |
| dg2-rv | | |
| 439-GO4- | CTACAGCACCTTTGAAAGAAGGTGTC | 167 |
| dg3-fw | | |
| 440-GO4- | GTTGATGGTGTCGTAGACGTCAG | 168 |
| dg3-rv | | |
| 441-GO4- | GGAACGTGGATTTACCCCAG | 169 |
| dg4-fw | | |
| 442-GO4- | GTTATCGGTTGTGATATTGTTCCTGC | 170 |
| dg4-rv | | |
| 443-MEU1- | CAAAGCGATGGGCTCCAGAC | 171 |
| dg-rv | | |
| 444-BAT2- | CATTCCGCAGTTAACATGTGGTC | 172 |
| dg-fw | | |
| 445-X-4-up- | GTGTTCATTGTACGTCCTAGACTCAAAC | 173 |
| dg-fw2 | | |
| 446-X-4- | CGTGCGTTATCGGGTTCTTAC | 174 |
| down-dg-rv2 | | |
| 447-(X-4- | GGAACACTGGGGCAATAGGCTGTCGCCATTC | 175 |
| up)YP8-fw2 | AAGAGCAGCATCGTCCTCTCGAAAGGTG | |
| 448-(X-4- | CTATTGTAATTCAAAAAAAAAAAGCGAATCT | 176 |
| down)YP8- | TCCCATGCCTGCAGGTGGTCATGGCCCTT | |
| rv2 | | |
| 454_PGK1p- | CTTGCATAAATTGGTCAATGCAAG | 177 |
| Fw1(dg) | | |
| 455_TEF1p- | CGATGACCTCCCATTGATATTTAAG | 178 |
| Fw1(dg) | | |
| 456_SPE2 | CATCGTCAATTTGTGATCGAAGAC | 179 |
| Rv1(dg) | | |
| 457_APT1_ | CATTCGCCAGGTAGCTTAC | 180 |
| Rv1(dg) | | |
| 458_PRS5_ | TGCATTTTGAGCGTTGAACAA | 181 |
| Rv1(dg) | | |
| 459_TDH3p_ | GTGCCCTGTTCTCTGTAGTT | 182 |
| Rv1(dg) | | |
| 460_ | ATCGGGCCCTCCTTACTGCTCTCCTTCCGTGT | 183 |
| pYX212t_rv | AACGCGTTTGCCGTAAACCACTAAATCG | |
| (XII-2_up) | | |
| 465_PRM9t_ | CGTTAAGAAAAATTTCGAGAGAGTCGCCGATA | 184 |
| rv(XII- | GTAGATTTTCAACATCGTATTTTCC | |
| 2_down) | | |
| 466_TEF1p_ | CCTCCTTACTGCTCTCCTTCCGTGTAACGCGT | 185 |
| fw(XII- | TATAGCTTCAAAATGTTTCTACTCC | |
| 2_up) | | |
| 467_ XII- | TTATCGAGCTAACTATTTTCGACACACATG | 186 |
| 2_up_fw | | |
| 468_ XII- | CGTCGCCCAGTAAGTGAGACTA | 187 |
| 2_down_rv | | |
| 471_PRS5_ | GAAAGCATAGCAATCTAATCTAAGTTTTAATT | 188 |
| fw(TEF1p) | ACAAAATGTCAATGAGTAATATTGTTG | |
| 472_PRS5_ | AAGTTGTGTGCTAGTGTCTCCCGTCTTCTGTC | 189 |
| rv(PRM9t) | TACCATCCATGCTCGAACA | |
| 488_KEX2p_ | CTCGCCTAGTAAATAAACGATAAACAAATTTG | 190 |
| (PFK1p_ | AAGTAGTAGATACACGTATCTCGACATG | |
| up)_fw | | |
| 489_KEX2p_ | GAATGCAACACCGTAGCATGAATCTTGAGATT | 191 |
| (PFK1p)_rv | GCATCTGATAATGGGTTAGTAGTTTAT | |
| 496_FMS1_ | TCTCCGCAGTGAAAGATAAATGATCAATTTAC | 192 |
| targetRNA | GAAAAATAAAGGCGTTTTAGAGCTAGAAATAG | |
| FW | CAAGTT | |
| 497_FMS1_ | AACTTGCTATTTCTAGCTCTAAAACGCCTTTA | 193 |
| targetRNA | TTTTTCGTAAATTGATCATTTATCTTTCACTG | |
| RV | CGGAGA | |
| 498_FMS1_ | AATAAAGGCAAAAACAGTGGTCGTGTGAGAAA | 194 |
| repair | TCTATTTTTTCGAAATTACTTACACTTT | |
| oligo fw | | |
| 499_FMS1_ | AAAGTGTAAGTAATTTCGAAAAAATAGATTTC | 195 |
| repair | TCACACGACCACTGTTTTTGCCTTTATT | |
| oligo rv | | |
| 500_FMS1_ | GGTCACCCACCCATATACGG | 196 |
| dg fw | | |
| 501_FMS1_ | TGTCCTCCGGATAACTGCAC | 197 |
| dg rv | | |
| FMS1_ | TGCGCATGTTTCGGCGTTCGAAACTTCTCCGC | 198 |
| targetRNA | AGTGAAAGATAAATGATCAATTTACGAAAAAT | |
| FW | AAAGGCGTTTTAGAGCTAGAAATAGCAAGTTA | |
| | AAATAAGGCTAGTCCGTTATCAAC | |
| FMS1_ | GTTGATAACGGACTAGCCTTATTTTAACTTGC | 199 |
| targetRNA | TATTTCTAGCTCTAAAACGCCTTTATTTTTCG | |
| RV | TAAATTGATCATTTATCTTTCACTGCGGAGAA | |
| | GTTTCGAACGCCGAAACATGCGCA | |
| FMS1_repair | TCTTTTTTTGTTCCCAACAAGAAGTGAGTTAA | 200 |
| oligo fw | TAAAGGCAAAAACAGTGGTCGTGTGAGAAATC | |
| | TATTTTTTCGAAATTACTTACACTTTTGACGG | |
| | CTAGAAAAGGATATACATACATAT | |
| FMS1_repair | ATATGTATGTATATCCTTTTCTAGCCGTCAAA | 201 |
| oligo rv | AGTGTAAGTAATTTCGAAAAAATAGATTTCTC | |
| | ACACGACCACTGTTTTTGCCTTTATTAACTCA | |
| | CTTCTTGTTGGGAACAAAAAAAGA | |
| SPE3_target | TGCGCATGTTTCGGCGTTCGAAACTTCTCCGC | 202 |
| RNA FW | AGTGAAAGATAAATGATCATCTTCAAATCCAC | |
| | TACATAGTTTTAGAGCTAGAAATAGCAAGTTA | |
| | AAATAAGGCTAGTCCGTTATCAAC | |
| SPE3_target | GTTGATAACGGACTAGCCTTATTTTAACTTGC | 203 |
| RNA RV | TATTTCTAGCTCTAAAACTATGTAGTGGATTT | |
| | GAAGATGATCATTTATCTTTCACTGCGGAGAA | |
| | GTTTCGAACGCCGAAACATGCGCA | |
| SPE3_repair | TTGTACGCTTCACATAGTAGTTCAGTCAAGAA | 204 |
| oligo fw | GAGCAAACACTAATAAGCAATAAATCTAGGAG | |
| | AATATACATATATATGCATATGTTTGTTTAGC | |
| | TAAATAATTTTATTGAGCTTTGCT | |
| SPE3_repair | AGCAAAGCTCAATAAAATTATTTAGCTAAACA | 205 |
| oligo rv | AACATATGCATATATATGTATATTCTCCTAGA | |
| | TTTATTGCTTATTAGTGTTTGCTCTTCTTGAC | |
| | TGAACTACTATGTGAAGCGTACAA | |
| PR524_SPE3_ | ACACCAATATTCTGCACCTGC | 206 |
| dg fw | | |
| PR525_SPE3_ | TGCTGGAGAAGATCGTACGC | 207 |
| dg rv | | |
| SPE2_target | TGCGCATGTTTCGGCGTTCGAAACTTCTCCGC | 208 |
| RNA FW | AGTGAAAGATAAATGATCTTAGTAGTTTTTGG | |
| | AAGGATGTTTTAGAGCTAGAAATAGCAAGTTA | |
| | AAATAAGGCTAGTCCGTTATCAAC | |
| SPE2_target | GTTGATAACGGACTAGCCTTATTTTAACTTGC | 209 |
| RNA RV | TATTTCTAGCTCTAAAACATCCTTCCAAAAAC | |
| | TACTAAGATCATTTATCTTTCACTGCGGAGAA | |
| | GTTTCGAACGCCGAAACATGCGCA | |
| SPE2_repair | TTCTTTTTTATATTTTTTAGGTTTTCATATAG | 210 |
| oligo fw | TGTCTTACGCAAATAGGCGGACCATAGAAAAG | |
| | CCGCCATTTGTGTCTCCTCATACTTACATAGA | |
| | ATAGCCCTCTTCTATTATCCTTCG | |
| SPE2_repair | CGAAGGATAATAGAAGAGGGCTATTCTATGTA | 211 |
| oligo rv | AGTATGAGGAGACACAAATGGCGGCTTTTCTA | |
| | TGGTCCGCCTATTTGCGTAAGACACTATATGA | |
| | AAACCTAAAAAATATAAAAAAGAA | |
| SPE2_dg fw | TACAGCTCGCTCCTTGCATC | 212 |
| SPE2_dg rv | GCTTGCTTGGAGGGCTTTTC | 213 |
| SPE1_target | TGCGCATGTTTCGGCGTTCGAAACTTCTCCGC | 214 |
| RNA FW | AGTGAAAGATAAATGATCAAGAACCCTTTATC | |
| | ATAATTGTTTTAGAGCTAGAAATAGCAAGTTA | |
| | AAATAAGGCTAGTCCGTTATCAAC | |
| SPE1_target | GTTGATAACGGACTAGCCTTATTTTAACTTGC | 215 |
| RNA RV | TATTTCTAGCTCTAAAACAATTATGATAAAGG | |
| | GTTCTTGATCATTTATCTTTCACTGCGGAGAA | |
| | GTTTCGAACGCCGAAACATGCGCA | |
| SPE1_repair | TTTTTTGATTGTTCTACAACTTTTTCATAGTA | 216 |
| oligo fw | ATCAAAACCTTTGAATTTCAAACTTACTAGGA | |
| | TATATTTAACCACGACTTTCGCAAGAGAGACG | |
| | GAGGGGGTGGGAAAAGGCTGAATG | |
| SPE1_repair | CATTCAGCCTTTTCCCACCCCCTCCGTCTCTC | 217 |
| oligo rv | TTGCGAAAGTCGTGGTTAAATATATCCTAGTA | |
| | AGTTTGAAATTCAAAGGTTTTGATTACTATGA | |
| | AAAAGTTGTAGAACAATCAAAAAA | |
| SPE1_dg fw | TCATCCAGGTTTCAGCACGG | 218 |
| SPE1_dg rv | AGCTCGAACAAGGTGTCAGG | 219 |
| 550_(XI- | GATTACTTACCAATGTGCCATAAACTCCGTGC | 220 |
| 5_up)TEF1p- | ACCAATAGCTTCAAAATGTTTCTACTCC | |
| fw | | |
| 551_PRM9t | CTTGGGTTGTGGGCAATTGGGTGTACTATGAA | 221 |
| (XI-5_dw) | GCATTTTCAACATCGTATTTTCCGAAGC | |
| 552_(TEF1p) | GCAATCTAATCTAAGTTTTAATTACAAAATGG | 222 |
| SPE3-fw | CACAAGAAATCACTC | |
| 553_SPE3 | GTGCTAGTGTCTCCCGTCTTCTGTCTAATTTA | 223 |
| (PRM9t)-rv | ATTCCTTGGCTGC | |
| 554_XI-5- | GACCATCACTAAAGCTTCTCTCTTA | 224 |
| fw(dg) | | |
| 555_XI-5- | TTGAGCAATTCATCGACAACAAGAG | 225 |
| rv(dg) | | |
| 556_SPE2 | AGAACCAGAACCAGATCCTATTTTCTTCTGCA | 226 |
| (SPE3)-rv | ATTTCATATAG | |
| 557_(SPE2) | GGATCTGGTTCTGGTTCTATGGCACAAGAAAT | 227 |
| SPE3-fw | CACTC | |
| 558_SPE3 | AGAACCAGAACCAGATCCATTTAATTCCTTGG | 228 |
| (SPE2)-rv | CTGC | |
| 559_(SPE3) | GGATCTGGTTCTGGTTCTATGACTGTCACCAT | 229 |
| SPE2-fw | AAAAGAATTG | |
| 560_pYX21 | GATTACTTACCAATGTGCCATAAACTCCGTGC | 230 |
| 2t(XI-5)-rv | ACCATGCCGTAAACCACTAAATCGGAAC | |
| AAH1_target | TGCGCATGTTTCGGCGTTCGAAACTTCTCCGC | 231 |
| RNA FW | AGTGAAAGATAAATGATCTTCTTAGATTACTA | |
| | TTATATGTTTTAGAGCTAGAAATAGCAAGTTA | |
| | AAATAAGGCTAGTCCGTTATCAAC | |
| AAH1_target | GTTGATAACGGACTAGCCTTATTTTAACTTGC | 232 |
| RNA RV | TATTTCTAGCTCTAAAACATATAATAGTAATC | |
| | TAAGAAGATCATTTATCTTTCACTGCGGAGAA | |
| | GTTTCGAACGCCGAAACATGCGCA | |
| AAH1_repair | ATAGTTATTTTGAAATAATAACTACCATTAGA | 233 |
| oligo fw | ACTAACAAAAGAAAAGAAAAAAAAAATATACC | |
| | ATTTGCAAGACATTGTATAATATTTTTGTTGA | |
| | AAGTCTTTTTCGATTCATAAGCGC | |
| AAH1_repair | GCGCTTATGAATCGAAAAAGACTTTCAACAAA | 234 |
| oligo rv | AATATTATACAATGTCTTGCAAATGGTATATT | |
| | TTTTTTTTCTTTTCTTTTGTTAGTTCTAATGG | |
| | TAGTTATTATTTCAAAATAACTAT | |
| AAH1_dg fw | CTCATCGCATGCCAACGAAG | 235 |
| AAH1_dg rv | GCAGCAAAGCCAACCCTTAC | 236 |
| 579_(TEF1p) | GCAATCTAATCTAAGTTTTAATTACAAAATGT | 237 |
| LiMAT-fw | CTGTTCACTCTATCTTG | |
| 580_LIMAT | GTGCTAGTGTCTCCCGTCTTCTGTTTATTCAA | 238 |
| (PRM9t)-rv | CCATCTTCTTTGG | |
| 595_ARG3_ | CAATGTAGCGCTCTTACTTTATTATTTCAAGT | 239 |
| rv(DIT1t) | CCTTGAAATTACC | |
| 2602_GAPp_ | CTGGAGCTCAGTTTATCATTAT | 240 |
| Fw | | |
| 603_CYC1t_ | ACTATAGGGCGAATTGGGTAC | 241 |
| Rv | | |
| 604-X-3-up- | ATAATGATAAACTGAGCTCCAGAGTCTCGTAT | 242 |
| Rv | GTCGGCTC | |
| 605-X-3- | GTACCCAATTCGCCCTATAGTTGTGTCCGCGT | 243 |
| down-Fw | TTCTAAG | |
| 610_LIMAT | GGTAGACCAATGTAGCGCTCTTACTTTATTAT | 244 |
| rv(DIT1t) | TCAACCATCTTCTTTGGAAC | |
| 611_(TDH3p) | GTTTCGAATAAACACACATAAACAAACAAAAT | 245 |
| LiMAT_fw | GTCTGTTCACTCTATCTTGT | |
| 612_(TPI1p) | CTATAACTACAAAAAACACATACATAAACTAA | 246 |
| NCP1_fw | AAATGCCGTTTGGAATAGACAACAC | |
| 613_NCP1 | GACTAATAATTCTTAGTTAAAAGCACTTTACC | 247 |
| rv(ENO2t) | AGACATCTTCTTGGTATC | |
| 618_(dg)LiM | TGGTCACACAACTTGTCTG | 248 |
| AT_rv | | |
| 619_(dg)LiM | GGTACTGGTGGTTTCACTTG | 249 |
| AT_fw | | |
| 622_XII-5- | GTAGTGATCATTGGCTTAACG | 250 |
| up_fw | | |
| 623_XII-5- | GTTCCGATTTAGTGGTTTACGGCAGTGACAAT | 251 |
| up | AAATTCAAACCGGT | |
| (Pyx212t)_ | | |
| rv | | |
| 624_(PRM9t) | GCTTCGGAAAATACGATGTTGAAAATCAACTC | 252 |
| XII-5- | AGAAGTTTGACAGC | |
| dw_fw | | |
| 625_XII-5- | TCGTTAGATTCTGTATCCCTA | 253 |
| dw_rv | | |
| 626_XII-5- | GGAGTAGAAACATTTTGAAGCTATGTGACAAT | 254 |
| up(TEF1p)_ | AAATTCAAACCGGT | |
| rv | | |
| 635_ | ATTTGTGATCGAAGACGAAGAG | 255 |
| (dg)SPE2-rv | | |
| 636_ | TCAAGAAGCCACTACGTG | 256 |
| (dg)SPE3-fw | | |
| 637_ | ACTAGAACATTACCATATGTAGTG | 257 |
| (dg)SPE3-rv | | |
| 638_ | CAACTTGGACGTTCTTCTAC | 258 |
| (dg)SPE2-fw | | |

TABLE 2
plasmids

| | | Overexpressed | Yeast codon | Backbone |
|---|---|---|---|---|
| No. | Plasmids | gene/genes | optimized | plasmid |
| 1 | BvHSS_p426GPD | BvHSS | Yes | p426GPD |
| 2 | SvHSS_p426GPD | SvHSS | Yes | p426GPD |
| 3 | ScSPE4_p426GPD | ScSPE4 | Yes | p426GPD |
| 4 | AtACL5_p426GPD | AtACL5 | Yes | p426GPD |
| 5 | AtSPMS_p426GPD | AtSPMS | Yes | p426GPD |
| 6 | TbbTryS_p426GPD | TbbTryS | Yes | p426GPD |

TABLE 3
strains

| | | Backbone | | Genetic modifications in the |
|---|---|---|---|---|
| No. | Strain | strain | Plasmid | chromosome |
| 0 | JQSPD_AA | CEN.PK | | MATa SUC2 MAL2-8c ura3-52 |
| | | 113-11C | | his3-Δ1 ARG3p::KEX2p car2Δ, |
| | | | | AGC1, ORT1, GDH1, oaz1Δ, |
| | | | | SPE3, SPE1, SPE2, spe4Δ, |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |
| 1 | JQSPD_AA | JQSPD_AA | BvHSS_p426GPD | MATa SUC2 MAL2-8c ura3-52 |
| | (BvHSS_p426GPD) | | | his3-Δ1 ARG3p::KEX2p car2Δ, |
| | | | | AGC1, ORT1, GDH1, oaz1Δ, |
| | | | | SPE3, SPE1, SPE2, spe4Δ, |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |
| 2 | JQSPD_AA | JQSPD_AA | SvHSS_p426GPD | MATa SUC2 MAL2-8c ura3-52 |
| | (SvHSS_p426GPD) | | | his3-Δ1 ARG3p::KEX2p car2Δ, |
| | | | | AGC1, ORT1, GDH1, oaz1Δ, |
| | | | | SPE3, SPE1, SPE2, spe4Δ, |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |
| 3 | JQSPD_AA | JQSPD_AA | ScSPE4_p426GPD | MATa SUC2 MAL2-8c ura3-52 |
| | (ScSPE4_p426GPD) | | | his3-Δ1 ARG3p::KEX2p car2Δ, |
| | | | | AGC1, ORT1, GDH1, oaz1Δ, |
| | | | | SPE3, SPE1, SPE2, spe4Δ, |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |
| 4 | JQSPD_AA | JQSPD_AA | AtSPMS_p426GPD | MATa SUC2 MAL2-8c ura3-52 |
| | (AtSPMS_p426GPD) | | | his3-Δ1 ARG3p::KEX2p car2Δ, |
| | | | | AGC1, ORT1, GDH1, oaz1Δ, |
| | | | | SPE3, SPE1, SPE2, spe4Δ, |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |
| 5 | JQSPD_AA | JQSPD_AA | AtACL5_p426GPD | MATa SUC2 MAL2-8c ura3-52 |
| | (AtACL5_p426GPD) | | | his3-Δ1 ARG3p::KEX2p car2Δ, |
| | | | | AGC1, ORT1, GDH1, oaz1Δ, |
| | | | | SPE3, SPE1, SPE2, spe4Δ, |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |
| 6 | JQSPD_AA | JQSPD_AA | TbbTryS_p426GPD | MATa SUC2 MAL2-8c ura3-52 |
| | (TbbTryS_p426GPD) | | | his3-Δ1 ARG3p::KEX2p car2Δ, |
| | | | | AGC1, ORT1, GDH1, oaz1Δ, |
| | | | | SPE3, SPE1, SPE2, spe4Δ, |
| | | | | CgargJ, EcargB, CgargC, |
| | | | | CgargD, EcargA, MEU1p, BAT2, |
| | | | | APT1, PRS5, Δfms1, Δaah1, |
| | | | | LiMAT, TPO5, SPE2-SPE3 |

An assay combining deep-well scale fermentation with high-performance liquid chromatography (HPLC) used to assess the resulting strain JQSPD_AA. In particular, 24 deep-well batch fermentations of the resulting JQSPD_AA strain for production of polyamines carried out in minimal medium developed by Verduyn et al 1992. Cultures were inoculated from 24 h precultures at an initial OD₆₀₀ of 0.2 with 2 ml minimal medium in 24 deep-well plates and cultivated at 300 rpm, 30° C. for 120 hours. The minimal medium containing 7.5 g/l (NH₄)₂SO₄, 14.4 g/l KH₂PO₄, 0.5 g/l MgSO₄·7H₂O, 20 g/l glucose, 2 ml/l trace metal and 1 ml/l vitamin solutions supplemented with 40 mg/l uracil, 40 mg/l histidine if needed, pH was adjust to 4.5. Sample was prepared by taking 0.1 ml of liquid culture and was subject to hot water (HW) extraction. In the method, we used minimal medium in the deep-well plate's fermentation as the extract context. Tubes containing 0.9 ml of fermentation medium were preheated in a water bath at 100° C. for 10 min. Then, the hot fermentation medium was quickly poured over the 0.1 ml of liquid culture; the mixture was immediately vortexed, and the sample was placed in the water bath. After 30 min, each tube was placed on ice for 5 min. After centrifugation, the supernatant was directly used for derivatization. For derivatization, 0.125 ml of saturated NaHCO₃ solution and 0.25 ml of dansyl chloride solution (5 mg/ml in acetone) were added to 0.25 ml of sample. Then the reaction mixture was incubated at 40° C. for 1 h in the dark with occasional shaking. The reaction was stopped by adding 0.275 ml of methanol. Samples were filtered through a 25 mm syringe filter (0.45 μm Nylon) used for HPLC detection. The following chromatographic condition were used: C18 (100 mm×4.6 mm i.d., 2.6 μm, Phenomenex Kinetex), excitation wavelength 340 nm, emission wavelength 515 nm, sample injecting 1.5 μl, column temperature 40° C., Detector sensitivity 7, acquisition starts at 4.0 min. The mobile phase was water and methanol with the speed of 1 ml/min. The elution program was as follows: 0-5 min 50% to 65% methanol, 5-7.5 min 65% to 75% methanol, 7.5-9.5 min 75% to 87.5% methanol, 9.5-10.5 min 87.5% to 100% methanol, 10.5-11.5 min 100% methanol, 11.5-13.5 min 100% to 50% methanol, 13.5-16 50% methanol.

Strain JQSPD_AA produced Spd titer at the concentration >400 mg/l, significantly increased Spd titer compared to the strains with only partial of the modifications as used herein (see the examples in WO 2016/144247 and WO 2019/013696).

### Example 2: Production of Higher Polyamines in Yeast

Life has evolved diversiform pathways to synthesize structural variants of polyamines. Indeed, while Put and Spd are typically found in most cells as common polyamines, uncommon polyamines, such as sym-homospermidine (Hspd), thermospermine (Tspm), spermine (Spm), branched chain polyamines, and long-chain polyamines (LCPAs) have also been identified in nature. This Example 2 investigated biosynthesis of sym-homospermidine (Hspd), thermospermine (Tspm) and spermine (Spm) by designing and introducing a genetic module (VII) into the Spd platform strain JQSPD_AA from Example 1.

We first set out to heterologously synthesize triamine Hspd, which exist in both plants and bacterial. In plants, Hspd is the first pathway specific intermediate in pyrrolizidine alkaloids biosynthesis, which is formed by homospermidine synthase (plant HSS; EC 2.5.1.45). This enzyme is more specific than bacteria homospermidine synthase (bacteria HSS; EC 2.5.1.44), as the latter cannot use Put as donor of the aminobutyl group. To explore the possibility of both plant and bacteria HSS for Hspd microbial production, genetic sub-modules (VII-a) and (VII-b) designed to biosynthesis of Hspd in yeast encoded the expression of Senecio vernalis SvHSS and Blastochloris viridis BvHSS13 respectively. The sub-modules were introduced as high-copy plasmid SvHSS_p426GPD and BvHSS_p426GPD wre ordered from GenScipt and harbored the yeast codon-optimized SvHSS gene [SEQ ID NO: 8] and BvHSS gene [SEQ ID NO: 10] respectively into the Spd platform strain JQSPD_AA. The transformation experiment followed the same procedure as in Example 1. The resulting strain JQSPD_AA (SvHSS_p426GPD) and JQSPD_AA (BvHSS_p426GPD) were assayed for Hspd production with the same procedure as descripted in Example 1.

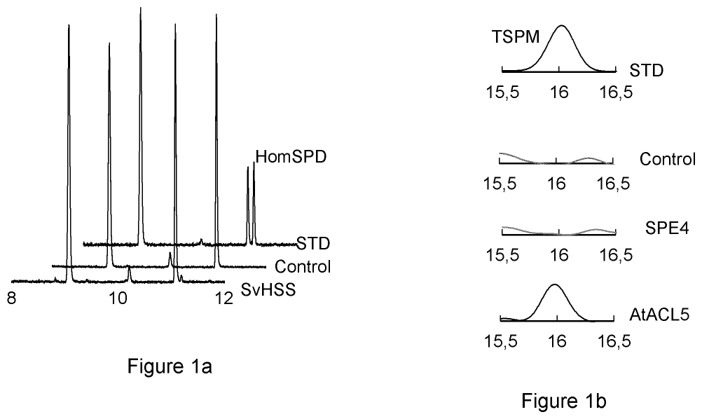

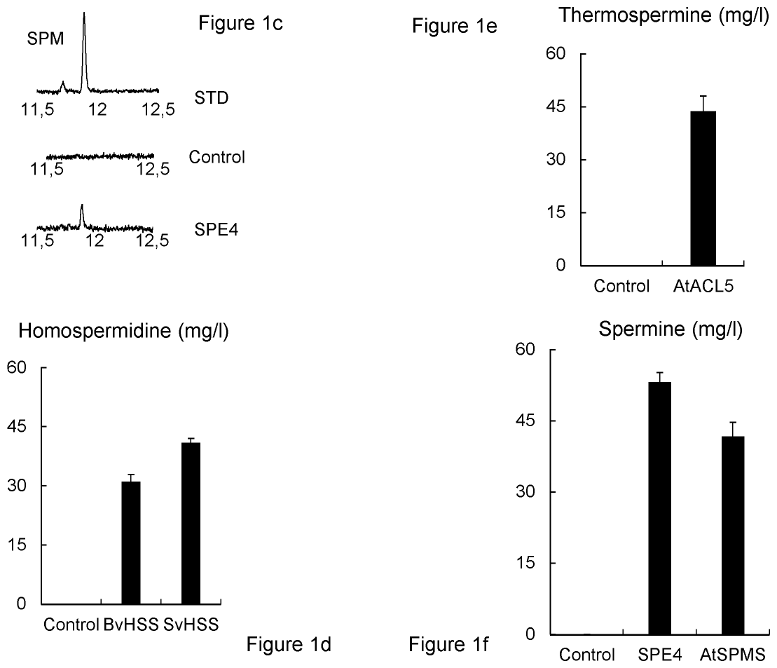

We found that the overexpression of both HSS enabled the biosynthesis of Hspd. In particular, SvHSS enabled Hspd titer at 40.9 mg/, while BvHSS enabled Hspd titer at 31.1 mg/(see FIGS. 1a and 1d).

Subsequently, we also exploited the Spd platform (Example 1) for the production of tetra-amines Spm and Tspm by introducing sub-modules (VII-c), sub-modules (VII-d) and sub-modules (VII-e). Spm is the most common tetra-amine that is found throughout the metazoa, in flowering plants and in yeast. A specific aminopropyltransferase, i.e., spermine synthase (SpmSyn; EC 2.5.1.22) is responsible for Spm biosynthesis. We first explored the yeast native SpmSyn Spe4p for Spm over-production.

When over-expressed codon-optimized yeast SPE4 [SEQ ID NO: 2] as a high-copy plasmid SPE4_p426GPD (sub-module (VII-c) in JQSPD_AA, 53.1 mg/l of Spm was obtained (see FIGS. 1c and 1f). We also tested the SpmSyn from Arabidopsis thaliana by over-expressed AtSPMS [SEQ ID NO: 4] as a high-copy plasmid AtSPMS_p426GPD (sub-module (VII-d)) in the JQSPD_AA strain. This resulted in production of Spm (41.8 mg/l; see FIGS. 1c and 1f). The plant ACL5 aminopropyltransferase (TspmSyn; EC 2.5.1.79) from A. thaliana was shown to synthesize the Spm isomer Tspm. We thereby also over-expressed AtACL5 [SEQ ID NO: 6] as a high-copy plasmid AtACL5_p426GPD (sub-module (VII-e)) in the JQSPD_AA strain. This strategy enabled 43.8 mg/l of Tspm production (see FIGS. 1b and 1e). All the plasmids harboring the yeast codon-optimized genes were purchased from GenScript. The same transformation and product assay as used in the Example 1 was used in this Example 2. All plasmids are listed in Table 2 and all strains are listed in Table 3.

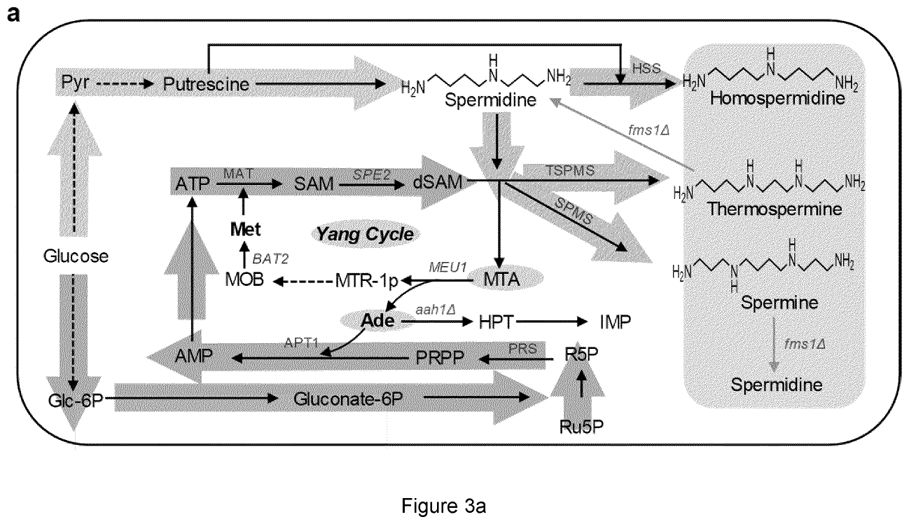

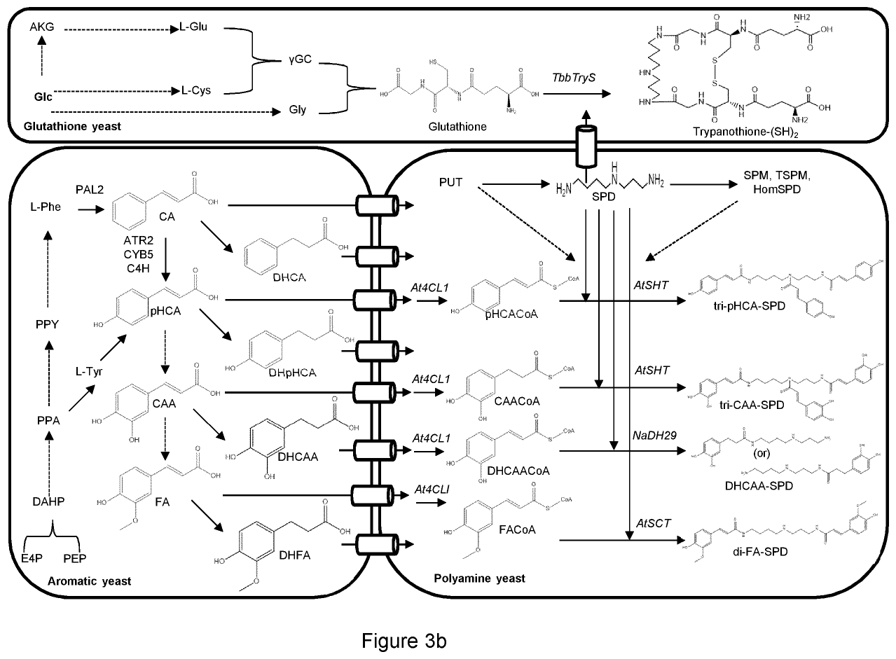

FIG. 3a illustrates engineered pathways for the biosynthesis of spermidine and higher polyamines in yeast.

### Example 3: Biosynthesis of Trypanothione in Yeast

The amide bond is undoubtedly one of the most important structural motifs in nature. Approximately a quarter of all marketed drugs and two-thirds of all drug candidates bear at least one amide bond, and the acylation of amine is one of the most widely practiced reactions in the pharmaceutical industry. Polyamines present unique scaffolds to attach other moieties to, and are often incorporated into specialized metabolism, leading to the biosynthesis of diverse amide bond containing nature products with complex structure. For instance, trypanothione (N¹,N¹⁰-bis(glutathionyl) spermidine, T(SH)₂), is the main low molecular weight thiol in trypanosomatids of the genera Crithidia, Trypanosoma, and Leishmania, the latter two comprising causative agents of life-threatening or disabling diseases, such as African sleeping sickness, kala azar, Chagas' disease, and espundia or oriental sore. Meanwhile, abundant polyamine-containing hydroxycinnamic amides, which have been postulated to be involved in flower, pollen and seed development and pathogen resistance, can be synthesized in plants. However, it is hard to obtain them from either traditional synthetic chemistry or extraction from the nature sources. Firstly, these polyamine conjugates exhibit low abundance in nature (Li et al. 2018). On the other hand, establishing an efficient catalytic system for direct amidation reactions has remained a formidable challenge for years in organic chemistry (Wang 2019). In this Example 3, we exploited the polyamine platform from Example 1 in the biosynthesis of polyamine conjugates by introducing module (VIII).

We first set out to heterologous biosynthesis of [T(SH)₂] in yeast. [T(SH)₂], the major redox mediator in pathogenic trypanosomatids, is synthetized stepwise by two distinct enzymes, i.e., glutathionylspermidine synthase (GspS; EC 6.3.1.8) and trypanothione synthase (TryS; EC 6.3.1.9) in Crithidia fasciculate from glutathione (GSH) and spermidine (Spd), whereas in Trypanosoma brucei brucei both steps are catalyzed by an unusual TryS (EC 6.3.1.9) with broad substrate specificity. To explore TbbTryS for [T(SH)₂] microbial production, genetic sub-modules (VIII-a) designed to synthesize [T(SH)₂] in yeast encoded the expression of T. brucei brucei TryS (TbbTryS). The sub-modules were introduced as high-copy plasmid TbbTryS_p426GPD that was ordered from GenScript harboring the yeast codon-optimized TbbTryS gene [SEQ ID NO: 35] to the Spd platform strain JQSPD_AA. The transformation experiment was conducted following the same procedure as descripted in Example 1. The resulting strain JQSPD_AA (TbbTryS_p426GPD) was used for a 24 deep-well based fermentation with the same procedure as descripted in Example 1. Fermentation sample was prepared by taking 0.1 ml of liquid culture. Fermentation sample was subject to hot water (HW) extraction, in our method we used fermentation medium to extract. Tubes containing 0.9 ml of fermentation medium were preheated in a water bath at 100° C. for 10 min. Then, the hot fermentation medium was quickly poured over the 0.1 ml of liquid culture; the mixture was immediately vortexed, and the sample was placed in the water bath. After 30 min, each tube was placed on ice for 5 min. After centrifugation, the supernatant was directly used for detection.

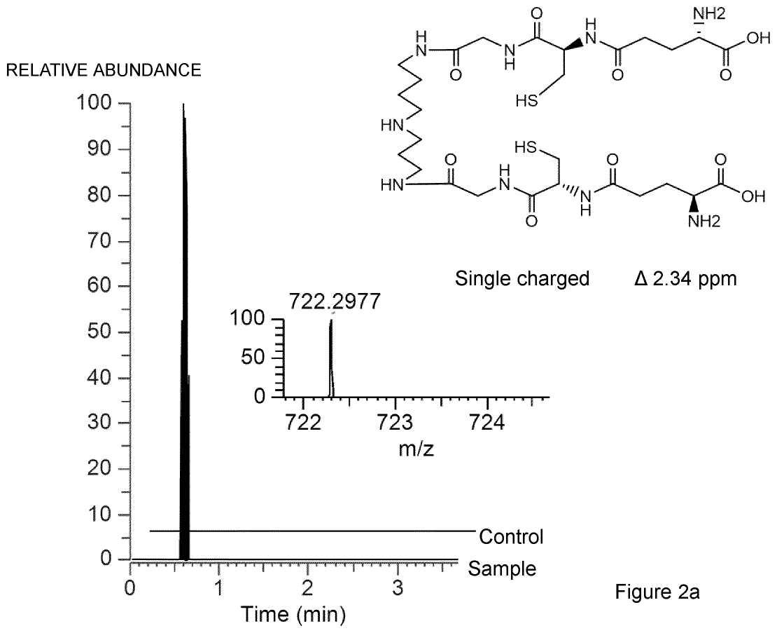

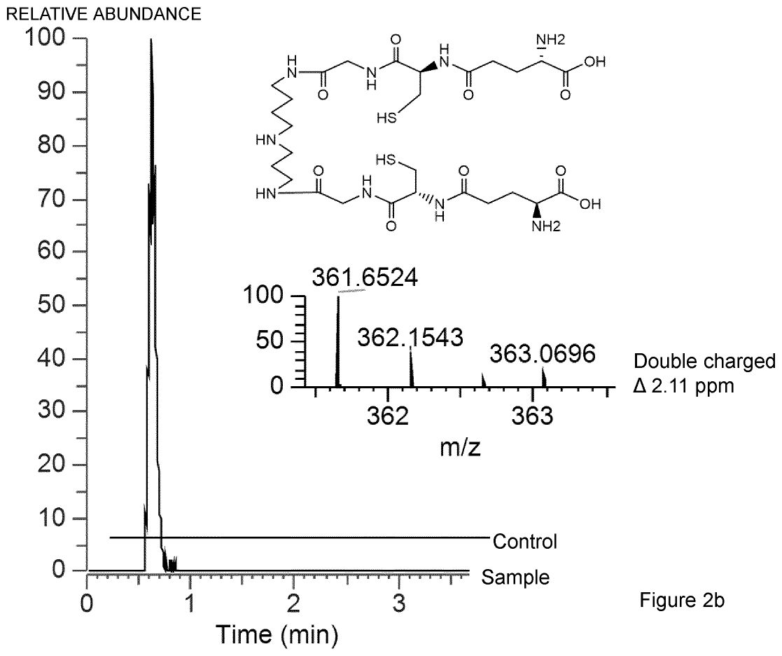

Detection of polyamine conjugates was conducted by liquid chromatography-mass spectrometry (LC-MS) measurements on a Dionex UltiMate 3000 UHPLC (Fisher Scientific, San Jose, CA) connected to an Orbitrap Fusion Mass Spectrometer (Thermo Fisher Scientific, San Jose, CA). The system used an Agilent Zorbax Eclipse Plus C18 2.1×100 mm, 1.8 μm column kept at 35° C. The flow rate was 0.350 mL/min with 0.1% formic acid (A) and 0.1% formic acid in acetonitrile (B) as mobile phase. The gradient started as 5% B for 1 min and then followed a linear gradient to 95% B until 5 min. This solvent composition was held for 1.5 min after which it was changed to 5% B and held until 8 min. The sample (5 μl) was passed on to the MS equipped with a heated electrospray ionization source (HESI) in positive-ion or positive-ion mode with sheath gas set to 50 (a.u.), aux gas to 10 (a.u.) and sweep gas to 1 (a.u.). The cone and probe temperature were 325° C. and 380° C., respectively, and spray voltage was 3500 V. Scan range was 80 to 500 Da and time between scans was 50 ms. Consistent with the property of TbbTrs to catalyze two GSH moieties onto spermidine, over-expression of this enzyme in the Spd platform JQSPD_AA from Example 1 resulted in [T(SH)₂] production. In particular, a single new LC-MS peak with a m/z value corresponding to 722.2977 [M+H]⁺ or 361.6524 [M+H]²⁺ was detected, indicating the presence of [T(SH)₂] (see FIGS. 2a and 2b). All plasmids are listed in Table 2 and all strains are listed in Table 3.

FIG. 3b illustrates engineered pathways for the biosynthesis of glutathione-polyamine conjugates in yeast.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

### REFERENCES

Bienz, S., Detterbeck R., Ensch C., Guggisberg A., Häusermann U., Meisterhans C., Wendt B., Werner C. & Hesse M. Putrescine, spermidine, spermine, and related polyamine alkaloids. Alkaloids Chem Biol. 58, 83-338 (2002).
Kim, S. K., Jin, Y. S., Choi, I. G., Park, Y. C. & Seo, J. H. Enhanced tolerance of Saccharomyces cerevisiae to multiple lignocellulose-derived inhibitors through modulation of spermidine contents. Metabolic Engineering 29, 46-55, doi:http://dx.doi.org/10.1016/j.ymben.2015.02.004 (2015).
Li, S., Li, Y. & Smolke, C. D. Strategies for microbial synthesis of high-value phytochemicals. Nat Chem 10, 395-404, doi:10.1038/s41557-018-0013-z (2018).
Mans, R., van Rossum, H. M., Wijsman, M., Backx, A., Kuijpers, N. G., van den Broek, M., Daran-Lapujade, P., Pronk, J. T., van Maris, A. J. & Daran, J. M. CRISPR/Cas9: a molecular Swiss army knife for simultaneous introduction of multiple genetic modifications in Saccharomyces cerevisiae. FEMS Yeast Res. 15, fov004-fov004, doi: 10.1093/femsyr/fov004 (2015).
Michael, A. J. Polyamines in Eukaryotes, Bacteria, and Archaea. J. Biol. Chem. 291, 14896-14903, doi:10.1074/jbc.R116.734780 (2016).
Nielsen, J. Yeast cell factories on the horizon. Science 349, 1050-1051, doi: 10.1126/science.aad2081 (2015).
Nielsen, J. Cell factory engineering for improved production of natural products. Natural Product Reports, doi:10.1039/C9NP00005D (2019).
Nielsen, J. & Keasling, Jay D. Engineering Cellular Metabolism. Cell 164, 1185-1197, doi:https://doi.org/10.1016/j.cell.2016.02.004 (2016).
Ober, D. & Hartmann, T. Homospermidine synthase, the first pathway-specific enzyme of pyrrolizidine alkaloid biosynthesis, evolved from deoxyhypusine synthase. Proceedings of the National Academy of Sciences 96, 14777-14782, doi:10.1073/pnas.96.26.14777 (1999).
Verduyn, C., Postma, E., Scheffers, W. A., Van Dijken, J. P. Effect of benzoic acid on metabolic fluxes in yeasts: A continuous-culture study on the regulation of respiration and alcoholic fermentation. Yeast 8, 501-517, doi:10.1002/yea.320080703 (1992).
Wang, X. Challenges and outlook for catalytic direct amidation reactions. Nature Catalysis 2, 98-102, doi:10.1038/s41929-018-0215-1 (2019).
Yu, T. Zhou, Y. J., Huang, M., Liu, Q., Pereira, R., David, F., & Nielsen, J. Reprogramming Yeast Metabolism from Alcoholic Fermentation to Lipogenesis. Cell 174, 1549-1558. e1514, doi:https://doi.org/10.1016/j.cell.2018.07.013 (2018).
Zhou, Y. J., Buijs, N. A., Zhu, Z., Qin, J., Siewers, V. & Nielsen, J. Production of fatty acid-derived oleochemicals and biofuels by synthetic yeast cell factories. Nat. Commun 7, 11709, doi:10.1038/ncomms11709, https://www.nature.com/articles/ncomms11709 #supplementary-information (2016).

1. A yeast cell capable of producing glutathione-polyamine conjugates, wherein
the yeast cell is capable of producing at least one polyamine;
the yeast cell comprises a polyamine:glutathione ligase encoding gene;
the yeast cell comprises at least one polyamine synthase encoding gene; and
the yeast cell lacks a polyamine oxidase encoding gene or comprises a disrupted polyamine oxidase encoding gene.

2. The yeast cell according to claim 1, wherein the yeast cell is engineered for overexpression of the polyamine: glutathione ligase.

3. The yeast cell according to claim 1 or 2, wherein the glutathione-polyamine conjugates are selected from the group consisting of trypanothione, N¹-glutathionyl spermidine, N¹⁰-glutathionyl spermidine, N¹,N¹⁰-bis(glutathionyl) spermine, N¹, N⁵, N¹⁰-tri(glutathionyl) spermine, and N¹,N⁵,N¹⁰,N¹⁴-tetra(glutathionyl) spermine.

4. The yeast cell according to any of the claims 1 to 3, wherein the polyamine: glutathione ligase encoding gene is selected from the group consisting of trypanothione synthase, glutathionylspermidine synthase, and a combination thereof.

5. The yeast cell according to claim 4, wherein the trypanothione synthase encoding gene is selected from the group consisting of Trypanosoma brucei brucei TbbTryS and a nucleotide sequence encoding a trypanothione synthase having at least 80% sequence identity with trypanothione synthase TbbTryS.

6. The yeast cell according to claim 4 or 5, wherein the glutathionylspermidine synthase (GSS) encoding gene is selected from the group consisting of Escherichia coli EcGSS and a nucleotide sequence encoding a glutathionylspermidine synthase having at least 80% sequence identity with glutathionylspermidine synthase EcGSS.

7. The yeast cell according to any of the claims 1 to 6, wherein the at least one polyamine is selected from the group consisting of spermine, thermospermine, sym-homospermidine, 1,3-diaminopropane, putrescine, cadaverine, agmatine, spermidine, sym-norspermidine, norspermine and a combination thereof.

8. The yeast cell according to any of the claims 1 to 7, wherein the yeast cell is engineered for overexpression of the at least one polyamine synthase.

9. The yeast cell according to any of the claims 1 to 8, wherein the polyamine synthase encoding gene is selected from the group consisting of a spermine synthase encoding gene, a thermospermine synthase encoding gene and a homospermidine synthase encoding gene.

10. The yeast cell according to claim 9, wherein the spermine synthase encoding gene is selected from the group consisting of Saccharomyces cerevisiae SPE4, Arabidopsis thaliana AtSPMS and a nucleotide sequence encoding a spermine synthase having at least 80% sequence identity with spermine synthase SPE4 or spermine synthase AtSPMS.

11. The yeast cell according to claim 9 or 10, wherein the thermospermine synthase encoding gene is selected from the group consisting of Arabidopsis thaliana AtACL5 and a nucleotide sequence encoding a thermospermine synthase having at least 80% sequence identity with thermospermine synthase AtACL5.

12. The yeast cell according to any of the claims 9 to 11, wherein the homospermidine synthase encoding gene is selected from the group consisting of Senecio vernalis SvHSS, Blastochloris viridis BvHSS and a nucleotide sequence encoding a homospermidine synthase having at least 80% sequence identity with homospermidine synthase SvHSS or homospermidine synthase BvHSS.

13. The yeast cell according to any of the claims 1 to 12, wherein the yeast cell is a Saccharomyces cerevisiae cell and the polyamine oxidase is FMS1.

14. A method of producing glutathione-polyamine conjugates, the method comprising:
culturing a yeast cell according to any of the claims 1 to 13 in a culture medium and in culture conditions suitable for production of the glutathione-polyamine conjugates by the yeast cell; and
collecting the glutathione-polyamine conjugates from the culture medium and/or from the yeast cell.
